(19) United States
(12) Patent Application Publication
(10) Pub. No.: US 2025/0102514 A1
(43) Pub. Date: Mar. 27, 2025

(54) PARTICLES AND METHODS OF ASSAYING

(71) Applicant: Seer, Inc., Redwood City, CA (US)

(72) Inventors: Daniel Hornburg, Foster City, CA (US); Shadi Roshdiferdosi, San Carlos, CA (US); Moaraj Hasan, San Francisco, CA (US); Eddie Eugene Wang, Belmont, CA (US); David Lee Chinn, Orinda, CA (US)

(21) Appl. No.: 18/730,551

(22) PCT Filed: Jan. 20, 2023
(86) PCT No.: PCT/US2023/061012

§ 371 Date: Jul. 19, 2024

Related U.S. Application Data
(60) Provisional application No. 63/301,489, filed on Jan. 20, 2022.

(51) Int. Cl. G01N 33/68; G01N 33/543

(52) U.S. Cl. CPC G01N 33/6848; G01N 33/54346; G01N 2458/10

(57) ABSTRACT

The present disclosure describes a method for determining a concentration or an amount of a biomolecule or biomolecule group in a biological sample, the method comprising: (a) contacting said biological sample with a plurality of particle-containing solutions each having a different particle concentration, to generate a plurality of biomolecule coronas each corresponding to an individual solution of said plurality of particle-containing solutions: (b) assaying said plurality of biomolecule coronas for a dataset comprising data corresponding to one or more biomolecules or biomolecule groups comprising said biomolecule or biomolecule group in said biological sample; and determining said concentration or said amount of said biomolecule or said biomolecule group in said biological sample based at least partially on said dataset, wherein said determining is made in the absence of using a reference biomolecule external to said biological sample.

### CROSS-REFERENCE

This application claims benefit of U.S. Provisional Application No. 63/301,489, filed on Jan. 20, 2022, which is incorporated herein by reference in its entirety.

### BACKGROUND

Biological samples such as biofluids contain a wide variety of proteins whose presence, processing, and relative abundances may be indicative of biological state. High abundance proteins and other proteins may overshadow the signal relative to other proteins in an assay. Sample preparation, such as dilution, can further overshadow the relative signals in an assay.

### SUMMARY

Many biomolecule collection systems, for example many biomolecule corona-generating substrates, are inherently limited by off-target analyte binding and target molecule dynamic exchange, and therefore provide limited sensitivities and profiling depths. Recognized herein is a need for repeatable and quantitative analytical methods for low abundance biomolecule identification. The present disclosure provides a range of systems, compositions, and strategies for expanding dynamic range and profiling depth for targeted biomolecule collection and analysis. In certain aspects, the present disclosure provides methods for tailoring substrate mass and surface area ratios for targeted biomolecule collection. The present disclosure further provides strategies for generating quantitative trends in biomolecular data, enabling direct deep compositional analysis of biological samples with minimal sample perturbation.

In some aspects, the present disclosure describes a method for determining a concentration or an amount of a biomolecule or biomolecule group in a biological sample, the method comprising: (a) contacting said biological sample with a plurality of particle-containing solutions each having a different particle concentration, to generate a plurality of biomolecule coronas each corresponding to an individual solution of said plurality of particle-containing solutions; (b) assaying said plurality of biomolecule coronas for a dataset comprising data corresponding to one or more biomolecules or biomolecule groups comprising said biomolecule or biomolecule group in said biological sample; and (c) determining said concentration or said amount of said biomolecule or said biomolecule group in said biological sample based at least partially on said dataset, wherein said determining is made in the absence of using a reference biomolecule external to said biological sample.

In some embodiments, at least a subset of said plurality of particle-containing solutions differ in particle concentration by at least 1 order of magnitude.

In some embodiments, solutions of said plurality of particle-containing solutions have particle concentrations between 1 μg/ml and 100 mg/ml.

In some embodiments, solutions of said plurality of particle-containing solutions have particle concentrations of at least 50 μg/ml.

In some embodiments, said plurality of biomolecule coronas are associated with a single particle type in solutions of said plurality of particle-containing solutions.

In some embodiments, each of said particle-containing solutions comprises a same particle type.

In some embodiments, each of said particle-containing solutions comprises a same particle panel comprising a plurality of different particles.

In some embodiments, particles in an individual solution of said plurality of particle-containing solutions have a polydispersity of less than 1.

In some embodiments, particles in an individual solution of said plurality of particle-containing solutions have a polydispersity of less than 0.5.

In some embodiments, said polydispersity is determined at least in part by size variance of said particles.

In some embodiments, said polydispersity is determined at least in part by mass variance of said particles.

In some embodiments, a solution of said plurality of particle-containing solutions comprises a surface modified particle.

In some embodiments, a solution of said plurality of particle-containing solutions comprises a plurality of surface modified particles.

In some embodiments, said plurality of surface modified particles comprises particles having different physicochemical properties.

In some embodiments, said physicochemical properties comprise size, charge, core material, shell material, porosity, density, hydrophobicity, hydrophilicity, charge, rigidity, or any combination thereof.

In some embodiments, said dataset comprises a plurality of signals corresponding to said plurality of biomolecule coronas.

In some embodiments, said dataset comprises a plurality of datasets.

In some embodiments, said plurality of signals comprises optical signals, electrical signals, or a combination thereof.

In some embodiments, said determining of (c) comprises comparing intensities of said plurality of signals against an intensity of a reference signal.

In some embodiments, said reference signal is associated with a biomolecule intrinsic to said sample.

In some embodiments, said biomolecule intrinsic to said sample comprises albumin, globulin, transferrin, fibrinogen, antitrypsin, al-acid glycoprotein, apolipoprotein, ceruloplasmin, transthyretin, a complement factor, or any combination thereof.

In some embodiments, said dataset comprises training data for a machine learning algorithm.

In some embodiments, said contacting of (c) for each of said plurality of particle-containing solutions is for identical lengths of time.

In some embodiments, said identical lengths of time are shorter than the equilibration times of said plurality of particle-containing solutions subsequent to said contacting of (a).

In some embodiments, said one or more biomolecules or biomolecule groups comprise a plurality of biomolecules or biomolecule groups, and wherein said determining of (c) comprises identifying a concentration or an amount of each of said plurality of biomolecules or biomolecule groups in said biological sample.

In some embodiments, concentrations of said plurality of biomolecules or biomolecule groups are identified in a single assay.

In some embodiments, said biomolecule or biomolecule group comprises a protein or protein group.

In some embodiments, said concentration of said biomolecule or said biomolecule group is less than about 10 μg/ml.

In some embodiments, said concentration of said biomolecule or said biomolecule group is less than about 1 μg/ml.

In some embodiments, said concentration of said biomolecule or said biomolecule group is less than about 100 ng/ml.

In some embodiments, said concentration of said biomolecule or said biomolecule group is less than about 10 ng/ml.

In some embodiments, said concentration of said biomolecule or said biomolecule group is less than about 1 ng/ml.

In some embodiments, said concentration of said biomolecule or said biomolecule group is less than about 100 μg/ml.

In some embodiments, said biomolecule or biomolecule group comprises a plurality of human plasma proteins or human plasma protein groups, and wherein said plurality of human plasma proteins or human plasma protein groups comprises at least 20 proteins or protein groups.

In some embodiments, said determining of (c) comprises determining concentrations of said at least 20 proteins or protein groups based at least partially on intensities of said plurality of signals.

In some embodiments, said assaying of (b) comprises separating said plurality of biomolecule coronas from said biological sample.

In some embodiments, said separating comprises magnetically separating said plurality of biomolecule coronas from said biological sample.

In some embodiments, said assaying of (b) comprises digesting said one or more biomolecules or biomolecule groups.

In some embodiments, said determining of (c) comprises identifying relative abundances of a plurality of isoforms of a protein.

In some embodiments, a particle concentration of said plurality of said particle-containing solutions is approximately equal to a total protein concentration of said biological sample.

In some embodiments, said contacting said biological sample with said plurality of particle-containing solutions comprises combining at most about 250 μL of said biological sample with at most about 250 μL of a particle-containing solution of said plurality of particle-containing solutions.

In some embodiments, said contacting said biological sample with said plurality of particle-containing solutions comprises combining at most about 100 μL of said biological sample with at most about 100 μL of a particle-containing solution of said plurality of particle-containing solutions.

In some embodiments, said contacting said biological sample with said plurality of particle-containing solutions comprises adding at least about 100 nL of plasma per cm² of particle surface area to each solution of said plurality of particle-containing solutions.

In some embodiments, said contacting said biological sample with said plurality of particle-containing solutions comprises adding between about 100 nL and 100 mL of plasma per cm² of particle surface area to each solution of said plurality of particle-containing solutions.

In some embodiments, said biological sample is diluted by at least 2-fold prior to said contacting with said plurality of particle-containing solutions.

In some embodiments, said biological sample is diluted by at least 5-fold prior to said contacting with said plurality of particle-containing solutions.

In some embodiments, particles of said plurality of particle-containing solutions have diameters between about 100 and about 500 nanometers.

In some embodiments, particles of said plurality of particle-containing solutions have diameters between about 100 and about 300 nanometers.

In some embodiments, particles of said plurality of particle-containing solutions comprise diameters of at least about 500 nanometers.

In some embodiments, particles of said plurality of particle-containing solutions comprise diameters of at most about 200 nanometers.

In some embodiments, said plurality of particle-containing solutions comprises a particle selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating.

In some embodiments, said plurality of particle-containing solutions comprises at least two particles selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating.

In some embodiments, said plurality of particle-containing solutions comprises at least three particles selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating.

In some embodiments, said plurality of particle-containing solutions comprises at least four particles selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating.

In some embodiments, said plurality of particle-containing solutions comprises a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating.

In some embodiments, said plurality of particle-containing solutions comprises a superparamagnetic iron oxide particle (SPION) comprising a poly(N-(3-(dimethylamino) propyl) methacrylamide) (PDMAPMA) surface.

In some embodiments, said plurality of particle-containing solutions comprises a superparamagnetic iron oxide particle (SPION) comprising a poly(oligo (ethylene glycol) methyl ether methacrylate) (POEGMA) surface.

In some embodiments, said plurality of particle-containing solutions comprises a superparamagnetic iron oxide particle (SPION) comprising an N-(3-Trimethoxysilylpropyl) diethylenetriamine surface.

In some embodiments, said plurality of particle-containing solutions comprises a superparamagnetic iron oxide particle (SPION) comprising a Poly(dimethyl aminopropyl methacrylamide) (Dimethylamine) surface.

In some embodiments, said plurality of particle-containing solutions comprises a superparamagnetic iron oxide particle (SPION) comprising a dextran surface.

In some embodiments, said plurality of particle-containing solutions comprises a superparamagnetic iron oxide particle (SPION) comprising a surface with a mixed chemistry based on amine-epoxy chemistry.

In some embodiments, said plurality of particle-containing solutions comprises a superparamagnetic iron oxide particle (SPION) comprising a Polyzwitterion coated (Poly(N-[3-(Dimethylamino) propyl]methacrylamide-co-[2-(methacryloyloxy)ethyl]dimethyl-(3-sulfopropyl) ammonium hydroxide, P (DMAPMA-co-SBMA)) surface.

In some embodiments, said plurality of particle-containing solutions comprises a superparamagnetic iron oxide particle (SPION) comprising styrene surface comprising an oleic acid functionalization.

In some embodiments, said plurality of particle-containing solutions comprises a superparamagnetic iron oxide particle (SPION) comprising a boronated styrene surface.

In some embodiments, said plurality of particle-containing solutions comprises a superparamagnetic iron oxide particle (SPION) comprising a carboxylated styrene surface.

In some embodiments, said plurality of particle-containing solutions comprises a superparamagnetic iron oxide microparticle (SPION) comprising a carboxylated styrene surface.

In some embodiments, said plurality of particle-containing solutions comprises a superparamagnetic iron oxide microparticle (SPION) comprising a strongly acidic silica surface.

In some aspects, the present disclosure describes a method for performing mass spectrometry, comprising: (a) providing a biological sample comprising one or more peptides and a solvent, wherein the one or more peptides are lysed derivatives of polypeptides adsorbed on a surface; (b) determining an amount of the one or more peptides in the biological sample; (c) drying the biological sample to remove at least a portion of the solvent; (d) reconstituting the biological sample with a buffer, based at least in part on the amount of the one or more peptides, such that the biological sample comprises a predetermined concentration of the one or more peptides; and (e) injecting the biological sample comprising the predetermined concentration of the one or more peptides into a mass spectrometer.

In some embodiments, the predetermined concentration is based at least in part on one or more physicochemical properties of the surface.

In some embodiments, the surface is a particle surface.

In some embodiments, the determining comprises contacting the biological sample with a reagent configured to output a signal, wherein a strength of the signal is correlated with the amount of the one or more peptides in the biological sample.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

In some aspects, the present disclosure provides a method for determining a concentration or an amount of a biomolecule or biomolecule group in a biological sample, the method comprising: (a) contacting the biological sample with a plurality of particle-containing solutions each having a different particle concentration, to generate a plurality of biomolecule coronas each corresponding to an individual solution of the plurality of particle-containing solutions; (b) assaying the plurality of biomolecule coronas for a dataset comprising data corresponding to one or more biomolecules or biomolecule groups comprising the biomolecule or biomolecule group in the biological sample; and (c) determining the concentration or the amount of the biomolecule or the biomolecule group in the biological sample based at least partially on the dataset, wherein the determining is made in the absence of using a reference biomolecule external to the biological sample.

In some embodiments, at least a subset of the plurality of particle-containing solutions differ in particle concentration by at least 1, 2, 3, 4, 5, 6, 7, 8, 9, or 10 orders of magnitude. In some embodiments, at least a subset of the plurality of particle-containing solutions differ in particle concentration by at most 1, 2, 3, 4, 5, 6, 7, 8, 9, or 10 orders of magnitude. In some embodiments, solutions of the plurality of particle-containing solutions have particle concentrations between 1 μg/ml and 100 mg/ml. In some embodiments, solutions of the plurality of particle-containing solutions have particle concentrations of at least 1, 2, 3, 4, 5, 6, 7, 8, 9, 10, 20, 30, 40, 50, 60, 70, 80, 90, 100, 200, 300, 400, 500, 600, 700, 800, or 900 μg/ml. In some embodiments, solutions of the plurality of particle-containing solutions have particle concentrations of at most 1, 2, 3, 4, 5, 6, 7, 8, 9, 10, 20, 30, 40, 50, 60, 70, 80, 90, 100, 200, 300, 400, 500, 600, 700, 800, or 900 μg/ml. In some embodiments, solutions of the plurality of particle-containing solutions have particle concentrations of at least 1, 2, 3, 4, 5, 6, 7, 8, 9, 10, 20, 30, 40, 50, 60, 70, 80, 90, or 100 mg/ml. In some embodiments, solutions of the plurality of particle-containing solutions have particle concentrations of at most 1, 2, 3, 4, 5, 6, 7, 8, 9, 10, 20, 30, 40, 50, 60, 70, 80, 90, or 100 mg/ml. In some embodiments, the plurality of biomolecule coronas are associated with a single particle type in solutions of the plurality of particle-containing solutions. In some embodiments, each of the particle-containing solutions comprises a same particle type. In some embodiments, each of the particle-containing solutions comprises a different particle type. In some embodiments, each of the particle-containing solutions comprises a same particle panel comprising a plurality of different particles.

In some embodiments, particles in an individual solution of the plurality of particle-containing solutions have a polydispersity of less than 1 or 0.5. In some embodiments, particles in an individual solution of the plurality of particle-containing solutions have a polydispersity of greater than 1 or 0.5. In some embodiments, the polydispersity is determined at least in part by size variance of the particles. In some embodiments, the polydispersity is determined at least in part by mass variance of the particles.

In some embodiments, a solution of the plurality of particle-containing solutions comprises a surface modified particle. In some embodiments, a solution of the plurality of particle-containing solutions comprises a plurality of surface modified particles. In some embodiments, the plurality of surface modified particles comprises particles having different physicochemical properties. In some embodiments, the physicochemical properties comprise size, charge, core material, shell material, porosity, density, hydrophobicity, hydrophilicity, charge, rigidity, or any combination thereof.

In some embodiments, the dataset comprises a plurality of signals corresponding to the plurality of biomolecule coronas. In some embodiments, the plurality of signals comprises optical signals, electrical signals, or a combination thereof. In some embodiments, the dataset comprises a plurality of datasets. In some embodiments, the determining of (c) comprises comparing intensities of the plurality of signals against an intensity of a reference signal. In some embodiments, the reference signal is associated with a biomolecule intrinsic to the sample. In some embodiments, the biomolecule intrinsic to the sample comprises albumin, globulin, transferrin, fibrinogen, antitrypsin, al-acid glycoprotein, apolipoprotein, ceruloplasmin, transthyretin, a complement factor, or any combination thereof. In some embodiments, the dataset comprises training data for a machine learning algorithm.

In some embodiments, the contacting of (a) for each of the plurality of particle-containing solutions is for about a same duration of time. In some embodiments, the same duration of time is shorter than the equilibration times of the plurality of particle-containing solutions subsequent to the contacting of (a). In some embodiments, the contacting the biological sample with the plurality of particle-containing solutions comprises combining at most about 250 μL of the biological sample with at most about 250 μL of a particle-containing solution of the plurality of particle-containing solutions. In some embodiments, the contacting the biological sample with the plurality of particle-containing solutions comprises combining at most about 100 μL of the biological sample with at most about 100 μL of a particle-containing solution of the plurality of particle-containing solutions. In some embodiments, the contacting the biological sample with the plurality of particle-containing solutions comprises adding at least about 100 nL of plasma per cm² of particle surface area to each solution of the plurality of particle-containing solutions. In some embodiments, the contacting the biological sample with the plurality of particle-containing solutions comprises adding between about 100 nL and 100 mL of plasma per cm² of particle surface area to each solution of the plurality of particle-containing solutions.

In some embodiments, the one or more biomolecules or biomolecule groups comprise a plurality of biomolecules or biomolecule groups, and wherein the determining of (c) comprises identifying a concentration or an amount of each of the plurality of biomolecules or biomolecule groups in the biological sample. In some embodiments, the concentrations of the plurality of biomolecules or biomolecule groups are identified in a single assay. In some embodiments, the biomolecule or biomolecule group comprises a protein or protein group. In some embodiments, the concentration of the biomolecule or the biomolecule group is less than about 10 μg/ml, 1 μg/ml, 100 ng/ml, 10 ng/ml, 1 ng/ml, or 100 μg/ml. In some embodiments, the concentration of the biomolecule or the biomolecule group is greater than about 10 μg/ml, 1 μg/ml, 100 ng/ml, 10 ng/ml, 1 ng/ml, or 100 μg/ml. In some embodiments, the biomolecule or biomolecule group comprises a plurality of human plasma proteins or human plasma protein groups, and wherein the plurality of human plasma proteins or human plasma protein groups comprises at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 proteins or protein groups. In some embodiments, the biomolecule or biomolecule group comprises a plurality of human plasma proteins or human plasma protein groups, and wherein the plurality of human plasma proteins or human plasma protein groups comprises at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 proteins or protein groups. In some embodiments, the determining of (c) comprises determining concentrations of the at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 proteins or protein groups based at least partially on intensities of the plurality of signals. In some embodiments, the determining of (c) comprises determining concentrations of the at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 proteins or protein groups based at least partially on intensities of the plurality of signals. In some embodiments, the determining of (c) comprises identifying relative abundances of a plurality of isoforms of a protein.

In some embodiments, the assaying of (b) comprises separating the plurality of biomolecule coronas from the biological sample. In some embodiments, the separating comprises magnetically separating the plurality of biomolecule coronas from the biological sample. In some embodiments, the assaying of (b) comprises digesting the one or more biomolecules or biomolecule groups.

In some embodiments, a particle concentration of the plurality of the particle-containing solutions is approximately equal to a total protein concentration of the biological sample. In some embodiments, the biological sample is diluted by at least 2, 3, 4, 5, 6, 7, 8, 9, or 10-fold prior to the contacting with the plurality of particle-containing solutions. In some embodiments, the biological sample is diluted by at most 2, 3, 4, 5, 6, 7, 8, 9, or 10-fold prior to the contacting with the plurality of particle-containing solutions.

In some embodiments, particles of the plurality of particle-containing solutions have diameters between about 100 and about 500 nanometers. In some embodiments, particles of the plurality of particle-containing solutions have diameters between about 100 and about 300 nanometers. In some embodiments, particles of the plurality of particle-containing solutions comprise diameters of at least about 500 nanometers. In some embodiments, particles of the plurality of particle-containing solutions comprise diameters of at most about 200 nanometers.

In some embodiments, the plurality of particle-containing solutions comprises a particle selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. In some embodiments, the plurality of particle-containing solutions comprises at least two particles selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. In some embodiments, the plurality of particle-containing solutions comprises at least three particles selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. In some embodiments, the plurality of particle-containing solutions comprises at least four particles selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. In some embodiments, the plurality of particle-containing solutions comprises a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. In some embodiments, the plurality of particle-containing solutions comprises a superparamagnetic iron oxide particle (SPION) comprising a poly(N-(3-(dimethylamino) propyl) methacrylamide) (PDMAPMA) surface. In some embodiments, the plurality of particle-containing solutions comprises a superparamagnetic iron oxide particle (SPION) comprising a poly(oligo (ethylene glycol) methyl ether methacrylate) (POEGMA) surface. In some embodiments, the plurality of particle-containing solutions comprises a superparamagnetic iron oxide particle (SPION) comprising an N-(3-Trimethoxysilylpropyl) diethylenetriamine surface. In some embodiments, the plurality of particle-containing solutions comprises a superparamagnetic iron oxide particle (SPION) comprising a Poly(dimethyl aminopropyl methacrylamide) (Dimethylamine) surface. In some embodiments, the plurality of particle-containing solutions comprises a superparamagnetic iron oxide particle (SPION) comprising a dextran surface. In some embodiments, the plurality of particle-containing solutions comprises a superparamagnetic iron oxide particle (SPION) comprising a surface with a mixed chemistry based on amine-epoxy chemistry. In some embodiments, the plurality of particle-containing solutions comprises a superparamagnetic iron oxide particle (SPION) comprising a Polyzwitterion coated (Poly(N-[3-(Dimethylamino) propyl]methacrylamide-co-[2-(methacryloyloxy)ethyl]dimethyl-(3-sulfopropyl) ammonium hydroxide, P (DMAPMA-co-SBMA)) surface. In some embodiments, the plurality of particle-containing solutions comprises a superparamagnetic iron oxide particle (SPION) comprising styrene surface comprising an oleic acid functionalization. In some embodiments, the plurality of particle-containing solutions comprises a superparamagnetic iron oxide particle (SPION) comprising a boronated styrene surface. In some embodiments, the plurality of particle-containing solutions comprises a superparamagnetic iron oxide particle (SPION) comprising a carboxylated styrene surface. In some embodiments, the plurality of particle-containing solutions comprises a superparamagnetic iron oxide microparticle (SPION) comprising a carboxylated styrene surface. In some embodiments, the plurality of particle-containing solutions comprises a superparamagnetic iron oxide microparticle (SPION) comprising a strongly acidic silica surface.

In some embodiments, the dataset comprises a plurality of factors or a plurality of functions that account for the differences between one or more amounts of the one or more biomolecules or biomolecule groups in the one or more biomolecule coronas. In some embodiments, the plurality of factors or the plurality of functions are specific to the particle. In some embodiments, the plurality of factors or the plurality of functions are specific to the biomolecule or the biomolecule group.

In some embodiments, the concentration or the amount of the biomolecule or the biomolecule group is correlated with an intrinsic concentration or an intrinsic amount of the biomolecule or the biomolecule group measured from the biological sample without contacting with a particle-containing solution, with a Pearson correlation coefficient of at least 0.1, 0.2, 0.3, 0.4, 0.5, 0.6, 0.7, 0.8, or 0.9. In some embodiments, the concentration or the amount of the biomolecule or the biomolecule group is correlated with an intrinsic concentration or an intrinsic amount of the biomolecule or the biomolecule group measured from the biological sample without contacting with a particle-containing solution, with a Pearson correlation coefficient of at most 0.1, 0.2, 0.3, 0.4, 0.5, 0.6, 0.7, 0.8, 0.9, or 1.0.

In some aspects, the present disclosure provides a method for determining a concentration or an amount of a plurality of protein groups in a biological sample, the method comprising: (a) contacting a reference biological sample with (i) a first particle-containing solution comprising a first concentration of a particle to generate a first protein corona and (ii) a second particle-containing solution comprising a second concentration of the particle to generate a second protein corona, wherein the first concentration is higher than the second concentration; (b) performing mass spectrometry using (i) the first protein corona to determine a first plurality of protein group intensities of the plurality of protein groups in the first protein corona and (ii) the second protein corona to determine a second plurality of protein group intensities of the plurality of protein groups in the second protein corona; (c) determining a plurality of factors or a plurality of functions that account for the differences between the first plurality of protein group intensities and the second plurality of protein group intensities, wherein each of the plurality of factors or the plurality of functions are specific to the particle and to each protein group in the plurality of protein groups; (d) contacting the biological sample with a third particle-containing solution comprising a third concentration of the particle to generate a third protein corona, wherein the third concentration is less than or equal to about the first concentration, and greater than or equal to about the second concentration; (e) assaying the third protein corona to determine a third plurality of protein group intensities of the plurality of protein groups in the third protein corona; and (f) applying the plurality of factors or the plurality of functions to the plurality of protein group intensities to determine a fourth plurality of protein group intensities, such that the Pearson correlation coefficient is at least 0.5 between the fourth plurality of protein group intensities and intensities of the protein groups measured by performing mass spectrometry on the biological sample without contacting with a particle-containing solution.

In some aspects, the present disclosure provides a method for performing mass spectrometry, comprising: (a) providing a biological sample comprising one or more peptides and a solvent, wherein the one or more peptides comprise proteolytically cleaved derivatives of proteins adsorbed on a surface; (b) determining an amount of the one or more peptides in the biological sample; (c) drying the biological sample to remove at least a portion of the solvent; (d) reconstituting the biological sample with a second solvent, based at least in part on the amount of the one or more peptides, such that the biological sample comprises a predetermined concentration of the one or more peptides; and (e) assaying the biological sample. In some embodiments, the method further comprises performing (a)-(e) for a second plurality of peptides in serial or in parallel. In some embodiments, the one or more peptides comprise a plurality of peptides, and the assaying comprises determining a relative amount between at least two peptides in the plurality of peptides. In some embodiments, the drying comprises drying using vacuum.

In some embodiments, the surface comprises a sensor element surface. In some embodiments, the sensor element surface comprises a particle surface. In some embodiments, the particle surface is a nanoparticle surface. In some embodiments, the particle surface is a microparticle surface. In some embodiments, the particle surface comprises pores. In some embodiments, the proteins are bound on the surface via adsorption. In some embodiments, the proteins are bound on the surface via non-specific binding. In some embodiments, the proteins are bound on the surface via specific binding. In some embodiments, the proteins form a corona on the particle surface. In some embodiments, the predetermined concentration is based at least in part on one or more physicochemical properties of the surface. In some embodiments, the one or more physicochemical parameters comprise: sample to surface ratio, incubation time, pH, salt concentration, ionic strength, solvent composition, solvent dielectric constant, crowding agent concentration, temperature, sample composition, surfactant concentration, concentration of enzymes, activity of enzymes, chemical reactions, concentrations of small molecules, surface chemistry, or any combination thereof.

In some embodiments, the determining comprises contacting the biological sample with a reagent configured to output a signal, wherein a strength of the signal is correlated with the amount of the one or more peptides in the biological sample. In some embodiments, the reagent comprises a fluorescing reagent and the signal comprises a fluorescent signal.

In some embodiments, the method further comprises, prior to (a), proteolytically cleaving the proteins to generate the one or more peptides. In some embodiments, the method further comprises, prior to proteolytically cleaving, contacting the proteins with the surface. In some embodiments, proteolytically cleaving comprises contacting the proteins with trypsin, lysin, or both.

In some embodiments, the assaying comprises mass spectrometry. In some embodiments, the mass spectrometry comprises liquid-chromatography tandem mass spectrometry (LC-MS/MS). In some embodiments, the assaying comprises protein sequencing. In some embodiments, the assaying comprises binding each protein in the proteins to a pair of antibodies. In some embodiments, the pair of antibodies comprises complementary single-stranded nucleic acid sequences attached thereto, such that when the pair of antibodies bind to the molecule, the complementary nucleic acids hybridize to form a double stranded nucleic acid. In some embodiments, the double stranded nucleic acid is configured to form a binding complex with a polymerase and a plurality of nucleotides, nucleosides, nucleotide analogs, and/or nucleoside analogs to perform an amplification reaction to produce a detectable signal. In some embodiments, the assaying comprises binding a protein in the proteins to an aptamer. In some embodiments, the assaying comprises an immunoassay.

In some embodiments, the biological sample is derived from a complex biological sample. In some embodiments, the biological sample is derived from plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, the biological sample is derived from plasma or serum.

In some aspects, the present disclosure provides a method for performing mass spectrometry, comprising: (a) providing a substrate comprising a plurality of wells or chambers, wherein the plurality of wells or chambers comprises: (i) a first well or chamber comprising a first biological sample therein, wherein the first biological sample comprises a first set of peptides and a first solvent, wherein the first set of peptides comprises proteolytically cleaved derivatives of a first set of proteins adsorbed on a first surface; and (ii) a second well or chamber comprising a second biological sample therein, wherein the second biological sample comprises a second set of peptides and a second solvent, wherein the second set of peptides comprises proteolytically cleaved derivatives of a second set of proteins adsorbed on a second surface; (b) determining (i) a first amount of the first set of peptides in the first biological sample and (ii) a second amount of the second set of peptides in the second biological sample; (c) drying (i) the first biological sample to remove at least a portion of the first solvent and (ii) the second biological sample to remove at least a portion of the second solvent; (d) reconstituting (i) the first biological sample with a first buffer based at least in part on the first amount and (ii) the second biological sample with a second buffer based at least in part on the second amount, such that the first biological sample and the second biological sample comprises about a predetermined concentration of peptides; (e) injecting (i) the first biological sample into a mass spectrometer to generate a first set of peptide intensities and (ii) the second biological sample into the mass spectrometer to generate a second set of peptide intensities; and (f) generating a dataset comprising the first set of peptide intensities and the second set of peptide intensities, wherein a bias arising from differences in input concentration of peptides into the mass spectrometer is normalized between the first set of peptide intensities and the second set of peptide intensities, such that the first set of peptide intensities and the second set of peptide intensities are proportional to a common reference without further renormalization.

In some aspects, the present disclosure provides a method for performing mass spectrometry, comprising: (a) providing a first biological sample comprising a first set of peptides and a first solvent, wherein the first set of peptides comprises proteolytically cleaved derivatives of a first set of proteins adsorbed on a first surface; (b) determining a first amount of the first set of peptides in the first biological sample; (c) drying the first biological sample to remove at least a portion of the first solvent; (d) reconstituting the first biological sample with a first buffer based at least in part on the first amount, such that the first biological sample comprises about a predetermined concentration of peptides; (e) injecting the first biological sample into a mass spectrometer to generate a first set of peptide intensities; (f) providing a second biological sample comprising a second set of peptides and a second solvent, wherein the second set of peptides comprises proteolytically cleaved derivatives of a second set of proteins adsorbed on a second surface; (g) determining a second amount of the second set of peptides in the second biological sample; (h) drying the second biological sample to remove at least a portion of the second solvent; (i) reconstituting the second biological sample with a second buffer based at least in part on the second amount, such that the second biological sample comprises about the predetermined concentration of peptides; (j) injecting the second biological sample into a mass spectrometer to generate a second set of peptide intensities; and (k) generating a dataset comprising the first set of peptide intensities and the second set of peptide intensities, wherein a bias arising from differences in input concentration of peptides into the mass spectrometer is normalized between the first set of peptide intensities and the second set of peptide intensities, such that the first set of peptide intensities and the second set of peptide intensities are proportional to a common reference without further renormalization.

In some aspects, the present disclosure provides a computer program product comprising a computer-readable medium having computer-executable code encoded therein, the computer-executable code adapted to be executed to implement any one of the methods disclosed herein.

In some aspects, the present disclosure provides a non-transitory computer-readable storage media encoded with a computer program including instructions executable by one or more processors to implement any one of the methods disclosed herein.

In some aspects, the present disclosure provides a computer-implemented system comprising: a digital processing device comprising: at least one processor, an operating system configured to perform executable instructions, a memory, and a computer program including instructions executable by the digital processing device to perform any one of the methods of disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 provides a plot showing the dependence of particle corona content on sample dilution. The plot provides data from dilution assays in which five different types or volumes of particles were contacted with five different volumes of a sample, and displays the total protein adsorbed onto each type of particle at each dilution level.

FIG. 2 shows the quantities of different proteins adsorbed to a particle from solutions having undergone different degrees of dilution. A complex protein sample was diluted at factors of 1, 2.5, 5, 10 and 20-fold, and then contacted to a set of carboxyl functionalized polystyrene nanoparticles. The total amount of each type of protein collected on the particles was quantified by LCMS. Each trace on the plot corresponds to a unique type of protein, and provides its LCMS intensity as a function of sample dilution.

FIG. 3 shows the results of a proteomics assay involving protein collection on nanoparticles. Protein binding was interrogated for 5 different types of nanoparticles. Particles were mixed with plasma in 5 different volume ratios. The graph shows the total amount of protein collected on each particle at its respective mixing volume ratio.

FIG. 4 shows intersection sizes for the protein adsorption dependence data in FIG. 3.

FIG. 5 provides aggregate protein adsorption data onto 5 different types of particles. Panel A displays the mass of protein adsorbed onto particular types at specific plasma-to-particle mixing volumes. Panel B provides the data from panel A plotted as a function of nanoparticle input volume.

FIG. 6 provides results from an assay in which protein coronas were formed on five types of particles at five separate plasma-to-particle mixing volumes. Panel A displays the number of distinct protein groups adsorbed in each assay. Panel B displays the total mass of protein adsorbed in each assay.

FIG. 7 shows the coefficients of variation (CV) for the abundances of the protein groups in FIG. 6.

FIG. 8 provides results from an experiment in which human plasma samples were combined in five different volume ratios with a sample containing five types of particles. Panel A shows the total number of proteins and distinct protein groups collected in each mixture. Panel B provides the protein group data from panel A, plotted as a function of normalized nanoparticle concentration. Panel C provides the protein group data from panel A, plotted as a function of the plasma-to-particle ratio in each mixture.

FIG. 9 provides results from a simulation of particle-solute interaction strength in which 300 nm particles were modeled as univalent hard spheres surrounded by small ions. Panel A displays calculated double layer force as a function particle-ion distance and ion concentration. Panel B graphically illustrates the types of solute spheres surrounding the particle.

FIG. 10 shows titration curves for multiple types of particles.

FIG. 11 provides Langmuir adsorption isotherms for particles contacted by a range of samples with different protein concentrations. Panels A and B depict two distinct saturation behaviors.

FIG. 12 graphically illustrates a series of protein-particle binding calculations, based on the equilibrium binding equation qₑ=((C₀-Cₑ)*V)/m, where qₑ is equilibrium adsorption (mass protein adsorbed per mass of particle), C₀ is initial protein concentration, Cₑ is equilibrium protein concentration, V is sample volume, and m is particle mass.

FIG. 13 provides a heatmap for protein binding to various carboxylate and amine functionalized particle-types.

FIG. 14 shows pH dependent binding data to 8 types of particles for three types of proteins. Panel A shows results for pregnancy zone protein, pI 5.91. Panel B shows results for proteoglycan 4, pI 9.53. Panel C shows results for cartilage oligomeric matrix protein (COMP), pI 4.37.

FIG. 15 provides time-dependent protein corona compositional data. Panel A shows the number of types of proteins bound to 5 different nanoparticles at 5 different times following sample-particle mixing. Panel B shows the overlap in the types of protein at three separate timepoints for a carboxylate functionalized nanoparticle.

FIG. 16 shows corona composition dependence on buffer-type for 5 different particles.

FIG. 17 illustrates possible effects from changing salt type and salt concentration on protein solubility and protein adsorption to sensor elements.

FIG. 18 depicts the structures of 6 types of functionalized superparamagnetic iron oxide nanoparticles (SPIONs).

FIG. 19 shows transmission electron microscopy (TEM) images of three types of SPIONs.

FIG. 20 shows TEM images of three polymeric nanoparticles.

FIG. 21 illustrates a method for capturing proteins on particles and analyzing the particles with mass spectrometry.

FIG. 22A provides the number of types of protein groups collected on carboxyl functionalized polystyrene particles (NP-A) at different concentrations.

FIG. 22B provides the number of types of protein groups collected on poly(dimethylaminopropylmethacrylamide) particles (NP-E) at different concentrations.

FIG. 22C depicts the amount of overlap between the types of proteins identified on two particle types at multiple concentrations and the types of proteins identified from neat plasma samples.

FIG. 23 depicts early (panel A) and late (panel B) timepoints in biomolecule corona formation, illustrating a change in biomolecules adsorbed to a particle over time.

FIG. 24 presents protein group identification numbers obtained with a range of plasma-to-particle ratios for NP-C (panel A), NP-D (panel B), NP-E (panel C), NP-A (panel D), NP-B (panel E) and the 5-particle panel (panel F).

FIG. 25 provides Jaccard Similarity Coefficients (JI) for assay replicates at a range of particle concentrations for NP-D (Panel A), NP-E (Panel B), NP-A (Panel C), and NP-B (Panel D) particles.

FIG. 26 provides coefficient of variation (CV) values for the protein groups identified in neat plasma (panel A) and with NP-D (Panel B), NP-E (Panel C), NP-A (Panel D), and NP-B (Panel E) particles.

FIG. 27 provides coefficient of variation (CV) values for protein groups commonly identified on NP-D, NP-E, NP-A, and NP-B particles for a range of particle concentrations.

FIG. 28 provides CV accumulation curves for NP-A (Panel A), and NP-B (Panel B), NP-D (Panel C) and NP-E (Panel D) particles, with each curve representing a different particle concentration.

FIG. 29 provides protein group identification numbers for a variety of particle panels as a function of particle panel size.

FIG. 30 provides CV accumulation curves for protein group identifications with a low concentration of a two particle panel (NP-E and NP-A), a moderate concentration of a four particle panel (NP-D, NP-E, NP-A and NP-B), and direct analysis of neat plasma.

FIG. 31 provides percent coverage of Carr database (Keshishian et al., Mol. Cell Proteomics 14, 2375-2393 (2015)) proteins as a function of protein abundance for the low concentration of the two particle panel (NP-E and NP-A), the moderate concentration of the four particle panel (NP-D, NP-E, NP-A and NP-B), and the neat plasma analysis of FIG. 30.

FIG. 32 illustrates protein group identification numbers obtained with varying concentrations of NP-E and NP-A particles.

FIG. 33 provides correlation coefficients between the sets of protein groups identified in neat plasma and the sets of protein groups identified on NP-A (panel A), NP-B (panel B), NP-D (panel C) and NP-E (panel D) particles.

FIG. 34 provides a schematic overview of biomolecule formation following contact between a biological sample and a particle panel.

FIG. 35 provides a sample workflow for a particle-based biomolecule corona assay.

FIG. 36 outlines steps for a sample particle-based biomolecule corona assay.

FIG. 37 provides protein group identification numbers for particle panels of varying size.

FIG. 38 shows a computer system that is programmed or otherwise configured to implement methods provided herein.

FIG. 39A-I provides protein group identifications obtained through biomolecule corona analysis with a range of particles. FIG. 39A provides data obtained with a silica-coated superparamagnetic iron oxide nanoparticle (SPION). FIG. 39B provides data obtained with a poly(dimethylaminopropylmethacrylamide)-coated SPION. FIG. 39C provides data obtained with a 1,6-hexanediamine-coated SPION. FIG. 39D provides data obtained with a mixed amide, carboxylate functionalized, silica-coated SPION. FIG. 39E provides data obtained with a N¹-(3-(trimethoxysilyl) propyl) hexane-1,6-diamine functionalized, silica-coated SPION. FIG. 39F provides data obtained with a carboxyl functionalized polystyrene-coated SPION. FIG. 39G provides data obtained with a dextran-coated SPION. FIG. 39H provides data obtained with a particle panel comprising a silica-coated SPION, a poly(dimethylaminopropylmethacrylamide)-coated SPION, an N-(3-Trimethoxysilylpropyl) diethylenetriamine-coated SPION, a carboxyl functionalized polystyrene-coated SPION, and a dextran-coated SPION. FIG. 39I provides data obtained with a particle panel comprising a silica-coated SPION, a poly(dimethylaminopropylmethacrylamide)-coated SPION, an N-(3-Trimethoxysilylpropyl) diethylenetriamine-coated SPION, a 1,6-hexanediamine-coated SPION, and an N¹-(3-(trimethoxysilyl) propyl) hexane-1,6-diamine functionalized, silica-coated SPION.

FIG. 40A-B show PCA (principal component analysis) projections for biomolecules measured from neat plasma, and two nanoparticles at various plasma: nanoparticle ratios using principle component analysis (PCA). FIG. 40C shows PCA projections for biomolecules measured from neat plasma, and two nanoparticles at various plasma: nanoparticle ratios using uniform manifold approximation projection (UMAP).

FIG. 41 shows the correlation coefficient between true biomolecule concentrations in a sample and the biomolecule concentration measured using nanoparticles as a function of plasma: nanoparticle ratios.

FIG. 42 shows a PCA projection for biomolecules measured from neat plasma, and two nanoparticles at various plasma: nanoparticle ratios using principal component analysis (PCA).

FIG. 43 shows a peptide standard quantitation curve, in accordance with some embodiments.

FIG. 44 shows a process diagram for peptide quantitation and reconstitution, in accordance with some embodiments.

FIG. 45 shows an example calculation for peptide quantitation and reconstitution, in accordance with some embodiments.

FIGS. 46A-461 show types of surfaces, in accordance with some embodiments. FIG. 46A illustrates a non-limiting example of a surface functionalized at one or more regions for capturing biomolecules. FIG. 46B illustrates a non-limiting example of a surface comprising one or more wells or depressions for capturing biomolecules. For example, a functionalized surface may be disposed in a 96 well plate or a 384 well plate. FIG. 46C illustrates a non-limiting example of a surface disposed on one or more particles. In some embodiments, the one or more particles may be disposed in one or more wells or depressions. FIG. 46D illustrates anon-limiting example of a surface disposed on a plurality of particles packed in a channel or a porous material disposed in a channel. FIG. 46E illustrates a non-limiting example of a surface disposed on an inner surface of a channel. FIGS. 46F-461 illustrate non-limiting examples of surfaces in accordance with some embodiments of the disclosure. A surface may comprise 1, 2, 3, 4 or any number of distinct surface regions. In some cases, a surface may be disposed on a particle. In some cases, a particle may be a porous particle.

### DETAILED DESCRIPTION

Introducing a nanoparticle (NP) or other surfaces into a biofluid, such as blood plasma, can lead to the formation of a selective and reproducible protein corona at the nano-bio interface driven by a combination of protein-surface affinity, protein abundance, and protein-protein interactions. These interactions can be exploited to interrogate the entire plasma proteome at scale and depth without the inherent bias of targeted analyte-specific probes (e.g., antibodies or aptamers). When introduced into a biological matrix, proteins may assemble on surfaces to forma protein corona via physical adsorption and/or electrostatic interactions. Without requiring a presence of a specific entity that is configured for binding to a singular specific protein (e.g., as in immunoassays), the nanoparticles can allow dynamic range compression of proteins bound to the nanoparticle surfaces while capturing a wide variety of proteins. In other words, the relative abundance of proteins in the sample can be modified on the nanoparticle surfaces, such that the rare proteins are relatively more abundant, and the highly abundant proteins are relatively less abundant compared to the original sample.

At preequilibrium, the protein corona composition can be driven by the relative proximity of proteins that diffuse to interacting moieties on the particle surface. As such, proteins with high abundance can dominate the initial corona composition. At equilibrium, governed by thermodynamics, high-abundance low-affinity proteins on the NP surface can be displaced by low-abundance high-affinity proteins (Vroman effect), which may lead to compression of the dynamic range. The competition between proteins for binding to a surface (e.g., the Vroman effect) can play an important role in protein corona composition, and surfaces can be tuned with different functionalizations to enhance and differentiate protein selectivity. The quantitative composition of protein coronas thus can depend on the physicochemical properties of the surfaces, the presence and abundance of proteins with compatible surface epitopes, and the competition of proteins for binding.

The compression of the dynamic range can confer significant advantages in determining the biomolecule composition in biofluids such as human plasma. Human plasma contains protein species over a dynamic range that exceeds 12 orders of magnitude, where the top few proteins (e.g., albumin, transferrin, complement proteins, apolipoproteins, and alpha-2-macroglobulin) comprise 95% of the mass of protein in the plasma, and most of the protein species comprise the remaining 5%. Some of the protein species exist in the nanograms per milliliter ranges (e.g., transforming growth factor beta-1-induced transcript 1 protein at ˜10 ng/ml; fructose-bisphosphate aldolase A at ˜20 ng/ml; thioredoxin at ˜18 ng/ml; and L-selectin at ˜92 ng/ml), and some proteins are expected to present at level even beneath that range. Liquid chromatography coupled with mass spectrometry (LC-MS) or tandem mass spectrometry (LC-MS/MS) can be used to identify protein species in plasma; however, due to the stochastic nature of the methods, only a fraction of ionic species that are generated at a time from a given sample may be selected for acquiring mass spectra. As a result, the species that are highly abundant compared to the rare species can generate a signal that overwhelms signal from rare species. Compressing the dynamic range of protein species in a sample can allow rare proteins to comprise a higher fraction of ionic species, thereby allowing higher probability for detecting those rare proteins in a MS experiment. This process, incorporated within the Proteograph™ proteomics platform, may offer superior plasma profiling performance in terms of depth and breadth, compared to conventional shallow and deep workflows.

Protein corona formation can be a complex process that can be governed by a large number of interrelated variables. Various aspects of the present disclosure provide methods for obtaining or otherwise estimating quantities of proteins in a sample before the dynamic range compression using nanoparticles. In some aspects, the present disclosure provides a process which can comprise measuring quantities of proteins using nanoparticles to compress the dynamic range, and then decompressing the measured quantities to the quantities that are expected in the sample before dynamic range compression. In some cases, biomolecule corona formation can be affected or controlled by modifying sample conditions. For example, biomolecule corona formation can be affected by diluting a sample, by adjusting the aggregate surface area of sensor elements in a sample, or varying solution conditions (e.g., salt concentration, pH, or temperature).

In some aspects, the present disclosure provides a method for determining a concentration or an amount of a plurality of protein groups in a biological sample. As discussed above, the amount of a biomolecule detected using a particle can comprise some amount of bias associated with the kinetics and the thermodynamics of binding. In some aspects, the method can be useful in accounting for at least some of the bias in order to obtain a more accurate measure of the concentration or the amount of a protein group in the biological sample.

The method can comprise taking two or more measurements at different particle concentrations. For example, the method can comprise contacting a reference biological sample with a first particle-containing solution comprising a first concentration of a particle to generate a first protein corona. The method can comprise contacting the reference biological sample with a second particle-containing solution comprising a second concentration of the particle to generate a second protein corona. The first concentration may be higher than the second concentration, or vice versa. The difference in the concentrations can be at least 2, 3, 4, 5, 6, 7, 8, 9, or 10-fold. The difference in the concentrations can be at most 2, 3, 4, 5, 6, 7, 8, 9, or 10-fold. The different concentrations can be represented as a ratio between the mass, volume, or surface area of the particle and the mass or volume of the biological sample. Each of the plurality of particle-containing solutions can be contacted with the reference biological sample for the same duration of time, or different durations of time. The duration of time can be shorter or longer than the equilibrium times of the plurality of particle-containing solutions during the contact. The equilibrium time can be the time it takes for binding events between biomolecules and surfaces in a particle-containing solution to reach equilibrium. The bias associated with a measurement can become larger as the particle concentration decreases, although rarer protein groups can be detected at lower particle concentrations. The method can comprise performing mass spectrometry using the first protein corona to determine a first plurality of protein group intensities of the plurality of protein groups in the first protein corona. The method can comprise performing mass spectrometry using the second protein corona to determine a second plurality of protein group intensities of the plurality of protein groups in the second protein corona. The method can comprise determining intensities of a plurality of isoforms of a protein in the first protein corona. The method can comprise determining intensities of a plurality of isoforms of a protein in the second protein corona.

Using the measurements taken at different particle concentrations, one can derive a factor or a function that can be applied to a measurement taken at a higher particle concentration in order to obtain a more accurate measure of the concentration or the amount of a protein group in the biological sample. The method can comprise determining a plurality of factors or a plurality of functions that account for the differences between the first plurality of protein group intensities and the second plurality of protein group intensities. Each of the plurality of factors or the plurality of functions can be specific to the particle and/or to each protein group in the plurality of protein groups. The plurality of factors or plurality of functions can then be applied to another sample to obtain the more accurate measure. The method can comprise contacting the biological sample with a third particle-containing solution comprising a third concentration of the particle to generate a third protein corona. The third concentration can be less than or equal to about the first concentration, and/or be greater than or equal to about the second concentration. The method can comprise assaying the third protein corona to determine a third plurality of protein group intensities of the plurality of protein groups in the third protein corona. The method can comprise applying the plurality of factors or the plurality of functions to the plurality of protein group intensities to determine a fourth plurality of protein group intensities. The Pearson correlation coefficient can be at least 0.5 between the fourth plurality of protein group intensities and a reference signal. In some cases, the reference signal can be associated with a biomolecule intrinsic to the sample. The Pearson correlation coefficient can be at least 0.5 between the fourth plurality of protein group intensities and intensities of the protein groups measured by performing mass spectrometry on the biological sample without contacting with a particle-containing solution. In some cases, the Pearson correlation coefficient can be at least 0.6, 0.7, 0.8, 0.9, or 0.95. In some cases, the Pearson correlation coefficient can be at most 0.6, 0.7, 0.8, 0.9, 0.95, or 1.

In some aspects, the present disclosure provides a method for performing mass spectrometry that can allow sample to sample comparison of MS intensities. For instance, when particles with different surface chemistries are used to compress the dynamic range of biomolecules in a sample, keeping the amount of biomolecules injected into a mass spectrometer consistent between different particles can improve sample to sample comparison of MS intensities. Even when particles with the same surface chemistries are used, keeping the amount of biomolecules injected into a mass spectrometer consistent between different particles can improve sample to sample comparison of MS intensities. The amount of biomolecules injected can be kept consistent in parallel (e.g., corona compression may be performed in parallel on the same 96-well plate before injecting biomolecules into a mass spectrometer) or in series (e.g., corona compression may be performed in series on different 96-well plates before injecting biomolecules into a mass spectrometer).

The method can comprise providing a substrate comprising a plurality of wells or chambers. The plurality of wells or chambers can comprise a first well or chamber comprising a first biological sample therein. The first biological sample can comprise a first set of peptides and a first solvent. The first set of peptides can comprise proteolytically cleaved derivatives of a first set of proteins adsorbed on a first surface. The plurality of wells or chambers can comprise a second well or chamber comprising a second biological sample therein. The second biological sample can comprise a second set of peptides and a second solvent. The second set of peptides can comprise proteolytically cleaved derivatives of a second set of proteins adsorbed on a second surface. The first and the second solvent can be a solvent that was originally in the first biological sample, e.g., water, or those that were added, e.g., buffers. The peptides can have been proteolytically cleaved by a protease, e.g., trypsin or lysin.

The method can comprise determining a first amount of the first set of peptides in the first biological sample. The method can comprise determining a second amount of the second set of peptides in the second biological sample. The method can comprise drying the first biological sample to remove at least a portion of the first solvent. The method can comprise drying the second biological sample to remove at least a portion of the second solvent. The drying can comprise applying negative pressure (e.g., negative gauge pressure with respect to atmospheric pressure), while optionally heating or chilling the drying sample. The drying may proceed to the extent until solvent evaporation is no longer observable, e.g., through changes in mass. The drying can be performed for at least 1, 2, 3, 4, 5, 6, 7, 8, 9, 10, 20, 30, 40, 50, or 60 minutes. The drying can be performed for at least 1, 2, 3, 4, 5, 6, 7, 8, 9, 11, 12, 24, 36, or 48 hours. The drying can be performed for at most 1, 2, 3, 4, 5, 6, 7, 8, 9, 10, 20, 30, 40, 50, or 60 minutes. The drying can be performed for at most 1, 2, 3, 4, 5, 6, 7, 8, 9, 11, 12, 24, 36, or 48 hours. The drying can be performed at about room temperature. The drying can be performed at a temperature of at least-200, −150, −100, −50, −25, 0, 25, 50, 75, or 100° C. The drying can be performed at a temperature of at most-200, −150, −100, −50, −25, 0, 25, 50, 75, or 100° C. The drying can be performed at a negative gauge pressure of at least 10, 20, 30, 40, 50, 60, 70, 80, 90, or 100 kilopascals (kPa). The drying can be performed at a negative gauge pressure of at most 10, 20, 30, 40, 50, 60, 70, 80, 90, or 100 kPa.

The method can comprise reconstituting the first biological sample with a first buffer based at least in part on the first amount. The method can comprise reconstituting the second biological sample with a second buffer based at least in part on the second amount. The first and the second buffer can be the same or different. The first biological sample and the second biological sample, when reconstituted, can comprise about a predetermined concentration of peptides. The predetermined concentration can be at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/μL (biomolecule mass/buffer volume). The predetermined concentration can be at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/μL (biomolecule mass/buffer volume). The method can comprise injecting the first biological sample into a mass spectrometer to generate a first set of peptide intensities. The method can comprise injecting the second biological sample into the mass spectrometer to generate a second set of peptide intensities. The method can comprise generating a dataset comprising the first set of peptide intensities and the second set of peptide intensities. A bias arising from differences in input concentration of peptides into the mass spectrometer can be normalized between the first set of peptide intensities and the second set of peptide intensities. The first set of peptide intensities and the second set of peptide intensities can be proportional to a common reference without further renormalization.

In some aspects, the present disclosure provides a method for performing mass spectrometry. The method can comprise providing a first biological sample comprising a first set of peptides and a first solvent. The first set of peptides can comprise proteolytically cleaved derivatives of a first set of proteins adsorbed on a first surface.

The method can comprise determining a first amount of the first set of peptides in the first biological sample. The method can comprise drying the first biological sample to remove at least a portion of the first solvent. The method can comprise reconstituting the first biological sample with a first buffer based at least in part on the first amount. The first biological sample can comprise about a predetermined concentration of peptides. The method can comprise injecting the first biological sample into a mass spectrometer to generate a first set of peptide intensities. The method can comprise providing a second biological sample comprising a second set of peptides and a second solvent. The second set of peptides can comprise proteolytically cleaved derivatives of a second set of proteins adsorbed on a second surface. The method can comprise determining a second amount of the second set of peptides in the second biological sample. The method can comprise drying the second biological sample to remove at least a portion of the second solvent. The method can comprise reconstituting the second biological sample with a second buffer based at least in part on the second amount. The second biological sample can comprise about the predetermined concentration of peptides. The method can comprise injecting the second biological sample into a mass spectrometer to generate a second set of peptide intensities. The method can comprise generating a dataset comprising the first set of peptide intensities and the second set of peptide intensities. A bias arising from differences in input concentration of peptides into the mass spectrometer can be normalized between the first set of peptide intensities and the second set of peptide intensities. The first set of peptide intensities and the second set of peptide intensities can be proportional to a common reference without further renormalization.

### Non-Specific Binding

In some embodiments, a surface binds biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

Non-specific binding may comprise a plurality of non-specific binding affinities for a plurality of targets (e.g., at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.). This may be contrasted with specific binding, which may comprise a higher binding affinity for a given target molecule than non-target molecules.

Biomolecules may adsorb onto a surface through non-specific binding on a surface at various densities. In some cases, biomolecules or proteins may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules or proteins may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm².

Adsorbed biomolecules may comprise various types of proteins. In some cases, adsorbed proteins may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some cases, adsorbed proteins may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins.

In some cases, proteins in a biological sample may comprise at least about 1, 2, 3, 4, 5, 6, 7, 8, 9, 10, 15, 20, 25, or 30 orders of magnitudes in concentration. In some cases, proteins in a biological sample may comprise at most about 1, 2, 3, 4, 5, 6, 7, 8, 9, 10, 15, 20, 25, or 30 orders of magnitudes in concentration.

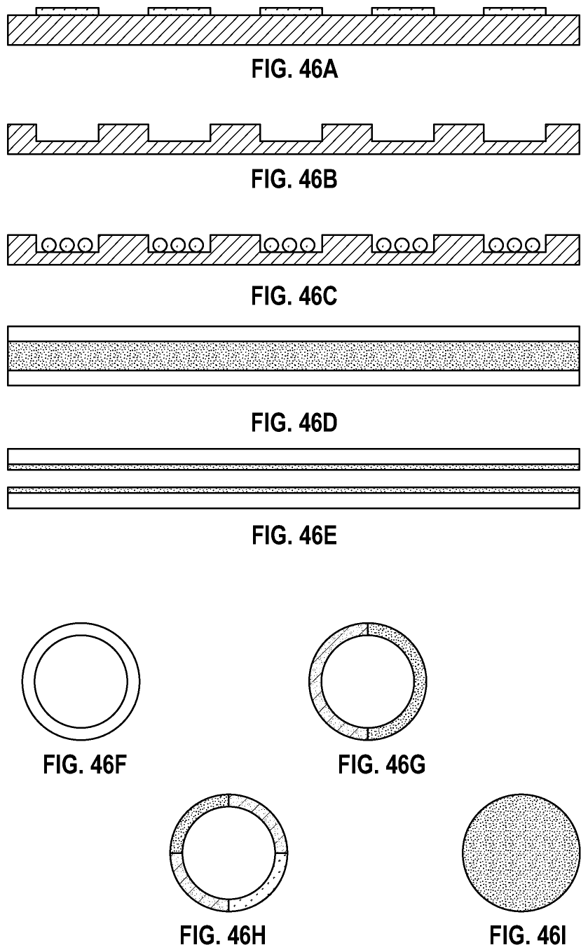

FIG. 46 shows types of surfaces, in accordance with some embodiments. A surface may be functionalized at one or more regions for capturing biomolecules. A surface may comprise one or more wells or depressions for capturing biomolecules. For example, a functionalized surface may be disposed in a 96 well plate or a 384 well plate. A surface may be disposed on one or more particles. In some embodiments, the one or more particles may be disposed in one or more wells or depressions. A surface may be disposed on a plurality of particles packed in a channel or a porous material disposed in a channel. A surface may be disposed on an inner surface of a channel. A surface may comprise 1, 2, 3, 4 or any number of distinct surface regions. In some embodiments, a surface may be disposed on a particle. In some embodiments, a particle may be a porous particle.

A surface may comprise a wide array of physical properties. A physical property of a surface may include surface charge, hydrophobicity, hydrophilicity, acidity, basicity, surface topography, surface curvature, porosity, shape, and any combination thereof.

A surface functionalization may comprise a polymerizable functional group, a positively or negatively charged functional group, a zwitterionic functional group, an acidic or basic functional group, a polar functional group, or any combination thereof. A surface functionalization may comprise carboxyl groups, hydroxyl groups, thiol groups, cyano groups, nitro groups, ammonium groups, alkyl groups, imidazolium groups, sulfonium groups, pyridinium groups, pyrrolidinium groups, phosphonium groups, aminopropyl groups, amine groups, boronic acid groups, N-succinimidyl ester groups, PEG groups, streptavidin, methyl ether groups, triethoxylpropylaminosilane groups, PCP groups, citrate groups, lipoic acid groups, BPEI groups, or any combination thereof. A surface can be the surface of: micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya tum particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-β-amino-ester particles, poly(amido amine) particles, poly lactic-co-glycolic acid particles, polyanhydride particles, bioreducible polymer particles, and 2-(3-aminopropylamino) ethanol particles, and any combination thereof.

Surfaces can comprise various functionalizations. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group.

A small molecule functionalization may comprise a reactive functional group. Non-limiting examples of the reactive functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a surface can be functionalized to carry a net positive surface charge, a net negative surface charge, an approximately neutral charge. The surface can be a zwitterionic surface.

A surface functionalization may comprise a macromolecular functionalization. A macromolecular functionalization may comprise a biomacromolecule, such as a protein or a polynucleotide (e.g., a 100-mer DNA molecule). A macromolecular functionalization may be comprise a protein, polynucleotide, or polysaccharide, or may be comparable in size to any of the aforementioned classes of species. For example, a macromolecular functionalization may comprise a volume of at least 6, 8, 10, 20, 30, 40, 50, 60, 70, 80, 90, 100, 200, 300, 400, 500, 600, 700, 800, 900, 1000, or 2000 nm³. A macromolecular functionalization may comprise a volume of at most 6, 8, 10, 20, 30, 40, 50, 60, 70, 80, 90, 100, 200, 300, 400, 500, 600, 700, 800, 900, 1000, or 2000 nm³. A macromolecular functionalization may comprise a surface area of at least 15, 30, 50, 80, 100, 200, 300, 400, 500, 600, 700, 800, 900, 1000, or 1500 nm². A macromolecular functionalization may comprise a surface area of at most 15, 30, 50, 80, 100, 200, 300, 400, 500, 600, 700, 800, 900, 1000, or 1500 nm². A macromolecular functionalization may comprise a bait molecule.

Biomolecule corona formation can be a highly dynamic process punctuated by time evolution in composition and physical characteristics (e.g., aggregate charge). Biomolecule corona composition can reflect aggregate biomolecule-biomolecule and biomolecule-sensor element binding affinities, wherein biomolecule binding to a sensor element can be driven not only by its affinity for the sensor element itself, but also by its affinity for other biomolecules adsorbed to the sensor element. In some cases, biomolecule binding to a sensor element can be driven by its interaction strength with other biomolecules bound to the sensor element. Thus, a slight change in sample composition can dramatically change the compositions of biomolecule coronas that form from the sample, and the subset of biomolecules bound to a sensor element can intimately reflect a robust population of biomolecules within a sample.

Additionally, there can be potential differences in kinetic and thermodynamic contributions to biomolecule adsorption. In some cases, a biomolecule with relatively low sensor element binding affinity may have rapid binding kinetics, and thus may initially bind in high quantities but over time be displaced by biomolecules with higher affinities for the sensor element. This can impart high order effects on corona formation kinetics, including unique time-dependent affinities between types of biomolecules and sensor elements.

Biomolecule corona formation can be a highly dynamic process that can be punctuated by time evolution in composition and physical characteristics (e.g., aggregate charge). In some cases, biomolecule corona composition reflects aggregate biomolecule-biomolecule and biomolecule-sensor element binding affinities, wherein biomolecule binding to a sensor element is driven not only by its affinity for the sensor element itself, but also by its affinity for other biomolecules adsorbed to the sensor element. In some cases, biomolecule binding to a sensor element can be driven by its interaction strength with other biomolecules bound to the sensor element. Thus, a slight change in sample composition can dramatically change the compositions of biomolecule coronas that form from the sample, and the subset of biomolecules bound to a sensor element can intimately reflect the full population of biomolecules within a sample.

Further complicating this process can be potential differences in kinetic and thermodynamic contributions to biomolecule adsorption. In some cases, a biomolecule with relatively low sensor element binding affinity may have rapid binding kinetics, and thus may initially bind in high quantities but over time be displaced by biomolecules with higher affinities for the sensor element. This can impart high order effects on corona formation kinetics, including unique time-dependent affinities between types of biomolecules and sensor elements.

Without being limited by theory, aspects of the present disclosure provide methods for assaying a sample using substrates or sensor elements (e.g., nanomaterials such as nanoparticles) which promote crowding or packing of the biomolecules (e.g., proteins) on the sensor element, by at least reducing total capacity by reducing sensor element surface area. In some cases, a higher abundance, but lower affinity biomolecule may be displaced by a lower abundance, but higher affinity biomolecule for a given sensor element. Furthermore, if sensor element surface area is the limiting substrate in the assay, then, the scarcity of sensor element surface and its propensity to reach equilibrium in protein binding can result in preferentially sampling the highest affinity proteins for the sensor element surface or the highest affinity biomolecule-biomolecule interactions, such that the relative abundance of the biomolecule in the sample becomes less critical, and thus, being able to sample more lower abundance biomolecules. In some cases, without being limited by theory, lower sensor element surface area can promote crowding that allows the methods disclosed herein of assaying using nanomaterials to display unique features. For instance, a sensor element, such as nanoparticles disclosed herein, may be designed such as to take advantage of this crowding to compress the proteins on the surface of the particle, promoting some degree of preference for the surface. The use of a sensor element in accordance with the methods disclosure here may also compress the dynamic range of the biomolecules in the sample. The methods disclosed herein can reduce the total amount of protein recovered from a sample and increase the biomolecules (e.g., proteins, protein groups, including unique protein groups that are distinct from one another) detected. This can allow for deep interrogation of a sample, which may not be possible using other methods.

The relationship between the mass input or aggregate surface area of sensor elements (e.g., a particle or nanomaterial surface) and the amount of biomolecules collected on the sensor elements can be complex. In some cases, increasing the mass input or aggregate surface area of sensor elements can increase the total capacity for biomolecule adsorption, thus allowing for a greater mass of biomolecules to be recovered in an assay. However, sensor element aggregate surface area can be inversely proportional to the ratio between the aggregate sensor element surface area and the amount of biomolecules collected on the sensor elements. For example, doubling the number of sensor elements such as particles in a solution of plasma could increase the number of particle adsorbed proteins by a factor of 1.5, coupled with a 25% decrease in the ratio of the number of adsorbed proteins to the aggregate particle surface area.

In some aspects, the compositions and methods disclosed herein provide particles that are capable of capturing low abundance biomolecules from a sample and compressing the dynamic range of biomolecules in a sample upon incubation of said sensor element with said sample. In some aspects, the methods disclosed herein can capture low abundance biomolecules even in low volume samples, where biomolecule capture may be especially difficult.

In some aspects, provided herein are compositions of sensor elements (e.g., surfaces or particles) that may be incubated with various biological samples. In some aspects, the compositions comprise various particle types, alone or in combination, which can be incubated with a wide range of biological samples to analyze the biomolecules (e.g., proteins) present in said biological sample based on binding to particle surface to form protein coronas. A single particle type may be used to assay the proteins in a particular biological sample or multiple particle types can be used together to assay the proteins in the biological sample. A protein corona analysis may be performed on a biological sample (e.g., a biofluid) by contacting the biological sample with a plurality of particles, incubating the biological sample with the plurality of particles to form a protein corona, separating the particles from the biological sample, and analyzing the protein corona to determine the composition of the protein corona. In some embodiments, analyzing the protein corona is performed using mass spectrometry. Interrogation of a sample with a plurality of particles followed by analysis of the protein corona formed on the plurality of particles may be referred to herein as “protein corona analysis.” Abiological sample may be interrogated with one or more particle types. The protein corona of each particle type may be analyzed separately. In some embodiments, the protein corona of one or more particle types may be analyzed in combination.

The present disclosure provides several biological samples that can be assayed using the particles disclosed herein and the methods provided herein. For example, a biological sample may be a biofluid sample such as cerebral spinal fluid (CSF), synovial fluid (SF), urine, plasma, serum, tears, crevicular fluid, semen, whole blood, milk, nipple aspirate, ductal lavage, vaginal fluid, nasal fluid, ear fluid, gastric fluid, pancreatic fluid, trabecular fluid, lung lavage, prostatic fluid, sputum, fecal matter, bronchial lavage, fluid from swabbings, bronchial aspirants, sweat or saliva. A biofluid may be a fluidized solid, for example a tissue homogenate, or a fluid extracted from a biological sample. A biological sample may be, for example, a tissue sample or a fine needle aspiration (FNA) sample. In some embodiments a biological sample may be a cell culture sample. In some embodiments, a biofluid is a fluidized biological sample. For example, a biofluid may be a fluidized cell culture extract.

As used herein, the term “substrate” generally refers to an element that is capable of binding to or adsorbing (e.g., non-specifically) a plurality of biomolecules when in contact with a sample (e.g., a biological sample comprising biomolecules). A substrate may comprise a discrete structure (e.g., a particle) or a portion of a structure (e.g., a surface of a nanomaterial). In one embodiment, the substrate is an element from about 5 nanometers (nm) to about 50000 nm in at least one direction. Suitable substrates include, for example, but not limited to a substrate from about 5 nm to about 50,000 nm in at least one direction, including, about 5 nm to about 40000 nm, alternatively about 5 nm to about 30000 nm, alternatively about 5 nm to about 20,000 nm, alternatively about 5 nm to about 10,000 nm, alternatively about 5 nm to about 5000 nm, alternatively about 5 nm to about 1000 nm, alternatively about 5 nm to about 500 nm, alternatively about 5 nm to 50 nm, alternatively about 10 nm to 100 nm, alternatively about 20 nm to 200 nm, alternatively about 30 nm to 300 nm, alternatively about 40 nm to 400 nm, alternatively about 50 nm to 500 nm, alternatively about 60 nm to 600 nm, alternatively about 70 nm to 700 nm, alternatively about 80 nm to 800 nm, alternatively about 90 nm to 900 nm, alternatively about 100 nm to 1000 nm, alternatively about 1000 nm to 10000 nm, alternatively about 10000 nm to 50000 nm and any combination or amount in between (e.g. 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 125 nm, 150 nm, 175 nm, 200 nm, 225 nm, 250 nm, 275 nm, 300 nm, 350 nm, 400 nm, 450 nm, 500 nm, 550 nm, 600 nm, 650 nm, 700 nm, 750 nm, 800 nm, 850 nm, 900 nm, 1000 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 4500 nm, 5000 nm, 5500 nm, 6000 nm, 6500 nm, 7000 nm, 7500 nm, 8000 nm, 8500 nm, 9000 nm, 10000 nm, 11000 nm, 12000 nm, 13000 nm, 14000 nm, 15000 nm, 16000 nm, 17000 nm, 18000 nm, 19000 nm, 20000 nm, 25000 nm, 30000 nm, 35000 nm, 40000 nm, 45000 nm, 50000 nm and any number in between). The substrate may comprise a “nanoscale substrate.” A nanoscale substrate generally refers to a substrate that is less than 1 micron in at least one direction. Suitable examples of ranges of nanoscale substrates include, but are not limited to, for example, elements from about 5 nm to about 1000 nm in one direction, including, from example, about 5 nm to about 500 nm, alternatively about 5 nm to about 400 nm, alternatively about 5 nm to about 300 nm, alternatively about 5 nm to about 200 nm, alternatively about 5 nm to about 100 nm, alternatively about 5 nm to about 50 nm, alternatively about 10 nm to about 1000 nm, alternatively about 10 nm to about 750 nm, alternatively about 10 nm to about 500 nm, alternatively about 10 nm to about 250 nm, alternatively about 10 nm to about 200 nm, alternatively about 10 nm to about 100 nm, alternatively about 50 nm to about 1000 nm, alternatively about 50 nm to about 500 nm, alternatively about 50 nm to about 250 nm, alternatively about 50 nm to about 200 nm, alternatively about 50 nm to about 100 nm, and any combinations, ranges or amount in-between (e.g. 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 125 nm, 150 nm, 175 nm, 200 nm, 225 nm, 250 nm, 275 nm, 300 nm, 350 nm, 400 nm, 450 nm, 500 nm, 550 nm, 600 nm, 650 nm, 700 nm, 750 nm, 800 nm, 850 nm, 900 nm, 1000 nm, etc.). In reference to the sensor arrays described herein, the use of the term substrate includes the use of a nanoscale substrate for the sensor and associated methods.

The term “biomolecule corona” generally refers to a composition, signature or pattern of different biomolecules or biomolecule groups associated with (e.g., bound to, adsorbed to) each separate substrate or a portion thereof (e.g., a surface of a substrate). The biomolecule corona not only refers to the different biomolecules but also the differences in the amount, level or quantity of the biomolecule bound to the substrate, or differences in the conformational state of the biomolecule that is bound to the substrate. In some cases, biomolecule coronas corresponding to different substrates may comprise common biomolecules, may contain distinct biomolecules with regard to the other substrates, and/or may differ in level or quantity, type or confirmation of the biomolecule. The biomolecule corona may depend on not only the physicochemical properties of the substrate, but also the nature of the sample, the duration of exposure, and/or a concentration of the substrate.

A biomolecule corona may comprise proteins, saccharides, lipids, metabolites, nucleic acids, or any combination thereof. In some cases, the biomolecule corona is a protein corona. In another case, the biomolecule corona is a polysaccharide corona. In yet another case, the biomolecule corona is a metabolite corona. In some cases, the biomolecule corona is a lipidomic corona.

Biomolecule corona composition is often a complex function of condition dependent on intermolecular (e.g., biomolecule-biomolecule), substrate, and solvation affinities for all analytes present in a sample. For each analyte in a sample, substrate (e.g., particle) binding can depend not only on solution conditions, but also on a range of biomolecule-biomolecule interactions on the substrate and in solution. Accordingly, the complexity of biomolecule corona data can be prohibitive for certain forms of quantitative sample analysis, such as absolute abundance determinations.

In spite of this underlying complexity, many biomolecules exhibit strong dependencies on substrate (e.g., particle) concentration, surface area, and mass. The relationship between substrate quantity and biomolecule corona composition can provide quantitative handles for quantitatively analyzing biological samples. Further disclosed herein are methods for exploiting substrate concentration trends for enhanced biological profiling depth, dynamic range, and accuracy (e.g., diminished inter-replicate variability).

### Particle Types

Particle types consistent with the methods disclosed herein can be made from various materials. For example, particle materials consistent with the present disclosure include metals, polymers, magnetic materials, and lipids. Magnetic particles may be iron oxide particles. Examples of metal materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron and cadmium, or any other material described in U.S. Pat. No. 7,749,299. In some embodiments, a particle may be a superparamagnetic iron oxide nanoparticle (SPION). A magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material). For example, a particle core may comprise superparamagnetic γ-ferric iron oxide. A particle may comprise a distinct core (e.g., the innermost portion of the particle), shell (e.g., the outermost layer of the particle), and shell or shells (e.g., portions of the particle disposed between the core and the shell). In some cases, a core comprises a metal, an oxide, a nitride, a ceramic, a carbon material, a silicon material, a polymer, or any combination thereof. In some cases, a shell comprises a polymer, a saccharide, a lipid, a peptide, a self-assembled monolayer, a sol-gel, a hydrogel, a glass, or any combination thereof. In some cases, a shell comprises polystyrene, N-(3-(Dimethylamino) propyl) methacrylamide (DMAPMA), or a combination thereof. In some cases, a shell material comprises a small molecule functionalization. In some cases, a shell material comprises a biomolecular functionalization (e.g., a peptide or saccharide functional appendage). A particle may comprise a uniform composition. A core or a shell may comprise a plurality of materials comprising a degree of phase separation. For example, a shell may comprise two phase separated polymers. A particle core and shell may comprise different densities. A shell material may comprise a thickness of at least 2 nm, at least 4 nm, at least 5 nm, at least 8 nm, at least 10 nm, at least 15 nm, at least 20 nm, at least 25 nm, at least 30 nm, or at least 35 nm. A shell material may comprise a thickness of at most 35 nm, at most 30 nm, at most 25 nm, at most 20 nm, at most 15 nm, at most 10 nm, at most 8 nm, at most 5 nm, at most 4 nm, or at most 2 nm.

A particle may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). In some embodiments, the polymer is a lipid-terminated polyalkylene glycol and a polyester, or any other material disclosed in U.S. Pat. No. 9,549,901.

A particle may comprise a lipid. A lipid-containing particle may comprise a lipid coupled to its surface (e.g., covalently attached to a surface amine of the particle or non-covalently bound by a particle-bound lipid binding protein), or may comprise a lipid within a monolayer or bilayer comprising the lipid. A lipid monolayer or bilayer may comprise non-lipidic biomolecules, including sterols, proteins (e.g., clathrins), and saccharides. A plurality of lipids associated with a particle may be fully or partially polymerized. A particle may comprise a liposome. Examples of lipids that can be used to form the particles of the present disclosure include cationic, anionic, and neutrally charged lipids. For example, particles can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol, or any other material listed in U.S. Pat. No. 9,445,994, which is incorporated herein by reference in its entirety.

Examples of particles of the present disclosure are provided in TABLE 1.

TABLE 1
Example particles of the present disclosure

| Batch No. | Description |
|---|---|
| NP-F | Carboxylate (Citrate) superparamagnetic iron oxide NPs (SPION) |
| NP-M | Phenol-formaldehyde coated SPION |
| NP-C | Silica-coated superparamagnetic iron oxide NPs (SPION) |
| NP-N | Polystyrene coated SPION |
| NP-G | Carboxylated Poly(styrene-co-methacrylic acid), P(St-co-MAA) |
| | coated SPION |
| NP-D | N-(3-Trimethoxysilylpropyl)diethylenetriamine coated SPION |
| NP-E | poly(N-(3-(dimethylamino)propyl) methacrylamide) |
| | (PDMAPMA)-coated SPION |
| NP-O | 1,2,4,5-Benzenetetracarboxylic acid coated SPION |
| NP-H | poly(vinylbenzyltrimethylammonium chloride) (PVBTMAC) |
| | coated SPION |
| NP-P | Carboxylate, PAA coated SPION |
| NP-Q | poly(oligo(ethylene glycol) methyl ether methacrylate) |
| | (POEGMA)-coated SPION |
| NP-R | Carboxylate microparticle, surfactant free |
| NP-A | Polystyrene carboxyl functionalized |
| NP-S | Carboxylic acid |
| NP-T | Silica |
| NP-U | Carboxylic acid, 150 nm |
| NP-V | Amino surface microparticle, 0.4-0.6 μm |
| NP-W | Silica amino functionalized microparticle, 0.1-0.39 μm |
| NP-X | Jeffamine surface, 0.1-0.39 μm |
| NP-Y | Polystyrene microparticle, 2.0-2.9 μm |
| NP-Z | Silica |
| NP-AA | Carboxylated Original coating, 50 nm |
| NP-B | Dextran based coating, 0.13 μm |
| NP-I | Silica Silanol coated with lower acidity |
| NP-AB | 1,6-hexanediamine functionalized SPION |
| NP-AC | Mixed amide, carboxylate functionalized, silica-coated SPION |
| NP-AD | N¹-(3-(trimethoxysilyl)propyl)hexane-1,6-diamine functionalized, |
| | silica-coated SPION |

A particle of the present disclosure may be synthesized, or a particle of the present disclosure may be purchased from a commercial vendor. For example, particles consistent with the present disclosure may be purchased from commercial vendors including Sigma-Aldrich, Life Technologies, Fisher Biosciences, nanoComposix, Nanopartz, Spherotech, and other commercial vendors. In some embodiments, a particle of the present disclosure may be purchased from a commercial vendor and further modified, coated, or functionalized.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl) diethylenetriamine coated SPION, a poly(N-(3-(dimethylamino) propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo (ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity.

Particles that are consistent with the present disclosure can be made and used in methods of forming protein coronas after incubation in a biofluid at a wide range of sizes. In some cases, a particle of the present disclosure may be a nanoparticle. In some cases, a nanoparticle of the present disclosure may be from about 10 nm to about 1000 nm in diameter. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. In some cases, a nanoparticle may be less than 1000 nm in diameter. In some cases, a particle comprises a diameter of about 30 nm to about 800 nm. In some cases, a particle comprises a diameter of about 60 nm to about 600 nm. In some cases, a particle comprises a diameter of about 60 nm to about 500 nm. In some cases, a particle comprises a diameter of about 60 nm to about 400 nm. In some cases, a particle comprises a diameter of about 60 nm to about 300 nm. In some cases, a particle comprises a diameter of about 60 nm to about 200 nm. In some cases, a particle comprises a diameter of about 60 nm to about 150 nm. In some cases, a particle comprises a diameter of about 80 nm to about 500 nm. In some cases, a particle comprises a diameter of about 80 nm to about 400 nm. In some cases, a particle comprises a diameter of about 80 nm to about 300 nm. In some cases, a particle comprises a diameter of about 80 nm to about 200 nm. In some cases, a particle comprises a diameter of about 80 nm to about 150 nm. In some cases, a particle comprises a diameter of about 100 nm to about 500 nm. In some cases, a particle comprises a diameter of about 100 nm to about 400 nm. In some cases, a particle comprises a diameter of about 100 nm to about 300 nm. In some cases, a particle comprises a diameter of about 100 nm to about 200 nm. In some cases, a particle comprises a diameter of about 100 nm to about 150 nm. In some cases, a particle comprises a diameter of about 120 nm to about 600 nm. In some cases, a particle comprises a diameter of about 120 nm to about 500 nm. In some cases, a particle comprises a diameter of about 120 nm to about 400 nm. In some cases, a particle comprises a diameter of about 120 nm to about 350 nm. In some cases, a particle comprises a diameter of about 120 nm to about 300 nm. In some cases, a particle comprises a diameter of about 120 nm to about 200 nm. In some cases, a particle comprises a diameter of about 150 nm to about 600 nm. In some cases, a particle comprises a diameter of about 150 nm to about 500 nm. In some cases, a particle comprises a diameter of about 150 nm to about 400 nm. In some cases, a particle comprises a diameter of about 150 nm to about 300 nm. In some cases, a particle comprises a diameter of about 200 nm to about 400 nm. In some cases, a particle comprises a diameter of about 200 nm to about 600 nm. In some cases, a particle comprises a diameter of at least about 100 nm. In some cases, a particle comprises a diameter of at most 500 nm.

In some cases, a particle of the present disclosure may be a microparticle. A microparticle may be a particle that is from about 1 μm to about 1000 μm in diameter. For example, the microparticles disclosed here can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. In some cases, a microparticle may be less than 1000 μm in diameter. In some cases, a microparticle comprises a diameter of about 1 μm to about 2 μm. In some cases, a microparticle comprises a diameter of about 1 μm to about 1.5 μm.

A substrate (such as a particle) may comprise a degree of shape or size uniformity or non-uniformity. A physical measure of such heterogeneity may be polydispersity, which tracks size uniformity of a substrate, and may be defined as the square of the ratio of the standard deviation and the mean of substrate size (e.g., particle diameter). Alternatively, polydispersity may be a ratio of (1) weight average molecular weight to (2) number average molecular weight for a substrate (e.g., for a collection of particles), and therefore serves as a measure of mass variance for the substrate. A substrate may comprise a low polydispersity value, indicating a high degree of size uniformity. For example, a substrate (e.g., a collection of a substrate comprising a plurality of copies of the substrate) may comprise a polydispersity index of at most 1.6, at most 1.4, at most 1.2, at most 1, at most 0.8, at most 0.6, at most 0.5, at most 0.4, at most 0.3, at most 0.25, at most 0.2, at most 0.15, at most 0.1, at most 0.05, at most 0.03, or at most 0.02. Alternatively, a substrate may comprise a high polydispersity index, indicating a degree of size and/or mass variation. For example, a substrate (e.g., a collection of a substrate comprising a plurality of copies of the substrate) may comprise a polydispersity index of at least 0.3, at least 0.4, at least 0.5, at least 0.6, at least 0.8, at least 1, at least 1.2, at least 1.4, at least 1.6, at least 1.8, at least 2, at least 2.2, at least 2.5, or at least 3.

A particle may be substantially spherical. A particle may comprise an oblong geometry. A particle may comprise a surface feature, such as a well, a trench, or a substantially flat region.

A particle may be provided at a range of concentrations. A particle may comprise a concentration of at least 10 pM. A particle may comprise a concentration of at least 100 pM. A particle may comprise a concentration of at least 1 nM. A particle may comprise a concentration of at least 10 nM. A particle may comprise a concentration of at most 100 nM. A particle may comprise a concentration of at most 10 nM. A particle may comprise a concentration of at most 1 nM. A particle may comprise a concentration of at most 100 pM. A particle may comprise a concentration of at most 10 pM. A particle may comprise a concentration of at most 1 pM. A particle may comprise a concentration between 100 fM and 100 nM. A particle may comprise a concentration between 100 fM and 10 pM. A particle may comprise a concentration between 1 pM and 100 pM. A particle may comprise a concentration between 10 pM and 1 nM. A particle may comprise a concentration between 100 pM and 10 nM. A particle may comprise a concentration between 1 nM and 100 nM. A particle may comprise a concentration of at least 10 ng/ml. A particle may comprise a concentration of at least 100 ng/ml. A particle may comprise a concentration of at least 1 μg/ml. A particle may comprise a concentration of at least 10 μg/ml. A particle may comprise a concentration of at least 100 μg/ml. A particle may comprise a concentration of at least 1 mg/ml. A particle may comprise a concentration of at least mg/ml. A particle may comprise a concentration of at least 10 mg/ml. A particle may comprise a concentration of at most 10 mg/ml. A particle may comprise a concentration of at most 1/ml. A particle may comprise a concentration of at most 100 μg/ml. A particle may comprise a concentration of at most 10 μg/ml. A particle may comprise a concentration of at most 1 μg/ml. A particle may comprise a concentration of at most 100 ng/ml. A particle may comprise a concentration of at most 10 ng/ml.

A particle may be contacted to a biological sample at a range of volume ratios. A solution comprising a particle may be combined with a biological sample, at a volume ratio of greater than about 100:1, about 100:1, about 80:1, about 60:1, about 50:1, about 40:1, about 30:1, about 25:1, about 20:1, about 15:1, about 12:1, about 10:1, about 8:1, about 6:1, about 5:1, about 4:1, about 3:1, about 5:2, about 2:1, about 3:2, about 1:1, about 2:3, about 1:2, about 2:5, about 1:3, about 1:4, about 1:5, about 1:6, about 1:8, about 1:10, about 1:12, about 1:15, about 1:20, about 1:25, about 1:30, about 1:40, about 1:50, about 1:60, about 1:80, about 1:100, or less than about 1:100.

The ratio between surface area and mass can be a determinant of a particle's properties. For example, the number and types of biomolecules that a particle adsorbs from a solution may vary with the particle's surface area to mass ratio. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg.

In some cases, a plurality of particles (e.g., of a particle panel) used with the methods described herein may have a range of surface area to mass ratios. In some cases, the range of surface area to mass ratios for a plurality of particles is less than 100 cm²/mg, 80 cm²/mg, 60 cm²/mg, 40 cm²/mg, 20 cm²/mg, 10 cm²/mg, 5 cm²/mg, or 2 cm²/mg. In some cases, the surface area to mass ratios for a plurality of particles varies by no more than 40%, 30%, 20%, 10%, 5%, 3%, 2%, or 1% between the particles in the plurality. In some cases, the plurality of particles may comprise at least 2, 3, 4, 5, 6, 7, 8, 9, 10, 12, 15, 20, or more different types of particles.

In some cases, a plurality of particles (e.g., in a particle panel) may comprise a range of surface area to mass ratios. In some cases, the range of surface area to mass ratios for a plurality of particles is greater than 100 cm²/mg, 150 cm²/mg, 200 cm²/mg, 250 cm²/mg, 300 cm²/mg, 400 cm²/mg, 500 cm²/mg, 800 cm²/mg, 1000 cm²/mg, 1200 cm²/mg, 1500 cm²/mg, 2000 cm²/mg, 3000 cm²/mg, 5000 cm²/mg, 6000 cm²/mg, 7500 cm²/mg, 10000 cm²/mg, or more. In some cases, the surface area to mass ratios for a plurality of particles (e.g., within a panel) can vary by more than 100%, 200%, 300%, 400%, 500%, 1000%, 10000% or more. In some cases, the plurality of particles with a wide range of surface area to mass ratios comprises at least 2, 3, 4, 5, 6, 7, 8, 9, 10, 12, 15, 20, or more different types of particles.

A particle may comprise a wide range of physical properties. A physical property of a particle may include composition, size, surface charge, hydrophobicity, hydrophilicity, surface functionalization, surface topography, surface curvature, porosity, core material, shell material, shape, and any combination thereof.

A surface functionalization may comprise a polymerizable functional group, a positively or negatively charged functional group, a zwitterionic functional group, an acidic or basic functional group, a polar functional group, or any combination thereof. In some cases, a surface functionalization comprises a polar functional group, an acidic functional group, a basic functional group, a charged functional group, a polymerizable functional group, or any combination thereof. In some cases, a surface functionalization comprises an aminopropyl functionalization, an amine functionalization, a boronic acid functionalization, a carboxylic acid functionalization, a methyl functionalization, an N-succinimidyl ester functionalization, a PEG functionalization, a streptavidin functionalization, a methyl ether functionalization, a triethoxylpropylaminosilane functionalization, a thiol functionalization, a PCP functionalization, a citrate functionalization, a lipoic acid functionalization, a BPEI functionalization, carboxyl functionalization, a hydroxyl functionalization, or any combination thereof. A surface functionalization may comprise carboxyl groups, hydroxyl groups, thiol groups, cyano groups, nitro groups, ammonium groups, alkyl groups, imidazolium groups, sulfonium groups, pyridinium groups, pyrrolidinium groups, phosphonium groups, aminopropyl groups, amine groups, boronic acid groups, N-succinimidyl ester groups, PEG groups, streptavidin, methyl ether groups, triethoxylpropylaminosilane groups, PCP groups, citrate groups, lipoic acid groups, BPEI groups, or any combination thereof. A surface functionalization may be present at a range of densities on a particle. In some cases, a surface functionalization comprises an average density of at least about 1 functional group per 20 nm² on a surface of a particle. In some cases, a surface functionalization comprises an average density of at least about 1 functional group per 30 nm² on a surface of a particle. In some cases, a surface functionalization comprises an average density of at least about 1 functional group per 40 nm² on a surface of a particle. In some cases, a surface functionalization comprises an average density of at least about 1 functional group per 50 nm² on a surface of a particle. In some cases, a surface functionalization comprises an average density of at least about 1 functional group per 60 nm² on a surface of a particle. In some cases, a surface functionalization comprises an average density of at least about 1 functional group per 80 nm² on a surface of a particle. In some cases, a surface functionalization comprises an average density of at most about 1 functional group per 80 nm² on a surface of a particle. In some cases, a surface functionalization comprises an average density of at most about 1 functional group per 60 nm² on a surface of a particle. In some cases, a surface functionalization comprises an average density of at most about 1 functional group per 50 nm² on a surface of a particle. In some cases, a surface functionalization comprises an average density of at most about 1 functional group per 40 nm² on a surface of a particle. In some cases, a surface functionalization comprises an average density of at most about 1 functional group per 30 nm² on a surface of a particle. In some cases, a surface functionalization comprises an average density of at most about 1 functional group per 20 nm² on a surface of a particle. In some cases, a surface functionalization comprises an average density about 1 functional group per 20 nm² to at most about 1 functional group per 60 nm² on a surface of a particle.

A particle may be selected from the group consisting of: micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya tum particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-β-amino-ester particles, poly(amido amine) particles, poly lactic-co-glycolic acid particles, polyanhydride particles, bioreducible polymer particles, and 2-(3-aminopropylamino) ethanol particles, and any combination thereof.

Particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group.

A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. A zwitterionic particle surface may be zwitterionic over at least 1, at least 2, at least 3, at least 4, at least 5, at least 6 or more pH units. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

### Particle Panels

The present disclosure provides compositions and methods of use thereof for assaying a sample for proteins. Compositions described herein include particle panels comprising one or more than one distinct particle types. Particle panels described herein can vary in the number of particle types and the diversity of particle types in a single panel. For example, particles in a panel may vary based on size, polydispersity, shape and morphology, surface charge, surface chemistry and functionalization, and base material. Panels may be incubated with a sample to be analyzed for protein composition. Proteins in the sample adsorb to the surface of the different particle types in the particle panel to form a protein corona. The types of proteins which adsorb to a certain particle type in the particle panel may depend on the composition, size, and surface charge of said particle type. Thus, each particle type in a panel may have different protein coronas due to adsorbing a different set of proteins, different concentrations of a particular protein, or a combination thereof. Each particle type in a panel may have mutually exclusive protein coronas or may have overlapping protein coronas. Overlapping protein coronas can overlap in protein identity, in protein concentration, or both.

The present disclosure also provides methods for selecting a particle type for inclusion in a panel depending on the sample type. Particle types included in a panel may be a combination of particles that are optimized for removal of highly abundant proteins. Particle types also consistent for inclusion in a panel are those selected for adsorbing particular proteins of interest. The particles can be nanoparticles. The particles can be microparticles. The particles can be a combination of nanoparticles and microparticles.

A particle panel including any number of distinct particle types disclosed herein, enriches and identifies a single protein or protein group. In some cases, the single protein or protein group may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the particle panel including any number of distinct particle types disclosed herein, enriches and identifies a single protein or protein group by binding different domains, sequences, or epitopes of the single protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group.

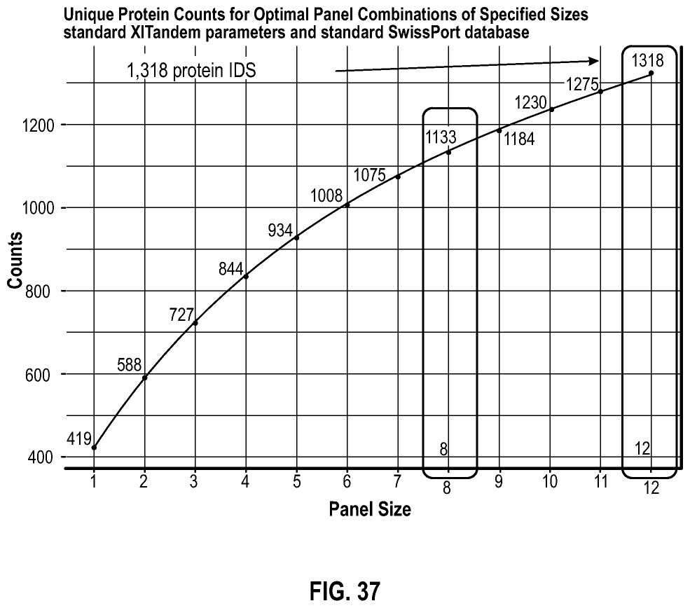

A particle panel can have more than one particle type. Increasing the number of particle types in a panel can be a method for increasing the number of proteins that can be identified in a given sample. An example of how increasing panel size may increase the number of identified proteins is shown in FIG. 37, in which a panel size of one particle type identified 419 different proteins, a panel size of two particle types identified 588 different proteins, a panel size of three particle types identified 727 different proteins, a panel size of four particle types identified 844 proteins, a panel size of five particle types identified 934 different proteins, a panel size of six particle types identified 1008 different proteins, a panel size of seven particle types identified 1075 different proteins, a panel size of eight particle types identified 1133 different proteins, a panel size of nine particle types identified 1184 different proteins, a panel size of 10 particle types identified 1230 different proteins, a panel size of 11 particle types identified 1275 different proteins, and a panel size of 12 particle types identified 1318 different proteins.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino) propyl]methacrylamide-co-[2-(methacryloyloxy)ethyl]dimethyl-(3-sulfopropyl) ammonium hydroxide, P (DMAPMA-co-SBMA) coated particle. A particle panel consistent with the present disclosure may comprise silica-coated particles, N-(3-Trimethoxysilylpropyl) diethylenetriamine coated particles, poly(N-(3-(dimethylamino) propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl) diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

A particle panel consistent with the present disclosure may comprise a silica particle, an amine functionalized particle, and a polyethylene glycol-functionalized particle. The particle panel may further comprise a carboxylate functionalized particle, such as a carboxylate functionalized styrene particle. The particle panel may further comprise a saccharide-coated particle. In some cases, the saccharide-coated particle is a dextran-coated particle. The particle panel may further comprise a sulfuryl functionalized particle. The sulfuryl functionalized particle may comprise a positively charged surface functionalization such as an amine, and thereby may be zwitterionic. The particle panel may further comprise a particle with a boronated or boronic acid functionalized surface. The particle panel may further comprise a particle with an oleic acid functionalized surface. The particle panel may comprise at least one microparticle.

The present disclosure includes compositions (e.g., particle panels) and methods that comprise two or more particles differing in at least one physicochemical property. A composition or method of the present disclosure may comprise 3 to 6 particles differing in at least one physicochemical property. A composition or method of the present disclosure may comprise 4 to 8 particles differing in at least one physicochemical property. A composition or method of the present disclosure may comprise 4 to 10 particles differing in at least one physicochemical property. A composition or method of the present disclosure may comprise 5 to 12 particles differing in at least one physicochemical property. A composition or method of the present disclosure may comprise 6 to 14 particles differing in at least one physicochemical property. A composition or method of the present disclosure may comprise 8 to 15 particles differing in at least one physicochemical property. A composition or method of the present disclosure may comprise 10 to 20 particles differing in at least one physicochemical property. A composition or method of the present disclosure may comprise at least 2 distinct particle types, at least 3 distinct particle types, at least 4 distinct particle types, at least 5 distinct particle types, at least 6 distinct particle types, at least 7 distinct particle types, at least 8 distinct particle types, at least 9 distinct particle types, at least 10 distinct particle types, at least 11 distinct particle types, at least 12 distinct particle types, at least 13 distinct particle types, at least 14 distinct particle types, at least 15 distinct particle types, at least 20 distinct particle types, at least 25 particle types, or at least 30 distinct particle types.

A particle panel of the present disclosure may comprise at least one, at least two, at least 3, at least 4, or each particle selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. A particle panel of the present disclosure may comprise a SPION comprising a poly(N-(3-(dimethylamino) propyl) methacrylamide) (PDMAPMA) surface. A particle panel of the present disclosure may comprise a SPION comprising a poly(oligo (ethylene glycol) methyl ether methacrylate) (POEGMA) surface. A particle panel of the present disclosure may comprise a SPION comprising an N-(3-Trimethoxysilylpropyl) diethylenetriamine surface. A particle panel of the present disclosure may comprise a SPION comprising a Poly(dimethyl aminopropyl methacrylamide) (Dimethylamine) surface. A particle panel of the present disclosure may comprise a SPION comprising a dextran surface. A particle panel of the present disclosure may comprise a SPION comprising a surface with a mixed chemistry based on amine-epoxy chemistry. A particle panel of the present disclosure may comprise a SPION comprising a Polyzwitterion coated (Poly(N-[3-(Dimethylamino) propyl]methacrylamide-co-[2-(methacryloyloxy)ethyl]dimethyl-(3-sulfopropyl) ammonium hydroxide, P (DMAPMA-co-SBMA)) surface. A particle panel of the present disclosure may comprise a SPION comprising styrene surface comprising an oleic acid functionalization. A particle panel of the present disclosure may comprise a SPION comprising a boronated styrene surface. A particle panel of the present disclosure may comprise a SPION comprising a carboxylated styrene surface. A particle panel of the present disclosure may comprise a SPION comprising a carboxylated styrene surface. A particle panel of the present disclosure may comprise a SPION comprising a strongly acidic silica surface. A particle panel of the present disclosure may comprise at least one particle, at least 2 particles, at least 3 particles, or at least 4 particles selected from the group consisting of a silica-coated SPION, a poly(dimethylaminopropylmethacrylamide)-coated SPION, an N-(3-Trimethoxysilylpropyl) diethylenetriamine-coated SPION, a 1,6-hexanediamine-coated SPION, and an N¹-(3-(trimethoxysilyl) propyl) hexane-1,6-diamine functionalized, silica-coated SPION. A particle panel of the present disclosure may comprise a silica-coated SPION, a poly(dimethylaminopropylmethacrylamide)-coated SPION, an N-(3-Trimethoxysilylpropyl) diethylenetriamine-coated SPION, a 1,6-hexanediamine-coated SPION, and an N¹-(3-(trimethoxysilyl) propyl) hexane-1,6-diamine functionalized, silica-coated SPION.

### Biomolecule Coronas

The present disclosure provides a variety of compositions, systems, and methods for collecting biomolecules on nanoparticles and microparticles (as well as other types of sensor elements such as polymer matrices, filters, rods, and extended surfaces). A particle may adsorb a plurality of biomolecules upon contact with a biological sample, thereby forming a biomolecule corona on its surface. The biomolecule corona may comprise proteins, lipids, nucleic acids, metabolites, saccharides, small molecules (e.g., sterols), and other biological species present in a sample. A biomolecule corona comprising proteins may also be referred to as a ‘protein corona’, and may refer to all constituents adsorbed to a particle (e.g., proteins, lipids, nucleic acids, and other biomolecules), or may refer only to proteins adsorbed to the particle.

A particle of the present disclosure may be contacted with a biological sample (e.g., a biofluid) to form a biomolecule corona. The particle and biomolecule corona may be separated from the biological sample, for example by centrifugation, ultracentrifugation, density or gradient-based centrifugation, magnetic separation, filtration, chromatographic separation, gravitational separation, charge-based separation, column-based separation, spin column-based separation, or any combination thereof. In some cases, the particle is magnetically separated from the sample. Each of a plurality of particle types may be separated from a biological sample or from a mixture of particles based on their physical, chemical, charge, or magnetic properties. Protein corona analysis may also be performed on the separated particle and biomolecule corona. Protein corona analysis may comprise identifying one or more proteins in the biomolecule corona, for example by mass spectrometry. A single particle type (e.g., a particle of a type listed in TABLE 1) may be contacted to a biological sample. A plurality of particle types (e.g., a plurality of the particle types provided in TABLE 1) may be contacted to a biological sample. The plurality of particle types may be combined and contacted to the biological sample in a single sample volume. The plurality of particle types may be sequentially contacted to a biological sample and separated from the biological sample prior to contacting a subsequent particle type to the biological sample. Protein corona analysis of the biomolecule corona may compress the dynamic range of the analysis compared to a total protein analysis method.

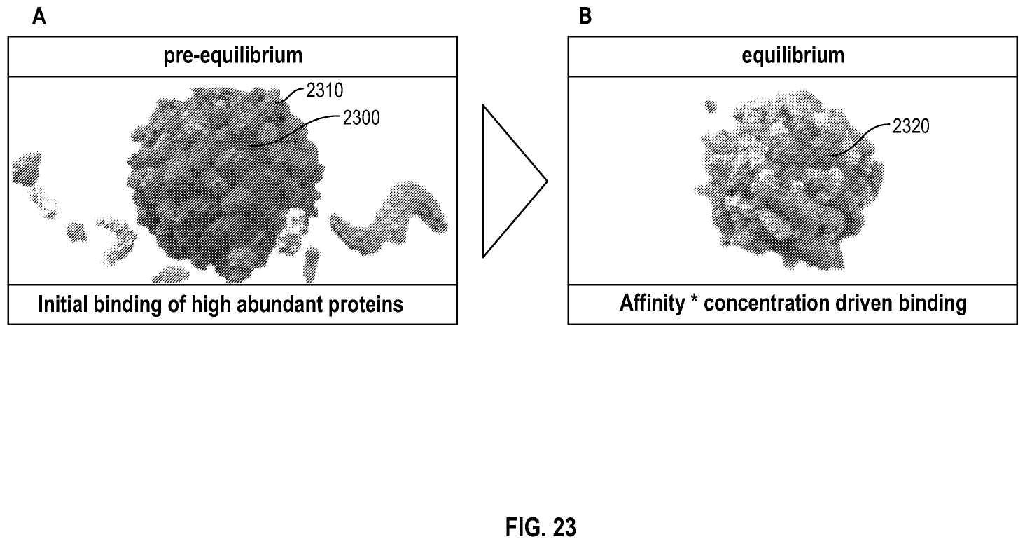

Biomolecule corona formation may comprise a time dependence, such that biomolecule corona size, charge, and composition may change over time. This concept is illustrated in FIG. 23, with FIG. 23 panel A depicting a particle 2300 transiently bound to fast-binding proteins 2310 at an early timepoint during biomolecule corona formation, and FIG. 23 panel B depicting the particle 2300 at a later timepoint, in which the fast-binding proteins 2310 have been replaced by slower-binding proteins 2320 in the biomolecule corona of the particle. Depending on a range of factors including particle physicochemical properties, sample complexity, solution conditions (e.g., temperature and osmolarity), and particle concentration, biomolecule corona composition may not only exhibit time evolution, but may ultimately reach a stable or unstable equilibrium. In many systems, biomolecule corona complexity increases with time. In such cases, a first set of biomolecules which rapidly bind to a substrate (such as a particle) may undergo exchange with solution phase biomolecules, resulting in biomolecule replacement. For many particles, contact with plasma leads to rapid albumin adsorption, followed by gradual albumin substitution by lower abundance proteins.

Particle concentration can be a central determinant for biomolecule corona evolution. Adjusting particle concentration may result in a change in the composition and evolutionary time course of a biomolecule corona. Particle concentration may also affect the rate at which a biomolecule corona approaches equilibrium. Accordingly, in some cases, dynamic range, profiling depth, low abundance biomolecule (e.g., present at less than 10 μg/ml) collection, biomolecule corona diversity, or any combination of traits thereof may be enhanced by lowering particle concentration (e.g., via serial dilution of a particle solution or suspension). The ratio of particle mass or surface area to biomolecule concentration may provide a handle for controlling biomolecule corona composition and formation.

A method of the present disclosure may comprise assaying a sample with multiple concentrations of a particle. For example, a method of the present disclosure may comprise contacting a first portion of a biological sample with a first concentration of a particle, thereby generating a first biomolecule corona; contacting a second portion of the biological sample with a second concentration of the particle, thereby generating a second biomolecule corona, and assaying the first biomolecule corona and the second biomolecule corona to identify biomolecules or biomolecule groups comprised therein. In some cases, the assaying generates at least 5%, at least 10%, at least 15%, at least 20%, at least 25%, or at least 30% greater average number of signals per identified biomolecule than assaying either said first biomolecule corona or said second biomolecule corona alone. In some cases, the assaying comprises identifying at least 1, at least 2, at least 5, at least 10, at least 20, at least 30, at least 50, at least 80, at least 100, at least 150, at least 200, or at least 250 biomolecules or a biomolecule groups which are not identifiable from assaying said first biomolecule corona or said second biomolecule corona alone. In some cases, a dynamic range of the identified biomolecules or biomolecule groups is at least 0.5, at least 1, at least 1.5, or at least 2 greater than dynamic ranges of the biomolecules or biomolecule groups in both the first biomolecule corona and the second biomolecule corona.

The first concentration and second concentration of the particle may be between 100 nanogram/milliliter (ng/mL) and 100 milligram/milliliter (mg/mL). The first concentration and second concentration of the particle may be between 1 microgram/milliliter (μg/mL) and 50 milligram/milliliter (mg/mL). The first concentration and second concentration of the particle may be between 10 microgram/milliliter (μg/mL) and 20 milligram/milliliter (mg/mL). The first concentration and second concentration of the particle may be between 100 microgram/milliliter (μg/mL) and 10 milligram/milliliter (mg/mL).

The method may be multiplexed to include any number of particle concentrations. For example, the method may be performed by adding portions of the biological sample to a well plate with a plurality of wells comprising a plurality of different concentrations of the particle. Each instance of contacting a portion of the biological sample with a concentration of the particle may comprise identical conditions (e.g., time, pH, temperature), or two or more instances of contacting portions of the biological sample with concentrations of the particle may comprise different conditions. In some cases, the particle contacted to the first portion of the biological sample and the particle contacted toe the second portion of the biological sample comprise substantially similar zeta potentials following formation of the first and second biomolecule coronas.

The particle may comprise a plurality of particles. Particles of the plurality of particles may differ from one another by at least one physicochemical property. In some cases, the physicochemical property comprises surface area to mass ratio. In some cases, the physicochemical property comprises charge. For example, a first particle from the plurality of particles may comprise a positive charge, and a second particle from the plurality of particles may comprise an approximately neutral charge.

Performing an assay with multiple concentrations of particles can provide a handle for identifying low abundance biomolecules from a sample. Low abundance biomolecule collection can be challenged by high abundance biomolecules (e.g., albumin in plasma), which can competitively low concentration biomolecule particle adsorption through competitive binding. In some cases, a first concentration of a particle and a second concentration of a particle generate biomolecule coronas with different subsets of low abundance biomolecules from the sample. Furthermore, an assay utilizing multiple particle concentrations may generate a biomolecule corona with a relatively low prevalence of high abundance biomolecules. In some cases, a first concentration of a particle and a second concentration of a particle generate biomolecule coronas with different proportions of high abundance biomolecules. In some cases, the ratio of albumin to non-albumin biomolecules in the first biomolecule corona and the second biomolecule corona differ by at least 5%, at least 10%, at least 15%, at least 20%, or at least 25%. In some cases, the ratio of sub-microgram per milliliter biomolecules from the biological sample in the first biomolecule corona and the second biomolecule corona differs by at least 5%, at least 10%, at least 15%, at least 20%, or at least 25%.

The assaying may comprise identifying a thermodynamic parameter for binding of a biomolecule or biomolecule group from said first biomolecule corona or said second biomolecule corona. For example, the assaying may identify a binding enthalpy, binding entropy, binding free energy, binding rate, or equilibrium constant for binding for a biomolecule or biomolecule group from a biomolecule corona.

A particle may be contacted to a biological sample at a range of mass ratios. A sample may comprise at most 1 mg of a particle per 100,000 mg of biomolecules. A sample may comprise at most 1 mg of a particle per 10000 mg of biomolecules. A sample may comprise at most 1 mg of a particle per 1000 mg of biomolecules. A sample may comprise at most 1 mg of a particle per 100 mg of biomolecules. A sample may comprise at most 1 mg of a particle per 10 mg of biomolecules. A sample may comprise at most 1 mg of a particle per 2 mg of biomolecules. A sample may comprise at most 1 mg of a particle per 1 mg of biomolecules. A sample may comprise at least 1 mg of a particle per 100000 mg of biomolecules. A sample may comprise at least 1 mg of a particle per 10000 mg of biomolecules. A sample may comprise at least 1 mg of a particle per 1000 mg of biomolecules. A sample may comprise at least 1 mg of a particle per 100 mg of biomolecules. A sample may comprise at least 1 mg of a particle per 10 mg of biomolecules. A sample may comprise at least 1 mg of a particle per 2 mg of biomolecules. A sample may comprise at least 1 mg of a particle per 1 mg of biomolecules. A sample may comprise at most 1 mg of a particle per 100000 mg of aggregate protein mass. A sample may comprise at most 1 mg of a particle per 10000 mg of aggregate protein mass. A sample may comprise at most 1 mg of a particle per 1000 mg of aggregate protein mass. A sample may comprise at most 1 mg of a particle per 100 mg of aggregate protein mass. A sample may comprise at most 1 mg of a particle per 10 mg of aggregate protein mass. A sample may comprise at most 1 mg of a particle per 2 mg of aggregate protein mass. A sample may comprise at most 1 mg of a particle per 1 mg of aggregate protein mass. A sample may comprise at least 1 mg of a particle per 100000 mg of aggregate protein mass. A sample may comprise at least 1 mg of a particle per 10000 mg of aggregate protein mass. A sample may comprise at least 1 mg of a particle per 1000 mg of aggregate protein mass. A sample may comprise at least 1 mg of a particle per 100 mg of aggregate protein mass. A sample may comprise at least 1 mg of a particle per 10 mg of aggregate protein mass. A sample may comprise at least 1 mg of a particle per 2 mg of aggregate protein mass. A sample may comprise at least 1 mg of a particle per 1 mg of aggregate protein mass.

A sample may comprise at least 50 cm² particle surface area per mg of biomolecules. A sample may comprise at least 50 cm² particle surface area per mg of protein. A sample may comprise at least 10 cm² particle surface area per mg of biomolecules. A sample may comprise at least 10 cm² particle surface area per mg of protein. A sample may comprise at least 5 cm² particle surface area per mg of biomolecules. A sample may comprise at least 5 cm² particle surface area per mg of protein. A sample may comprise at least 1 cm² particle surface area per mg of biomolecules. A sample may comprise at least 1 cm² particle surface area per mg of protein. A sample may comprise at least 0.5 cm² particle surface area per mg of biomolecules. A sample may comprise at least 0.5 cm² particle surface area per mg of protein. A sample may comprise at least 0.1 cm² particle surface area per mg of biomolecules. A sample may comprise at least 0.1 cm² particle surface area per mg of protein. A sample may comprise at least 0.05 cm² particle surface area per mg of biomolecules. A sample may comprise at least 0.05 cm² particle surface area per mg of protein. A sample may comprise at least 0.01 cm² particle surface area per mg of biomolecules. A sample may comprise at least 0.01 cm² particle surface area per mg of protein. A sample may comprise at least 0.005 cm² particle surface area per mg of biomolecules. A sample may comprise at least 0.005 cm² particle surface area per mg of protein. A sample may comprise at least 0.001 cm² particle surface area per mg of biomolecules. A sample may comprise at least 0.001 cm² particle surface area per mg of protein. A sample may comprise at least 0.0005 cm² particle surface area per mg of biomolecules. A sample may comprise at least 0.0005 cm² particle surface area per mg of protein. A sample may comprise at least 0.0001 cm² particle surface area per mg of biomolecules. A sample may comprise at least 0.0001 cm² particle surface area per mg of protein. A sample may comprise at least 0.00005 cm² particle surface area per mg of biomolecules. A sample may comprise at least 0.00005 cm² particle surface area per mg of protein. A sample may comprise at least 0.00001 cm² particle surface area per mg of biomolecules. A sample may comprise at least 0.00001 cm² particle surface area per mg of protein. A sample may comprise at least 0.000005 cm² particle surface area per mg of biomolecules. A sample may comprise at least 0.000005 cm² particle surface area per mg of protein. A sample may comprise at least 0.000001 cm² particle surface area per mg of biomolecules.

A sample may comprise at most 50 cm² particle surface area per mg of biomolecules. A sample may comprise at most 50 cm² particle surface area per mg of protein. A sample may comprise at most 10 cm² particle surface area per mg of biomolecules. A sample may comprise at most 10 cm² particle surface area per mg of protein. A sample may comprise at most 5 cm² particle surface area per mg of biomolecules. A sample may comprise at most 5 cm² particle surface area per mg of protein. A sample may comprise at most 1 cm² particle surface area per mg of biomolecules. A sample may comprise at most 1 cm² particle surface area per mg of protein. A sample may comprise at most 0.5 cm² particle surface area per mg of biomolecules. A sample may comprise at most 0.5 cm² particle surface area per mg of protein. A sample may comprise at most 0.1 cm² particle surface area per mg of biomolecules. A sample may comprise at most 0.1 cm² particle surface area per mg of protein. A sample may comprise at most 0.05 cm² particle surface area per mg of biomolecules. A sample may comprise at most 0.05 cm² particle surface area per mg of protein. A sample may comprise at most 0.01 cm² particle surface area per mg of biomolecules. A sample may comprise at most 0.01 cm² particle surface area per mg of protein. A sample may comprise at most 0.005 cm² particle surface area per mg of biomolecules. A sample may comprise at most 0.005 cm² particle surface area per mg of protein. A sample may comprise at most 0.001 cm² particle surface area per mg of biomolecules. A sample may comprise at most 0.001 cm² particle surface area per mg of protein. A sample may comprise at most 0.0005 cm² particle surface area per mg of biomolecules. A sample may comprise at most 0.0005 cm² particle surface area per mg of protein. A sample may comprise at most 0.0001 cm² particle surface area per mg of biomolecules. A sample may comprise at most 0.0001 cm² particle surface area per mg of protein. A sample may comprise at most 0.00005 cm² particle surface area per mg of biomolecules. A sample may comprise at most 0.00005 cm² particle surface area per mg of protein. A sample may comprise at most 0.00001 cm² particle surface area per mg of biomolecules. A sample may comprise at most 0.00001 cm² particle surface area per mg of protein. A sample may comprise at most 0.000005 cm² particle surface area per mg of biomolecules. A sample may comprise at most 0.000005 cm² particle surface area per mg of protein. A sample may comprise at most 0.000001 cm² particle surface area per mg of biomolecules.

The ratio between substrate surface area or substrate mass and biomolecule mass may influence the amount of biomolecule recovered from a sample. A biomolecule corona may comprise at most 1% of the biological mass of a biological sample. A biomolecule corona may comprise at most 0.1% of the biological mass of a biological sample. A biomolecule corona may comprise at most 0.01% of the biological mass of a biological sample. A biomolecule corona may comprise at most 0.001% of the biological mass of a biological sample. A biomolecule corona may comprise at most 0.0001% of the biological mass of a biological sample. A biomolecule corona may comprise at most 0.00001% of the biological mass of a biological sample. A biomolecule corona may comprise at most 0.000001% of the biological mass of a biological sample. A biomolecule corona may comprise at most 1% of the protein mass of a biological sample. A biomolecule corona may comprise at most 0.1% of the protein mass of a biological sample. A biomolecule corona may comprise at most 0.01% of the protein mass of a biological sample. A biomolecule corona may comprise at most 0.001% of the protein mass of a biological sample. A biomolecule corona may comprise at most 0.0001% of the protein mass of a biological sample. A biomolecule corona may comprise at most 0.00001% of the protein mass of a biological sample. A biomolecule corona may comprise at most 0.000001% of the protein mass of a biological sample.

A biomolecule corona may comprise at least 1% of the biological mass of a biological sample. A biomolecule corona may comprise at least 0.1% of the biological mass of a biological sample. A biomolecule corona may comprise at least 0.01% of the biological mass of a biological sample. A biomolecule corona may comprise at least 0.001% of the biological mass of a biological sample. A biomolecule corona may comprise at least 0.0001% of the biological mass of a biological sample. A biomolecule corona may comprise at least 0.00001% of the biological mass of a biological sample. A biomolecule corona may comprise at least 0.000001% of the biological mass of a biological sample. A biomolecule corona may comprise at least 1% of the protein mass of a biological sample. A biomolecule corona may comprise at least 0.1% of the protein mass of a biological sample. A biomolecule corona may comprise at least 0.01% of the protein mass of a biological sample. A biomolecule corona may comprise at least 0.001% of the protein mass of a biological sample. A biomolecule corona may comprise at least 0.0001% of the protein mass of a biological sample. A biomolecule corona may comprise at least 0.00001% of the protein mass of a biological sample. A biomolecule corona may comprise at least 0.000001% of the protein mass of a biological sample.

The particles of the present disclosure may be used to serially interrogate a sample (or a portion thereof) by incubating a first particle type with the sample to form a biomolecule corona on the first particle type, separating the first particle type, incubating a second particle type with the sample (or a portion thereof) to form a biomolecule corona on the second particle type, separating the second particle type, and repeating the interrogating (by incubation with the sample) and the separating for any number of particle types. Serial interrogation may also comprise collecting biomolecules of a biomolecule corona from a first particle, and contacting the biomolecules to a second particle to form a second biomolecule corona. In some cases, the biomolecule corona on each particle type used for serial interrogation of a sample may be analyzed by protein corona analysis. The biomolecule content of the supernatant may be analyzed following serial interrogation with one or more particle types.

A particle of the present disclosure may be contacted with a biological sample (e.g., a biofluid) to form a biomolecule corona. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some embodiments, a single particle type (e.g., a particle of a type listed in TABLE 1) may be contacted to a biological sample. In some embodiments, a plurality of particle types (e.g., a plurality of the particle types provided in TABLE 1) may be contacted to a biological sample. The plurality of particle types may be combined and contacted to the biological sample in a single sample volume. The plurality of particle types may be sequentially contacted to a biological sample and separated from the biological sample prior to contacting a subsequent particle type to the biological sample. Protein corona analysis of the biomolecule corona may compress the dynamic range of the analysis compared to a total protein analysis method.

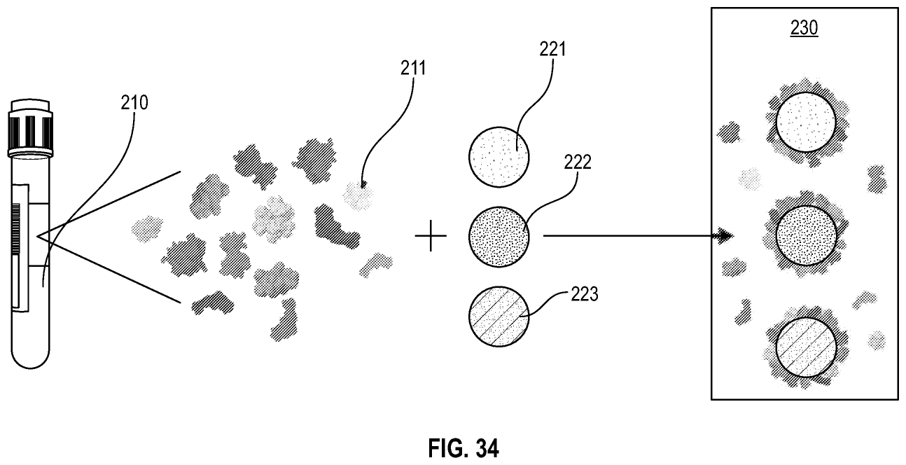

FIG. 34 provides a schematic overview of biomolecule formation, wherein a plurality of particles 221, 222, & 223 particles are contacted with a biological sample 210 comprising biomolecules molecules 211, and wherein each particle adsorbs a plurality of biomolecules from the biological sample to its surface 230. The different particles may be distinct particle types (depicted in the center of the figure, with the top, middle, and bottom spheres representing the three distinct particle types), such that each particle differs from the other particles by at least one physicochemical property. This difference in physicochemical properties can lead to the formation of different protein corona compositions on the particle surfaces.

The composition of the biomolecule corona may depend on a property of the particle. In many cases, the composition of the biomolecule corona is strongly dependent on the surface of the particle. Characteristics such as particle surface material (e.g., ceramic, polymer, metal, metal oxide, graphite, silicon dioxide, etc.), surface texture (rough, smooth, grooved, etc.), surface functionalization (e.g., carboxylate functionalized, amine functionalized, small molecule (e.g., saccharide) functionalized, etc.), shape, curvature, and size can each independently serve as major determinants for biomolecule corona composition. In addition to surface features, the particle core composition, particle density, and particle surface area to mass ratio may each influence biomolecule corona composition. For example, two particles comprising the same surfaces and different cores may form different biomolecule coronas upon contact with the same sample.

Biomolecule corona formation may also be influenced by sample composition. For example, a first sample condition (e.g., low salinity) might favor the solubility of a particular analyte (e.g., an isoform of Bone Morphogenic Protein 1 (BMP1)), and thereby disfavor its binding in a biomolecule corona, while a second sample condition (e.g., high salinity) may diminish the solubility of the analyte, thereby driving its incorporation into a biomolecule corona.

Biomolecule corona composition may also depend on molecular level interactions between the biomolecules themselves. An energetically favorable interaction between two biomolecules may promote their co-incorporation into a biomolecule corona. For example, if a first protein adsorbed to a particle comprises an affinity for a second protein in solution, the first protein may bind to a portion of the second protein, thereby driving its binding to the particle or to other proteins of the biomolecule corona of the particle. A first biomolecule disposed within a biomolecule corona may comprise an energetically unfavorable interaction with a second biomolecule in a biological sample, thereby disfavoring its incorporation into a biomolecule corona. In part owing to these inter-biomolecule dependencies, biomolecule coronas provide sensitive platforms for directly and indirectly sensing biomolecules from a biological sample. For example, detection of a first biomolecule in a biomolecule corona may inform of the presence of a second biomolecule also present in the biomolecule corona.

### Protein Analysis Methods

The particles and methods of use thereof disclosed herein can bind a large number of biomolecules (e.g., proteins) in a biological sample (e.g., a biofluid). For example, a particle disclosed herein can be incubated with a biological sample to form a protein corona comprising at least 5 proteins, at least 10 proteins, at least 15 proteins, at least 20 proteins, at least 25 proteins, at least 30 proteins, at least 40 proteins, at least 50 proteins, at least 60 proteins, at least 80 proteins, 100 proteins, at least 120 proteins, at least 140 proteins, at least 160 proteins, at least 180 proteins, at least 200 proteins, at least 220 proteins, at least 240 proteins, at least 260 proteins, at least 280 proteins, at least 300 proteins, at least 320 proteins, at least 340 proteins, at least 360 proteins, at least 380 proteins, at least 400 proteins, at least 420 proteins, at least 440 proteins, at least 460 proteins, at least 480 proteins, at least 500 proteins, at least 520 proteins, at least 540 proteins, at least 560 proteins, at least 580 proteins, at least 600 proteins, at least 620 proteins, at least 640 proteins, at least 660 proteins, at least 680 proteins, at least 700 proteins, at least 720 proteins, at least 740 proteins, at least 760 proteins, at least 780 proteins, at least 800 proteins, at least 820 proteins, at least 840 proteins, at least 860 proteins, at least 880 proteins, at least 900 proteins, at least 920 proteins, at least 940 proteins, at least 960 proteins, at least 980 proteins, at least 1000 proteins, at least 1100 proteins, at least 1200 proteins, at least 1300 proteins, at least 1400 proteins, at least 1500 proteins, at least 1600 proteins, at least 1800 proteins, at least 2000 proteins, from 100 to 2000 proteins, from 150 to 1500 proteins, from 200 to 1200 proteins, from 250 to 850 proteins, from 300 to 800 proteins, from 350 to 750 proteins, from 400 to 700 proteins, from 450 to 650 proteins, from 500 to 600 proteins, from 200 to 250 proteins, from 250 to 300 proteins, from 300 to 350 proteins, from 350 to 400 proteins, from 400 to 450 proteins, from 450 to 500 proteins, from 500 to 550 proteins, from 550 to 600 proteins, from 600 to 650 proteins, from 650 to 700 proteins, from 700 to 750 proteins, from 750 to 800 proteins, from 800 to 850 proteins, from 850 to 900 proteins, from 900 to 950 proteins, from 950 to 1000 proteins, or over 1000 proteins. In some cases, the median concentration of the biomolecule corona proteins (and thereby the proteins identified in an assay) may be at most 100 μg/mL, at most 200 μg/mL, at most 500 μg/mL, 1 μg/mL, at most 5 μg/mL, at most 10 μg/mL, at most 20 μg/mL, at most 40 μg/mL, at most 100 μg/mL. In some cases, several different types of particles can be used, separately or in combination, to identify large numbers of proteins in a particular biological sample. In other words, particles can be multiplexed in order to bind and identify large numbers of proteins in a biological sample. Protein corona analysis may compress the dynamic range of the analysis compared to a protein analysis of the original sample.

The particle panels disclosed herein can be used to identify the number of distinct proteins disclosed herein, and/or any of the specific proteins disclosed herein, over a wide dynamic range. As used herein, a dynamic range may denote a log 10 value of a ratio of the highest and lowest abundance species of a specified type. Enriching or assaying species over a dynamic range may refer to the abundances of those species in the sample from which they were assayed or derived. For example, the particle panels disclosed herein comprising distinct particle types, can enrich for proteins in a sample, which can be identified using the Proteograph™ workflow, over the entire dynamic range at which proteins are present in a sample (e.g., a plasma sample). In some cases, a particle panel including any number of distinct particle types disclosed herein, enriches and identifies proteins over a dynamic range of at least 2. In some cases, a particle panel including any number of distinct particle types disclosed herein, enriches and identifies proteins over a dynamic range of at least 3. In some cases, a particle panel including any number of distinct particle types disclosed herein, enriches and identifies proteins over a dynamic range of at least 4. In some cases, a particle panel including any number of distinct particle types disclosed herein, enriches and identifies proteins over a dynamic range of at least 5. In some cases, a particle panel including any number of distinct particle types disclosed herein, enriches and identifies proteins over a dynamic range of at least 6. In some cases, a particle panel including any number of distinct particle types disclosed herein, enriches and identifies proteins over a dynamic range of at least 7. In some cases, a particle panel including any number of distinct particle types disclosed herein, enriches and identifies proteins over a dynamic range of at least 8. In some cases, a particle panel including any number of distinct particle types disclosed herein, enriches and identifies proteins over a dynamic range of at least 9. In some cases, a particle panel including any number of distinct particle types disclosed herein, enriches and identifies proteins over a dynamic range of at least 10. In some cases, a particle panel including any number of distinct particle types disclosed herein, enriches and identifies proteins over a dynamic range of at least 11. In some cases, a particle panel including any number of distinct particle types disclosed herein, enriches and identifies proteins over a dynamic range of at least 12. In some cases, a particle panel including any number of distinct particle types disclosed herein, enriches and identifies proteins over a dynamic range of at least 13. In some cases, a particle panel including any number of distinct particle types disclosed herein, enriches and identifies proteins over a dynamic range of at least 14. In some cases, a particle panel including any number of distinct particle types disclosed herein, enriches and identifies proteins over a dynamic range of at least 15. In some cases, a particle panel including any number of distinct particle types disclosed herein, enriches and identifies proteins over a dynamic range of at least 20. In some cases, a particle panel including any number of distinct particle types disclosed herein, enriches and identifies proteins over a dynamic range of from 2 to 100. In some cases, a particle panel including any number of distinct particle types disclosed herein, enriches and identifies proteins over a dynamic range of from 2 to 20. In some cases, a particle panel including any number of distinct particle types disclosed herein, enriches and identifies proteins over a dynamic range of from 2 to 10. In some cases, a particle panel including any number of distinct particle types disclosed herein, enriches and identifies proteins over a dynamic range of from 2 to 5. In some cases, a particle panel including any number of distinct particle types disclosed herein enriches and identifies proteins over a dynamic range of from 5 to 10.

The numbers and types of biomolecules (e.g., proteins) collected in a biomolecule corona may depend on the amount of time a particle is incubated with a sample. In many cases, biomolecule corona formation may have a time dependence, such that different sets of biomolecules collect on a particle at different rates. Further complicating this process, a biomolecule can comprise a time-dependent adsorption or desorption profile. For example, a biomolecule may rapidly collect on a particle during a first phase of biomolecule corona formation, and subsequently slowly desorb from the particle as other biomolecules bind. Accordingly, the length of time over which a particle is contacted to a sample can influence the mass and composition of a resulting biomolecule corona. An assay may generate a biomolecule corona in less than 2 hours. An assay may generate a biomolecule corona in less than 1.5 hours. An assay may generate a biomolecule corona in less than 1 hour. An assay may generate a biomolecule corona in less than 30 minutes. An assay may generate a biomolecule corona in less than 20 minutes. An assay may generate a biomolecule corona in less than 15 minutes. An assay may generate a biomolecule corona in less than 12 minutes. An assay may generate a biomolecule corona in less than 10 minutes. An assay may comprise incubating a particle with a sample for at least 10 minutes to generate a biomolecule corona. An assay may comprise incubating a particle with a sample for at least 12 minutes to generate a biomolecule corona. An assay may comprise incubating a particle with a sample for at least 15 minutes to generate a biomolecule corona. An assay may comprise incubating a particle with a sample for at least 20 minutes to generate a biomolecule corona. An assay may comprise incubating a particle with a sample for at least 30 minutes to generate a biomolecule corona. An assay may comprise incubating a particle with a sample for at least 45 minutes to generate a biomolecule corona. An assay may comprise incubating a particle with a sample for at least 60 minutes to generate a biomolecule corona. An assay may comprise incubating a particle with a sample for at least 90 minutes to generate a biomolecule corona. An assay may comprise incubating a particle with a sample for at least 120 minutes to generate a biomolecule corona.

A biomolecule corona may comprise at least 10⁻¹¹ mg of biomolecules per square millimeter (mm²) of particle surface area. A biomolecule corona may comprise at least 5×10⁻¹¹ mg of biomolecules per square millimeter (mm²) of particle surface area. A biomolecule corona may comprise at least 10⁻¹⁰ mg of biomolecules per square millimeter (mm²) of particle surface area. A biomolecule corona may comprise at least 5×10⁻¹⁰ mg of biomolecules per square millimeter (mm²) of particle surface area. A biomolecule corona may comprise at least 10⁻⁹ mg of biomolecules per square millimeter (mm²) of particle surface area. A biomolecule corona may comprise at least 5×10⁻⁹ mg of biomolecules per square millimeter (mm²) of particle surface area. A biomolecule corona may comprise at least 10⁻⁸ mg of biomolecules per square millimeter (mm²) of particle surface area. A biomolecule corona may comprise at least 5×10⁻⁸ mg of biomolecules per square millimeter (mm²) of particle surface area. A biomolecule corona may comprise at least 10⁻⁷ mg of biomolecules per square millimeter (mm²) of particle surface area. A biomolecule corona may comprise at least 10⁻¹¹ mg of proteins per square millimeter (mm²) of particle surface area. A biomolecule corona may comprise at least 5×10⁻¹¹ mg of proteins per square millimeter (mm²) of particle surface area. A biomolecule corona may comprise at least 10⁻¹⁰ mg of proteins per square millimeter (mm²) of particle surface area. A biomolecule corona may comprise at least 5×10⁻¹⁰ mg of proteins per square millimeter (mm²) of particle surface area. A biomolecule corona may comprise at least 10⁻⁹ mg of proteins per square millimeter (mm²) of particle surface area. A biomolecule corona may comprise at least 5×10⁻⁹ mg of proteins per square millimeter (mm²) of particle surface area. A biomolecule corona may comprise at least 10⁻⁸ mg of proteins per square millimeter (mm²) of particle surface area. A biomolecule corona may comprise at least 5×10⁻⁸ mg of proteins per square millimeter (mm²) of particle surface area. A biomolecule corona may comprise at least 10⁻⁷ mg of proteins per square millimeter (mm²) of particle surface area. A biomolecule corona may comprise an expanded or compressed dynamic range relative to a sample. For example, a biomolecule corona may collect proteins spanning 7 orders of magnitude in concentration in a sample over an abundance range spanning 4 orders of magnitude, thereby compressing the dynamic range of the collected proteins.

Biomolecules collected on a particle may be subjected to further analysis. A method may comprise collecting a biomolecule corona or a subset of biomolecules from a biomolecule corona. The collected biomolecule corona or the collected subset of biomolecules from the biomolecule corona may be subjected to further particle-based analysis (e.g., particle adsorption). The collected biomolecule corona or the collected subset of biomolecules from the biomolecule corona may be purified or fractionated (e.g., by a chromatographic method). The collected biomolecule corona or the collected subset of biomolecules from the biomolecule corona may be analyzed (e.g., by mass spectrometry).

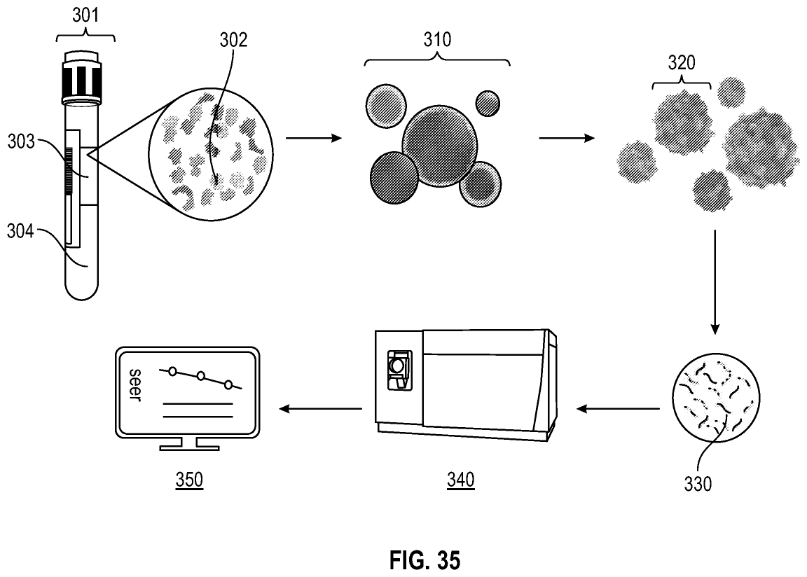

FIG. 35 provides a workflow for a particle-based biomolecule corona (e.g., protein corona) assay consistent with the present disclosure. A biological sample (e.g., human plasma) 301 comprising a plurality of biomolecules 302 may be contacted to a plurality of particles 310. The sample may be treated, diluted, or split into a plurality of fractions 303 and 304 prior to analysis. For example, a whole blood sample may be fractionated into plasma and erythrocyte portions. Upon contact with the particles, a subset or the entirety of the plurality of biomolecules may adsorb to the particles, thereby forming biomolecule coronas 320 bound to the surfaces of the particles. Unbound biomolecules may be separated from the biomolecule coronas (e.g., through wash steps). The biomolecule coronas, or subsets thereof, may be collected from the particles. Alternatively, biomolecules of the biomolecule coronas may be fragmented or chemically treated while bound to the particles. In some assays, biomolecules (e.g., proteins) are fragmented (e.g., digested) while disposed in the biomolecule coronas to yield biomolecule (e.g., peptide) fragments 330. Biomolecules (or their chemically treated or fragmented derivatives) may be analyzed 340, for example by mass spectrometry, to yield data 350 representative of biomolecules 302 from the biological sample 301. The data may be analyzed to identify a biological state of the biological sample.

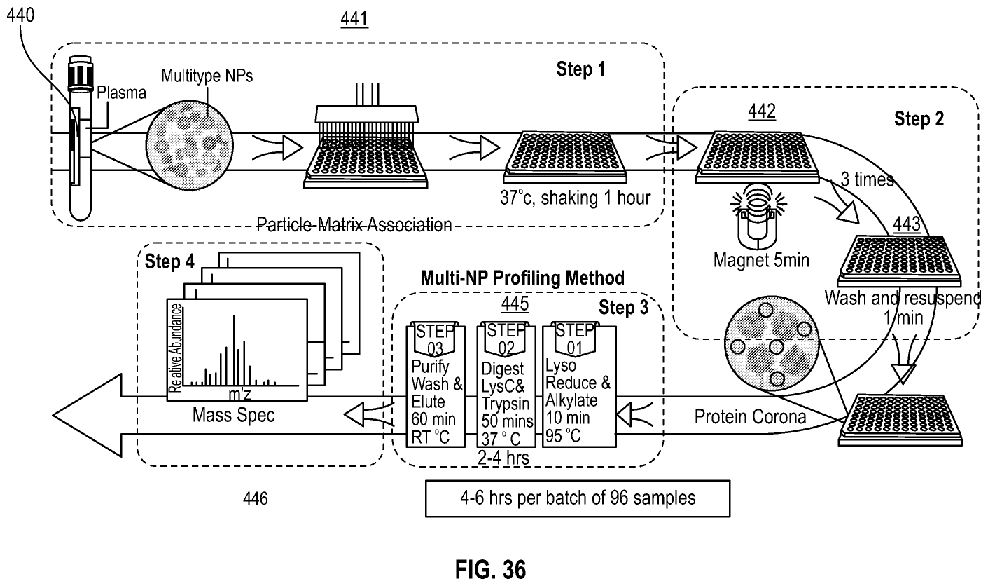

FIG. 36 illustrates an example of a biomolecule corona (e.g., protein corona) analysis workflow consistent with the present disclosure which includes: particle incubation with a biological sample 440 (e.g., plasma), thereby adsorbing biomolecules from the plasma sample to the particles to form biomolecule coronas; partitioning 441 of the particle-plasma sample mixture into a plurality of wells on a 96 well plate; particle collection 442 (e.g., with a magnet); a wash step or plurality of wash steps 443 to remove analytes not adsorbed to the particles; 444 resuspension of the particles and the biomolecules adsorbed thereto; optionally, biomolecule corona digestion or chemical treatment 445 (e.g., protein reduction and digestion); and analysis of the biomolecule coronas or of biomolecules derived therefrom 446 (e.g., by liquid chromatography-mass spectrometry (LC-MS) analysis). While this example provides parallel analyses across 96 well plate wells, a method may comprise a single sample volume or a plurality of sample volumes ranging from two to hundreds of thousands of sample volumes. Furthermore, while this example provides contacting a sample with particles prior to partitioning, a method may alternatively comprise partitioning a sample (e.g., into separate wells of a well plate) prior to contacting with particles. In some cases, sample may be added to partitions comprising particles. For example, a well plate may be provided with particles, buffer, and reagents in dry form, such that a method of use may comprise adding solution to the wells to resuspend the particles and dissolve the buffer and reagents, and then adding sample to the wells.

Protein corona analysis may be fully automated or may comprise an automated component. For example, an automated instrument may contact a sample with a particle or particle panel, identify proteins on the particle or particle panel (e.g., digest the proteins on the particle or particle panel and perform mass spectrometric analysis), and generate data for identifying a specific biomolecule or a biological state of a sample. The automated instrument may divide a sample into a plurality of volumes, and perform analysis on each volume. The automated instrument may analyze multiple separate samples, for example by disposing multiple samples within multiple wells in a well plate, and performing parallel analysis on each sample.

The methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. Thus, the methods disclosed herein provide for isolating or separating a particle type from unbound protein in a sample. A particle type may be separated by a variety of means, including but not limited to magnetic separation, centrifugation, filtration, or gravitational separation. Particle panels may be incubated with a plurality of spatially isolated samples, wherein each spatially isolated sample is in a well in a well plate (e.g., a 96-well plate). After incubation, the particle types in each of the wells of the well plate can be separated from unbound protein present in the spatially isolated samples by placing the entire plate on a magnet. This simultaneously pulls down the superparamagnetic particles in the particle panel. The supernatant in each sample can be removed to remove the unbound protein. These steps (incubate, pull down) can be repeated to effectively wash the particles, thus removing residual background unbound protein that may be present in a sample. This is one example, but one of skill in the art could envision numerous other scenarios in which superparamagnetic particles are rapidly isolated from one or more than one spatially isolated sample at the same time.

The methods and compositions of the present disclosure provide identification and measurement of particular proteins in the biological samples by processing of the proteomic data via digestion of coronas formed on the surface of particles. Examples of proteins that can be identified and measured include highly abundant proteins, proteins of medium abundance, and low-abundance proteins. A low abundance protein may be present in a sample at concentrations at or below about 10 ng/mL. A high abundance protein may be present in a sample at concentrations at or above about 10 μg/mL. A high abundance protein may be present in a sample at concentrations at or above about 1 μM. A high abundance protein may constitute at least 1%, at least 0.1%, or at least 0.05% of the protein mass of a sample. A protein of moderate abundance may be present in a sample at concentrations between about 10 ng/ml and about 10 μg/mL. Examples of proteins that are highly abundant in human plasma include albumin, IgG, and the top 14 proteins in abundance that contribute 95% of the analyte mass in plasma. Additionally, any proteins that may be purified using a conventional depletion column may be directly detected in a sample using the particle panels disclosed herein. Examples of proteins may be any protein listed in published databases such as Keshishian et al. (Mol Cell Proteomics. 2015 September; 14 (9): 2375-93. doi: 10.1074/mcp.M114.046813. Epub 2015 Feb. 27.), Farr et al. (J Proteome Res. 2014 Jan. 3; 13 (1): 60-75. doi: 10.1021/pr4010037. Epub 2013 Dec. 6.), or Pernemalm et al. (Expert Rev Proteomics. 2014 August; 11 (4): 431-48. doi: 10.1586/14789450.2014.901157. Epub 2014 Mar. 24.).

The methods and compositions disclosed herein may also elucidate protein classes or interactions of the protein classes. A protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); or proteins bearing a post-translational modification (e.g., cleavage, N-terminal extension, glycosylation, iodination, acetylation, degradation, acylation, biotinylation, amidation, alkylation, methylation, terminal amino acid cyclization, adenylation, ADP-ribosylation, sulfonation, prenylation, hydroxylation, decarboxylation, glutamylation, glycosylation, isoprenylation, lipoylation, phosphorylation, or sulfurylation). A protein class may contain at least 2 proteins, 5 proteins, 10 proteins, 20 proteins, 40 proteins, 60 proteins, 80 proteins, 100 proteins, 150 proteins, 200 proteins, or more.

The proteomic data of the biological sample can be identified, measured, and quantified using a number of different analytical techniques. For example, proteomic data can be generated using SDS-PAGE or any gel-based separation technique. Peptides and proteins can also be identified, measured, and quantified using an immunoassay, such as ELISA. Alternatively, proteomic data can be identified, measured, and quantified using mass spectrometry, high performance liquid chromatography, LC-MS/MS, Edman Degradation, immunoaffinity techniques, methods disclosed in EP3548652, WO2019083856, WO2019133892, each of which is incorporated herein by reference in its entirety, and other protein separation techniques.

An assay may comprise protein collection of particles, protein digestion, and mass spectrometric analysis (e.g., MS, LC-MS, LC-MS/MS). The digestion may comprise chemical digestion, such as by cyanogen bromide or 2-Nitro-5-thiocyanatobenzoic acid (NTCB). The digestion may comprise enzymatic digestion, such as by trypsin or pepsin. The digestion may comprise enzymatic digestion by a plurality of proteases. The digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. The digestion may cleave peptides at random positions. The digestion may cleave peptides at a specific position (e.g., at methionines) or sequence (e.g., glutamate-histidine-glutamate). The digestion may enable similar proteins to be distinguished. For example, an assay may resolve 8 distinct proteins as a single protein group with a first digestion method, and as 8 separate proteins with distinct signals with a second digestion method. The digestion may generate an average peptide fragment length of 8 to 15 amino acids. The digestion may generate an average peptide fragment length of 12 to 18 amino acids. The digestion may generate an average peptide fragment length of 15 to 25 amino acids. The digestion may generate an average peptide fragment length of 20 to 30 amino acids. The digestion may generate an average peptide fragment length of 30 to 50 amino acids.

An assay may rapidly generate and analyze proteomic data. Beginning with an input biological sample (e.g., a buccal or nasal smear, plasma, or tissue), an assay of the present disclosure may generate and analyze proteomic data in less than 7 hours. Beginning with an input biological sample, an assay of the present disclosure may generate and analyze proteomic data in 5-7 hours. Beginning with an input biological sample, an assay of the present disclosure may generate and analyze proteomic data in less than 5 hours. Beginning with an input biological sample, an assay of the present disclosure may generate and analyze proteomic data in 3-5 hours. Beginning with an input biological sample, an assay of the present disclosure may generate and analyze proteomic data in 2-4 hours. Beginning with an input biological sample, an assay of the present disclosure may generate and analyze proteomic data in 2-3 hours. Beginning with an input biological sample, an assay of the present disclosure may generate and analyze proteomic data in less than 3 hours. Beginning with an input biological sample, an assay of the present disclosure may generate and analyze proteomic data in less than 2 hours. The analyzing may comprise identifying a protein group. The analyzing may comprise identifying a protein class. The analyzing may comprise quantifying an abundance of a biomolecule, a peptide, a protein, protein group, or a protein class. The analyzing may comprise identifying a ratio of abundances of two biomolecules, peptides, proteins, protein groups, or protein classes. The analyzing may comprise identifying a biological state.

### Measurement

In some embodiments, a measurement can be preceded by binding a plurality of molecules to a surface. The surface can comprise a sensor element surface. The sensor element surface can comprise a particle surface. The particle surface can be a nanoparticle surface. The particle surface can be a microparticle surface. The particle surface can comprise pores. The binding can comprise adsorption. The binding can be non-specific. The binding can be specific. The plurality of molecules can form a corona on the particle surface.

In some embodiments, measured quantities comprise measured intensities. In some embodiments, in-sample quantities comprise measured intensities. The measured intensities can be obtained using a variety of methods and/or instrumentation. The measured intensities can comprise mass spectrometry (MS) intensities. The MS intensities can comprise peptide intensities, protein group intensities, or both. The MS intensities can comprise small molecule intensities. The MS intensities can be based on data-independent acquisition (DIA) MS, data-dependent acquisition (DDA) MS, or both. The MS intensities can be based on liquid-chromatography tandem mass spectrometry (LC-MS/MS). The measured intensities can be obtained using a nanopore sensor. The measured intensities can be obtained using an immunoassay. The measured intensities can be obtained using a nucleic acid sequencer. The measured intensities can comprise fluorescence signals. The measured intensities can comprise an induced current. In some embodiments, the measured intensities can be obtained using gas phase separation.

The measured intensities can be obtained using an antibody. The measured intensities can be obtained by binding a molecule in the plurality of molecules to an antibody. The measured intensities can be obtained by binding the molecule to a pair of antibodies. The pair of antibodies can comprise complementary single-stranded nucleic acid sequences attached thereto. When the pair of antibodies bind to the molecule, the complementary nucleic acids can hybridize to form a double stranded nucleic acid. The double stranded nucleic acid can be configured to form a binding complex with a polymerase and a plurality of nucleotides, nucleosides, nucleotide analogs, and/or nucleoside analogs to perform an amplification reaction to produce a detectable signal.

The measured intensities can be obtained using an aptamer. The aptamer can be coupled to a surface via a cleavable linker. The surface can be a particle surface. The cleavable linker can be photocleavable. The measured intensities can be obtained by contacting the molecule and the aptamer with a macromolecular competitor configured to, in a fluid composition, reduce dissociation of a complex comprising the one or more aptamers and the molecule. The macromolecular competitor can be a polyanionic macromolecule.

The measured intensities can be obtained using protein sequencing. The protein sequencing can comprise digesting the plurality of proteins to generate a plurality of protein fragments. The protein sequencing can comprise immobilizing the plurality of protein fragments to a semiconductor substrate. The protein sequencing can comprise contacting the plurality of protein fragments with a plurality of labeled recognizers. The plurality of labeled recognizers can be configured to attach to a predetermined chemical moiety in the plurality of protein fragments at the N-terminus of the plurality of protein fragments. The protein sequencing can comprise exciting the plurality of labeled recognizers to detect the plurality of labeled recognizers, thereby detecting the predetermined chemical moiety. The protein sequencing can comprise removing an amino acid from the N-terminus of the plurality of protein fragments. The protein sequencing can comprise contacting the plurality of protein fragments with a second plurality of labeled recognizers. The protein sequencing can comprise exciting the second plurality of labeled recognizers to detect a second amino acid from the N-terminus of the plurality of protein fragments, thereby performing the protein sequencing.

In some embodiments, the measured intensities can be obtained using a neat measurement condition. In some embodiments, the neat measurement condition does not comprise binding the molecule to the surface. In some embodiments, the measured intensities can be obtained using liquid chromatography mass spectrometry (LC-MS) with a gradient length equal to or greater than 10, 20, 30, 40, 50, 60, 70, 80, 90, 100, 110, or 120 minutes. In some embodiments, the measured intensities can be obtained using LC-MS with a gradient length less than or equal to 10, 20, 30, 40, 50, 60, 70, 80, 90, 100, 110, or 120 minutes.

In some embodiments, a machine learning algorithm is trained using an input dataset. The input dataset can comprise the quantities, the in-sample quantities, a plurality of differences between the quantities and the in-sample quantities, or any combination thereof

The in-sample quantities can be measured in various ways. In some embodiments, the in-sample quantities comprise average abundance values of the molecules over a plurality of samples. In some embodiments, the average abundance values are concentration values, intensities values, or relative abundance values. In some embodiments, the in-sample quantities comprise an aggregate of measurements of samples.

In some cases, the in-sample quantities can be obtained from databases. For example, the reference quantities can be obtained from the Human Plasma Proteome Project (HPPP) or the Proteomics Identifications Database (PRIDE).

In some cases, the in-sample quantities can be obtained from labeled molecules in a sample. For example, proteins adsorbed on the surface can be labeled with tandem-mass-tag (TMT; e.g., isobaric or non-isobaric labeling such as iTRAQ) and be mixed with TMT labeled proteins obtained from a neat extraction (e.g., proteins without contacting with a surface). In some cases, a sample of known composition can be labeled (e.g., via Stable Isotope Labeling by Amino Acids in Cell Culture, “SILAC”) and be mixed with proteins adsorbed on the surface. Signals obtained from the in-sample quantities (e.g., quantities of proteins from a sample of known composition, or quantities of proteins measured from a neat extraction method) can be used.

Quantities of a biomolecule or biomolecule group can be obtained using different physicochemical parameters. The one or more physicochemical parameters can comprise: sample to surface ratio, incubation time, pH, salt concentration, ionic strength, solvent composition, solvent dielectric constant, crowding agent concentration, temperature, sample composition, surfactant concentration, concentration of enzymes, activity of enzymes, chemical reactions, concentrations of small molecules, surface chemistry (e.g., hydrophobicity, charge, polymeric, chemical moieties, etc.) or any combination thereof. The sample to surface ratio can comprise (i) volume of sample to surface area of the surface, (ii) volume of sample to mass of a substrate comprising the surface, (iii) mass of sample to surface area of the surface, or (iv) mass of sample to mass of the substrate comprising the surface. The one or more physicochemical parameters can comprise a ratio of surface area of the surface to a volume of a sample comprising the plurality of molecules. The ratio can be at least 0.0001, 0.0005, 0.001, 0.005, 0.01, 0.05, 0.1, 0.5, 1, 5, or 10 cm² per μL. The ratio can be at most 0.0001, 0.0005, 0.001, 0.005, 0.01, 0.05, 0.1, 0.5, 1, 5, or 10 cm² per μL. The one or more physicochemical parameters can comprise a ratio of surface area of the surface to a concentration of the plurality of molecules in a sample. The ratio can be at least 0.0001, 0.0005, 0.001, 0.005, 0.01, 0.05, 0.1, 0.5, 1, 5, or 10 cm² per μg/μL. The ratio can be at most 0.0001, 0.0005, 0.001, 0.005, 0.01, 0.05, 0.1, 0.5, 1, 5, or 10 cm² per μg/μL. The one or more physicochemical parameters can comprise a ratio of surface area of the surface to a mass of the plurality of molecules in a sample. The ratio can be at least 0.0001, 0.0005, 0.001, 0.005, 0.01, 0.05, 0.1, 0.5, 1, 5, or 10 cm² per μg. The ratio can be at most 0.0001, 0.0005, 0.001, 0.005, 0.01, 0.05, 0.1, 0.5, 1, 5, or 10 cm² per μg. The one or more physicochemical parameters can comprise a ratio of mass of a substrate comprising the surface to a volume of a sample comprising the plurality of molecules. The ratio can be at least 0.0001, 0.0005, 0.001, 0.005, 0.01, 0.05, 0.1, 0.5, 1, 5, or 10 μg/μL. The ratio can be at most 0.0001, 0.0005, 0.001, 0.005, 0.01, 0.05, 0.1, 0.5, 1, 5, or 10 μg/μL. The one or more physicochemical parameters can comprise a ratio of mass of a substrate comprising the surface to a concentration of the plurality of molecules in a sample. The ratio can be at least 0.0001, 0.0005, 0.001, 0.005, 0.01, 0.05, 0.1, 0.5, 1, 5, or 10 μL⁻¹. The ratio can be at most 0.0001, 0.0005, 0.001, 0.005, 0.01, 0.05, 0.1, 0.5, 1, 5, or 10 μL⁻¹. The one or more physicochemical parameters can comprise a ratio of mass of a substrate comprising the surface to a mass of the plurality of molecules in a sample. The ratio can be at least 0.0001, 0.0005, 0.001, 0.005, 0.01, 0.05, 0.1, 0.5, 1, 5, or 10. The ratio can be at most 0.0001, 0.0005, 0.001, 0.005, 0.01, 0.05, 0.1, 0.5, 1, 5, or 10.

The one or more physicochemical parameters can comprise an incubation time for the plurality of molecules to the surface. The incubation time can be at least 1, 15, 30, 45, or 60 seconds. The incubation time can be at least 1, 15, 30, or 60 minutes. The incubation time can be at least 1, 2, 3, 4, 8, 12, 16, 20, or 24 hours. The incubation time can be at least 1, 2, 3, 4, 5, 6 or 7 days. The incubation time can be at most 1, 2, 3, 4, 5, 6 or 7 days. The incubation time can be at most 1, 2, 3, 4, 8, 12, 16, 20, or 24 hours. The incubation time can be at most 1, 15, 30, or 60 minutes. The incubation time can be at most 1, 15, 30, or 60 seconds.

The pH can be at least 1, 2, 3, 4, 5, 6, 7, 8, 9, 10, 11, 12, 13, or 14. The pH can be at most 1, 2, 3, 4, 5, 6, 7, 8, 9, 10, 11, 12, 13, or 14. The ion concentration can be at least 0.1, 0.2, 0.3, 0.4, 0.5, 0.6, 0.7, 0.8, 0.9, 1, 1.5, 2, 2.5, 3, 3.5, 4, 4.5, or 5 mols per liter. The ion concentration can be at most 0.1, 0.2, 0.3, 0.4, 0.5, 0.6, 0.7, 0.8, 0.9, 1, 1.5, 2, 2.5, 3, 3.5, 4, 4.5, or 5 mols per liter. In some cases, a solvent can comprise a salt comprising LiF, LiCl, LiBr, LiI, Li₂SO₄, BeF₂, BeCl₂, BeBr₂, BeI₂, BeSO₄, NaF, NaCl, NaBr, NaI, Na₂SO₄, MgF₂, MgCl₂, MgBr₂, MgI₂, MgSO₄, KF, KCl, KBr, KI, K₂SO₄, CaF₂, CaCl₂), CaBr₂, CaI₂, KSO₄, NH₄F, NH₄Cl, NH₄Br, NH₄I, (NH₄)₂SO₄, or any combination thereof. The solvent can comprise water, alcohol, ketone, a buffer, or any combination thereof. In some cases, a solvent may comprise various acids or bases. In some cases, an acid may comprise hydrochloric, acetic acid, sulfuric acid, nitric acid, citric acid, or any combination thereof. In some cases, a base may comprise NaOH, KOH, Ca(OH)₂, NH₄OH, or any combination thereof. The solvent dielectric constant can be at least 1, 5, 10, 15, 20, 25, 30, 35, 40, 45, 50, 55, 60, 65, 70, 75, or 80. The solvent dielectric constant can be at most 1, 5, 10, 15, 20, 25, 30, 35, 40, 45, 50, 55, 60, 65, 70, 75, or 80. The temperature can be at least-20, −15, −10, −5, 0, 5, 10, 15, 20, 25, 30, 35, 40, 45, 50, 55, 65, 70, 75, 80, 85, 90, 95, or 100° C. The temperature can be at most-20, −15, −10, −5, 0, 5, 10, 15, 20, 25, 30, 35, 40, 45, 50, 55, 65, 70, 75, 80, 85, 90, 95, or 100° C.

Measurements can be obtained in serial, in parallel, or a combination thereof. For example, a plurality of partitions or wells can be provided, wherein one of the partitions or the wells can be configured to provide a different physicochemical condition for performing the measurement compared to another. One partition or well can comprise a different solvent, temperature, sample to surface ratio, be used with a different incubation time, etc., compared to another partition or well. In some embodiments, a control sample (e.g., a plasma standard sample) can be provided in a partition or a well.

### Machine Learning

A wide variety of supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive Bayes, linear regression, polynomial regression, SVM for regression, K-means clustering, and hidden Markov models, among others can be used to adjust the measured quantity of a biomolecule or biomolecule group. In some embodiments, a machine learning algorithm can be used to adjust the measured quantity of a biomolecule or biomolecule group.

Input features to a machine learning algorithm may comprise various kinds of information. In some cases, an input feature may comprise a value that represents a physicochemical property of a surface used to assay a biomolecule. A physicochemical property of a particle may comprise various properties disclosed herein, which includes: charge, hydrophobicity, hydrophilicity, amphipathicity, coordinating, reaction class, surface free energy, various functional groups/modifications (e.g., sugar, polymer, amine, amide, epoxy, crosslinker, hydroxyl, aromatic, or phosphate groups). In some cases, an input feature may comprise a value that represents a parameter of a given measurement. A parameter may comprise incubation conditions including temperature, incubation time, pH, buffer type, and any variables in performing a measurement disclosed herein. In some embodiments, the input datasets may include a series of quantity measurements at different conditions.

In some embodiments, a machine learning algorithm can be a clustering algorithm. A clustering algorithm can refer to a method of grouping samples in a dataset by some measure of similarity. In some cases, samples can be grouped in a set space, for example, element ‘a’ is in set ‘A’. In some cases, samples can be grouped in a continuous space, for example, element ‘a’ is a point in Euclidean space with distance ‘l’ away from the centroid of elements comprising cluster ‘A’. In some cases, samples can be grouped in a graph space, for example, element ‘a’ is highly connected to elements comprising cluster ‘A’. In some cases, clustering can refer to the principle of organizing a plurality of elements into groups in some mathematical space based on some measure of similarity.

In some cases, clustering can comprise grouping any number of biomolecules or quantities of biomolecules in a dataset by any quantitative measure of similarity. In some cases, clustering can comprise K-means clustering. In some cases, clustering can comprise hierarchical clustering. In some cases, clustering can comprise using random forest models. In some cases, clustering can comprise boosted tree models. In some cases, clustering can comprise using support vector machines. In some cases, clustering can comprise calculating one or more N-1 dimensional surfaces in N-dimensional space that partitions a dataset into clusters. In some cases, clustering can comprise distribution-based clustering. In some cases, clustering can comprise fitting a plurality of prior distributions over the data distributed in N-dimensional space. In some cases, clustering can comprise using density-based clustering. In some cases, clustering can comprise using fuzzy clustering. In some cases, clustering can comprise computing probability values of a data point belonging to a cluster. In some cases, clustering can comprise using constraints. In some cases, clustering can comprise using supervised learning. In some embodiments, clustering can comprise using unsupervised learning.

In some cases, clustering can comprise grouping molecules based on similarity. In some cases, clustering can comprise grouping molecules based on quantitative similarity. In some cases, clustering can comprise grouping molecules based on one or more features of each molecule. In some cases, clustering can comprise grouping molecules based on one or more labels of each molecule. In some cases, clustering can comprise grouping molecules based on Euclidean coordinates in a numerical representation of molecules. In some cases, clustering can comprise grouping molecules based on protein structural groups or functional groups (e.g., protein structures, substructures, or functional groups from protein databases such as Protein Data Bank or CATH Protein Structure Classification database). In some cases, a protein structural group or functional group may comprise protein primary structure, secondary structure, tertiary structure, or quaternary structure. In some cases, a protein structural group or functional group may be based at least partially on alpha helices, beta sheets, relative distribution of amino acids with different properties (e.g., aliphatic, aromatic, hydrophilic, acidic, basic, etc.), structural families (e.g., TIM barrel and beta barrel fold), protein domains (e.g., Death effector domain). In some cases, a protein structural group or functional group may be based at least partially on functional or spatial properties (e.g., functional groups-group of immune globulins, cytokines, cytoskeletal biomolecules, etc.).

When trained, the machine learning algorithm can generate an output value that can be a normalization value for adjusting the quantities of the plurality of molecules. The normalization value can be the difference between a quantity and an in-sample quantity. The normalization value can be a ratio between a quantity and an in-sample quantity. When trained, the machine learning algorithm can generate an output value that is an adjusted quantity. A trained machine learning algorithm can be used to generate an adjusted quantity of a molecule at an in-sample condition using a measured quantity of the molecule at another condition.

A trained machine learning algorithm can be fine-tuned with additional datasets. A new input dataset can be provided, wherein the input dataset comprises features obtained for molecules in a condition different from the conditions in the initial training dataset. The new input dataset can comprise molecules in common with the initial training dataset, or no molecules in common. The new input dataset may be based on a different type of sample compared to the initial training dataset.

Adjusted quantities of the molecules can be more accurate or closer to the actual quantities in a sample, compared to the initially measured quantities. The adjusted quantities can be on average more accurate by at least 1, 2, 3, 4, 5, 6, 7, 8, 9, 10, 15, 20, 25, 30, 35, 40, 45, or 50 percent. The adjusted quantities can be more accurate by at most 1, 2, 3, 4, 5, 6, 7, 8, 9, 10, 15, 20, 25, 30, 35, 40, 45, or 50 percent. In some embodiments, the adjusted quantities are on average at least 10 percent more accurate. In some embodiments, the adjusted quantities are on average at least 20 percent more accurate. The average can be a mean or a median.

A coefficient of determination between the adjusted quantities and the in-sample quantities of the plurality of molecules can be at least 0.7, 0.8, 0.85, 0.9, 0.91, 0.92, 0.93, 0.94, 0.95, 0.96, 0.97, 0.98, or 0.99, when the coefficient of determination is measured with a k-fold cross validation, wherein k is an integer greater than 1. A coefficient of determination between the adjusted quantities and the in-sample quantities of the plurality of molecules can be at most 0.7, 0.8, 0.85, 0.9, 0.91, 0.92, 0.93, 0.94, 0.95, 0.96, 0.97, 0.98, or 0.99, when the coefficient of determination is measured with a k-fold cross validation, wherein k is an integer greater than 1. The k can be at least 2, 3, 4, 5, 6, 7, 8, 9, 10, 20, 30, 40, 50, 60, 70, 80, 90, or 100. The k can be at most 2, 3, 4, 5, 6, 7, 8, 9, 10, 20, 30, 40, 50, 60, 70, 80, 90, or 100. A mean absolute error (MAE) between the adjusted quantities and the in-sample quantities of the plurality of molecules can be at most 1, 2, 3, 4, 5, 6, 7, 8, 9, 10, 15, 20, 25, or 30 percent of the standard deviation of the in-sample quantities when the MAE is measured with a k-fold cross validation, wherein k is an integer greater than 1. A MAE between the adjusted quantities and the in-sample quantities of the plurality of molecules can be at least 1, 2, 3, 4, 5, 6, 7, 8, 9, 10, 15, 20, 25, or 30 percent of the standard deviation of the in-sample quantities when the MAE is measured with a k-fold cross validation, wherein k is an integer greater than 1. The k can be at least 2, 3, 4, 5, 6, 7, 8, 9, 10, 20, 30, 40, 50, 60, 70, 80, 90, or 100. The k can be at most 2, 3, 4, 5, 6, 7, 8, 9, 10, 20, 30, 40, 50, 60, 70, 80, 90, or 100.

### Dynamic Range

The biomolecule corona analysis methods described herein may comprise assaying biomolecules in a sample of the present disclosure across a wide dynamic range. The dynamic range of biomolecules assayed in a sample may be a range of measured signals of biomolecule abundances as measured by an assay method (e.g., mass spectrometry, chromatography, gel electrophoresis, spectroscopy, or immunoassays) for the biomolecules contained within a sample. For example, an assay capable of detecting proteins across a wide dynamic range may be capable of detecting proteins of very low abundance to proteins of very high abundance. The dynamic range of an assay may be directly related to the slope of assay signal intensity as a function of biomolecule abundance. For example, an assay with a low dynamic range may have a low (but positive) slope of the assay signal intensity as a function of biomolecule abundance, e.g., the ratio of the signal detected for a high abundance biomolecule to the ratio of the signal detected for a low abundance biomolecule may be lower for an assay with a low dynamic range than an assay with a high dynamic range. In specific cases, dynamic range may refer to the dynamic range of proteins within a sample or assaying method.

The biomolecule corona analysis methods described herein may compress the dynamic range of an assay. The dynamic range of an assay may be compressed relative to another assay if the slope of the assay signal intensity as a function of biomolecule abundance is lower than that of the other assay. For example, a plasma sample assayed using protein corona analysis with mass spectrometry may have a compressed dynamic range compared to a plasma sample assayed using mass spectrometry alone, directly on the sample or compared to provided abundance values for plasma proteins in databases (e.g., the database provided in Keshishian et al., Mol. Cell Proteomics 14, 2375-2393 (2015), also referred to herein as the “Carr database”). The compressed dynamic range may enable the detection of more low abundance biomolecules in a biological sample using biomolecule corona analysis with mass spectrometry than using mass spectrometry alone.

In some cases, the dynamic range of a proteomic analysis assay may be the ratio of the signal produced by highest abundance proteins (e.g., the highest 10% of proteins by abundance) to the signal produced by the lowest abundance proteins (e.g., the lowest 10% of proteins by abundance). Compressing the dynamic range of a proteomic analysis may comprise decreasing the ratio of the signal produced by the highest abundance proteins to the signal produced by the lowest abundance proteins for a first proteomic analysis assay relative to that of a second proteomic analysis assay. The protein corona analysis assays disclosed herein may compress the dynamic range relative to the dynamic range of a total protein analysis method (e.g., mass spectrometry, gel electrophoresis, or liquid chromatography).

Provided herein are several methods for compressing the dynamic range of a biomolecular analysis assay to facilitate the detection of low abundance biomolecules relative to high abundance biomolecules. For example, a particle type of the present disclosure can be used to serially interrogate a sample. Upon incubation of the particle type in the sample, a biomolecule corona comprising forms on the surface of the particle type. If biomolecules are directly detected in the sample without the use of said particle types, for example by direct mass spectrometric analysis of the sample, the dynamic range may span a wider range of concentrations, or more orders of magnitude, than if the biomolecules are directed on the surface of the particle type. Thus, using the particle types disclosed herein may be used to compress the dynamic range of biomolecules in a sample. Without being limited by theory, this effect may be observed due to more capture of higher affinity, lower abundance biomolecules in the biomolecule corona of the particle type and less capture of lower affinity, higher abundance biomolecules in the biomolecule corona of the particle type.

A dynamic range of a proteomic analysis assay may be the slope of a plot of a protein signal measured by the proteomic analysis assay as a function of total abundance of the protein in the sample. Compressing the dynamic range may comprise decreasing the slope of the plot of a protein signal measured by a proteomic analysis assay as a function of total abundance of the protein in the sample relative to the slope of the plot of a protein signal measured by a second proteomic analysis assay as a function of total abundance of the protein in the sample. The protein corona analysis assays disclosed herein may compress the dynamic range relative to the dynamic range of a total protein analysis method (e.g., mass spectrometry, gel electrophoresis, or liquid chromatography).

### Kits

Provided herein are kits comprising compositions of the present disclosure that may be used to perform the methods of the present disclosure. A kit may comprise one or more particle types to interrogate a sample to identify a biological state of a sample. In some cases, a kit may comprise a particle type provided in TABLE 1. A kit may comprise a reagent for functionalizing a particle (e.g., a reagent for tethering a small molecule functionalization to a particle surface). The kit may be pre-packaged in discrete aliquots. In some cases, the kit can comprise a plurality of different particle types that can be used to interrogate a sample. The plurality of particle types can be pre-packaged where each particle type of the plurality is packaged separately. Alternately, the plurality of particle types can be packaged together to contain combination of particle types in a single package. A particle may be provided in dried (e.g., lyophilized) form, or may be provided in a suspension or solution. The particles may be provided in a well plate. For example, a kit may contain an 8 well plate, an 8-384 well plate with particles provided (e.g., sealed) within the wells. For example, a well plate may comprise at least 8, at least 16, at least 24, at least 32, at least 40, at least 48, at least 56, at least 64, at least 72, at least 80, at least 88, at least 96, at least 104, at least 112, at least 120, at least 128, at least 136, at least 144, at least 152, at least 160, at least 168, at least 176, at least 184, at least 192, at least 200, at least 208, at least 216, at least 224, at least 232, at least 240, at least 248, at least 256, at least 264, at least 272, at least 280, at least 288, at least 296, at least 304, at least 312, at least 320, at least 328, at least 336, at least 344, at least 352, at least 360, at least 368, at least 376, at least 384, at least 392, at least 400 wells comprising particles. Two wells in such a well plate may contain different particles or different concentrations of particles. Two wells may comprise different buffers or chemical conditions. For example, a well plate may be provided with different particles in each row of wells and different buffers in each column of rows. A well may be sealed by a removable covering. For example, a kit may comprise a well plate comprising aplastic slip covering a plurality of wells. A well may be sealed by a pierceable covering. For example, a well may be covered by a septum that a needle can pierce to facilitate sample movement into and out of the well.

A kit may comprise a composition and/or instructions for generating a peptide surface functionalization on a particle. The kit may comprise a reagent for attaching a peptide to the surface of a particle. The reagent may activate a surface functionalization or a portion of a surface of a particle to react with a peptide or a linker. The reagent may activate a peptide to react with a particle, a surface functionalization of a particle, or a linker. For example, the reagent may chemically modify and enhance the electrophilicity of C-terminal residues of peptides to facilitate their coupling to particle-derived amines. The kit may comprise a linker comprising a first moiety capable of coupling to a site on a particle and a second moiety capable of coupling to a site on a peptide. The kit may comprise an affinity binding reagent, such as streptavidin, coupled or configured to couple to a particle or peptide, and a ligand, such as biotin, coupled or configured to couple to a peptide.

The kit may comprise a reagent or composition for generating a plurality of peptides. For example, a kit may comprise a protease for generating oligopeptides from a protein sample, as well as a means for coupling the oligopeptides generated therefrom to a particle. The kit may comprise reagents for de novo peptide synthesis, for example a plurality of α-carboxylate activated (e.g., TMS-derivatized) amino acids for stepwise peptide synthesis.

The kit may comprise a reagent for functionalizing a peptide, such as a peptide coupled to the surface of a particle. The reagent may chemically modify the peptide at a specific residue or moiety (e.g., a reagent may phosphorylate tyrosine residues of particle-bound peptides). The reagent may cleave the peptide in a sequence specific or non-specific manner. The reagent may couple a first peptide to a second peptide.

### Sample Collection and Extraction Methods

A variety of samples may be assayed in accordance with the methods and compositions of this disclosure. The samples disclosed herein may be analyzed by biomolecule corona analysis after serially interrogating the sample with various types of substrates. In some embodiments, a sample may be fractioned prior to protein corona analysis. In some embodiments, a sample may be depleted prior to biomolecule corona analysis. In some embodiments, a method of this disclosure may comprise contacting a sample with one or more particle types and performing a biomolecule corona analysis on the sample.

A sample may be a biological sample. For example, a biological sample may be a biofluid sample such as cerebrospinal fluid (CSF), synovial fluid (SF), urine, plasma, serum, tear, crevicular fluid, semen, whole blood, milk, nipple aspirate, ductal lavage, vaginal fluid, nasal fluid, ear fluid, gastric fluid, pancreatic fluid, trabecular fluid, lung lavage, prostatic fluid, sputum, fecal matter, bronchial lavage, fluid from swabbings, bronchial aspirants, sweat or saliva. A biofluid may be a fluidized solid, for example a tissue homogenate, or a fluid extracted from a biological sample. A biological sample may be, for example, a tissue sample or a fine needle aspiration (FNA) sample. In some embodiments a biological sample may be a cell culture sample. For example, a sample that may be used in the methods disclosed herein can either include cells grow in cell culture or can include acellular material taken from cell cultures. In some embodiments, a biofluid is a fluidized biological sample. For example, a biofluid may be a fluidized cell culture extract. In some embodiments, a sample may be extracted from a fluid sample, or a sample may be extracted from a solid sample. For example, a sample may comprise gaseous molecules extracted from a fluidized solid (e.g., a volatile organic compound).

The biomolecule corona analysis methods described herein may comprise assaying proteins in a sample of the present disclosure across a wide dynamic range. The dynamic range of biomolecules assayed in a sample may be a range of measured signals of biomolecule abundances as measured by an assay method (e.g., mass spectrometry, chromatography, gel electrophoresis, spectroscopy, or immunoassays) for the biomolecules contained within a sample. For example, an assay capable of detecting proteins across a wide dynamic range may be capable of detecting proteins of very low abundance to proteins of very high abundance. The dynamic range of an assay may be directly related to the slope of assay signal intensity as a function of biomolecule abundance. For example, an assay with a low dynamic range may have a low (but positive) slope of the assay signal intensity as a function of biomolecule abundance, e.g., the ratio of the signal detected for a high abundance biomolecule to the ratio of the signal detected for a low abundance biomolecule may be lower for an assay with a low dynamic range than an assay with a high dynamic range. The biomolecule corona analysis methods described herein may compress the dynamic range of an assay. The dynamic range of an assay may be compressed relative to another assay if the slope of the assay signal intensity as a function of biomolecule abundance is lower than that of the other assay. For example, a plasma sample assayed using biomolecule corona analysis with mass spectrometry may have a compressed dynamic range compared to a plasma sample assayed using mass spectrometry alone, directly on the sample or compared to provided abundance values for plasma biomolecules in databases (e.g., the database provided in Keshishian et al., Mol. Cell Proteomics 14, 2375-2393 (2015), also referred to herein as the “Carr database”). The compressed dynamic range may enable the detection of more low abundance biomolecules in the plasma sample using biomolecule corona analysis with mass spectrometry than using mass spectrometry alone.

Compression of a dynamic range of an assay may enable the detection of low abundance biomolecules using the methods disclosed herein (e.g., serial interrogation with a particle followed by an assay for quantitating protein abundance such as mass spectrometry). For example, an assay (e.g., mass spectrometry) may be capable of detecting a dynamic range of 3 orders of magnitude. In a sample comprising five proteins, A, B, C, D, and E, in abundances of 1 ng/mL, 10 ng/ml, 100 ng/mL, 1,000 ng/mL, and 10,000 ng/mL, respectively, the assay (e.g., mass spectrometry) may detect proteins B, C, D, and E. However, using the methods disclosed herein of incubating the sample with a particle, proteins A, B, C, D, and E may have different affinities for the particle surface and may adsorb to the surface of the particle to form the biomolecule corona at different abundancies than present in the sample. For example, proteins A, B, C, D, and E may be present in the biomolecule corona at abundancies of 1 ng/ml, 231 ng/ml, 463 ng/mL, 694 ng/mL, and 926 ng/mL, respectively. Thus, using the particles disclosed herein in methods of interrogating a sample results in compressing the dynamic range to 2 orders of magnitude and the resulting assay (e.g., mass spectrometry) can detect all five proteins.

In some aspects, the dynamic range of the plurality of biomolecules in the first biomolecule corona is a first ratio of: a) a signal produced by a higher abundance biomolecules of the plurality of biomolecules in the first biomolecule corona; and b) a signal produced by a lower abundance biomolecule of the plurality of biomolecules in the first biomolecule corona. In some aspects, the dynamic range of the plurality of biomolecules in the first biomolecule corona is a first ratio of a concentration of the highest abundance biomolecule to a concentration of the lowest abundance biomolecule in the plurality of proteins in the first biomolecule corona. In some aspects, the dynamic range of the plurality of biomolecules in the first biomolecule corona is a first ratio of a top decile of biomolecules to a bottom decile of biomolecules in the plurality of proteins in the first biomolecule corona. In some aspects, the dynamic range of the plurality of biomolecules in the first biomolecule corona is a first ratio comprising a span of the interquartile range of biomolecules in the plurality of biomolecules in the first biomolecule corona. In some aspects, the dynamic range of the plurality of biomolecules in the first biomolecule corona is a first ratio comprising a slope of fitted data in a plot of all concentrations of biomolecules in the plurality of biomolecules in the first biomolecule corona versus known concentrations of the same biomolecules in the sample.

In some aspects, the dynamic range of the plurality of biomolecules in the sample, as measured by a total biomolecule analysis method (e.g., a total protein analysis method), is a second ratio comprising a span of the interquartile range of biomolecules in the plurality of biomolecules in the sample. In some aspects, the dynamic range of the plurality of biomolecules in the sample, as measured by a total biomolecule analysis method, is a second ratio comprising a slope of fitted data in a plot of all concentrations of biomolecules in the plurality of biomolecules in the sample versus known concentrations of the same biomolecules in the sample. In some aspects, the known concentrations of the same biomolecules in the sample are obtained from a database. In some aspects, the compressing the dynamic range comprises a decreased first ratio relative to the second ratio. In further aspects, the decreased first ratio is at least 1.1-fold, at least 1.2-fold, at least 1.3-fold, at least 1.4-fold, at least 1.5-fold, at least 2-fold, at least 2.5-fold, at least 3-fold, at least 3.5-fold, at least 4-fold, at least 5-fold, at least 10-fold, at least 100-fold, at least 1000-fold, or at least 10,000-fold less than the second ratio.

A biomolecule of interest (e.g., a low abundance protein) may be enriched in a biomolecule corona relative to the untreated sample (e.g., a sample that is not assayed using particles). In some embodiments, a level of enrichment may be the percent increase or fold increase in concentration of the biomolecule of interest relative to the total biomolecule concentration in the biomolecule corona as compared to the untreated sample. A biomolecule of interest may be enriched in a biomolecule corona by increasing the concentration of the biomolecule of interest in the biomolecule corona as compared to the sample that has not been contacted to a particle. A biomolecule of interest may be enriched by decreasing the concentration of a high abundance biomolecule in the biomolecule corona as compared to the sample that has not been contacted to a particle. A biomolecule corona analysis assay may be used to rapidly identify low abundance biomolecules in a biological sample (e.g., a biofluid). In some embodiments, a biomolecule corona analysis may identify at least about 500 low abundance biomolecules in a biological sample in no more than about 8 hours from first contacting the biological sample with a particle. In some embodiments, a biomolecule corona analysis may identify at least about 1000 low abundance biomolecules in a biological sample in no more than about 8 hours from first contacting the biological sample with a particle. In some embodiments, a biomolecule corona analysis may identify at least about 500 low abundance biomolecules in a biological sample in no more than about 4 hours from first contacting the biological sample with a particle. In some embodiments, a biomolecule corona analysis may identify at least about 1000 low abundance biomolecules in a biological sample in no more than about 4 hours from first contacting the biological sample with a particle.

### Protein Corona Analysis in Biological Samples

The particles and methods of use thereof disclosed herein can bind a large number of proteins or protein groups in a biological sample (e.g., a biofluid). Non-limiting examples of biological samples that may be analyzed using the protein corona analysis methods described herein include biofluid samples (e.g., cerebral spinal fluid (CSF), synovial fluid (SF), urine, plasma, serum, tears, semen, whole blood, milk, nipple aspirate, ductal lavage, vaginal fluid, nasal fluid, ear fluid, gastric fluid, pancreatic fluid, trabecular fluid, lung lavage, prostatic fluid, sputum, fecal matter, bronchial lavage, fluid from swabbings, bronchial aspirants, sweat or saliva), fluidized solids (e.g., a tissue homogenate), or samples derived from cell culture. Protein corona analysis of the biomolecule corona may compress the dynamic range of the analysis compared to a total protein analysis method.

The compositions and methods disclosed herein can be used to identify various biological states in a particular biological sample. For example, a biological state can refer to an elevated or low level of a particular protein or a set of proteins. In other examples, a biological state can refer to a disease. One or more particle types can be incubated with a sample (e.g., CSF), allowing for formation of a protein corona. Said protein corona can then be analyzed by gel electrophoresis or mass spectrometry in order to identify a pattern of proteins or protein groups. Analysis of protein corona (e.g., by mass spectrometry or gel electrophoresis) may be referred to as corona analysis. The pattern of proteins or protein groups can be compared to the same methods carried out on a control sample. Upon comparison of the patterns of proteins or protein groups, it may be identified that the first sample comprises an elevated level of markers corresponding to a particular biological states. The particles and methods of use thereof, can thus be used to diagnose a particular disease state.

### Proteins Assayed

In some embodiments, the methods and compositions of the present disclosure provide identification and measurement of particular proteins in the biological samples by processing of the proteomic data via digestion of coronas formed on the surface of particles. Examples of proteins that can be identified and measured include highly abundant proteins, proteins of medium abundance, and low-abundance proteins. A low abundance protein may be present in a sample at concentrations at or below about 10 ng/mL. A high abundance protein may be present in a sample at concentrations at or above about 10 μg/mL. A protein of moderate abundance may be present in a sample at concentrations between about 10 ng/ml and about 10 μg/mL. Examples of proteins that are highly abundant proteins include albumin, IgG, and the top 14 proteins in abundance that contribute 95% of the mass in plasma. Additionally, any proteins that may be purified using a conventional depletion column may be directly detected in a sample using the particle panels disclosed herein. Examples of proteins may be any protein listed in published databases such as Keshishian et al. (Mol Cell Proteomics. 2015 September; 14 (9): 2375-93. doi: 10.1074/mcp.M114.046813. Epub 2015 Feb. 27.), Farr et al. (J Proteome Res. 2014 Jan. 3; 13 (1): 60-75. doi: 10.1021/pr4010037. Epub 2013 Dec. 6.), or Pernemalm et al. (Expert Rev Proteomics. 2014 August; 11 (4): 431-48. doi: 10.1586/14789450.2014.901157. Epub 2014 Mar. 24.).

In some embodiments, examples of proteins that can be measured and identified using the methods and compositions disclosed herein include albumin, IgG, lysozyme, CEA, HER-2/neu, bladder tumor antigen, thyroglobulin, alpha-fetoprotein, PSA, CA125, CA19.9, CA15.3, leptin, prolactin, osteopontin, IGF-II, CD98, fascin, sPigR, 14-3-3 eta, troponin I, B-type natriuretic peptide, BRCA1, c-Myc, IL-6, fibrinogen. EGFR, gastrin, PH, G-CSF, desmin. NSE, FSH, VEGF, P21, PCNA, calcitonin, PR, CA125, LH, somatostatin. S100, insulin. alpha-prolactin, ACTH, Bcl-2, ER alpha, Ki-67, p53, cathepsin D, beta catenin. VWF, CD15, k-ras, caspase 3, EPN, CD10, FAS, BRCA2. CD30L, CD30, CGA, CRP, prothrombin, CD44, APEX, transferrin, GM-CSF, E-cadherin, IL-2, Bax, IFN-gamma, beta-2-MG, TNF alpha, c-erbB-2, trypsin, cyclin D1, MG B, XBP-1, HG-1, YKL-40, S-gamma, NESP-55, netrin-1, geminin, GADD45A, CDK-6, CCL21, BrMS1, 17betaHDI, PDGFRA, Pcaf, CCL5, MMP3, claudin-4, and claudin-3. In some embodiments, other examples of proteins that can be measured and identified using the particle panels disclosed herein are any proteins or protein groups listed in the open targets database for a particular disease indication of interest.

The proteomic data of the biological sample can be identified, measured, and quantified using a number of different analytical techniques. For example, proteomic data can be analyzed using SDS-PAGE or any gel-based separation technique. Peptides and proteins can also be identified, measured, and quantified using an immunoassay, such as ELISA. Alternatively, proteomic data can be identified, measured, and quantified using mass spectrometry, high performance liquid chromatography, LC-MS/MS, Edman Degradation, immunoaffinity techniques, methods disclosed in EP3548652, WO2019083856, WO2019133892, each of which is incorporated herein by reference in its entirety, and other protein separation techniques.

In some cases, a measurement technique identifies protein groups. A measurement technique designed to detect proteins may also detect protein groups. Protein groups can refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct particle types in a particle panel, yields a high number of feature intensities.

A protein group may be a group of proteins with similar or indistinguishable mass spectrometric fingerprints. The number of protein groups identified in an assay may correlate with the number of unique proteins detected. In some cases, a protein group may comprise a set of protein isoforms. In some cases, a protein group may comprise proteins from multiple protein families. In some cases, a protein group may consist of proteins from a single protein family.

A method may also identify a biomolecule group. A biomolecule group may be a group of biomolecules which generate similar or indistinguishable signals. For example, a biomolecule group may be two biomolecules which share a retention time in a chromatographic assay, or which share a common set of mass spectrometric features in a mass spectrometry assay.

### Conditions Affecting Substrate Biomolecule Adsorbates

In some cases, the composition of biomolecules adsorbed to a substrate may be affected by solution conditions under which the substrate comes into contact with the biomolecules. Such conditions may include pH, osmolarity, salinity, solution dielectric, viscosity, temperature, surfactant concentration, and sample dilution. The composition of biomolecules adsorbed to a substrate may also be responsive to the types and concentrations of solutes present, including salts, buffers, surfactants, and other biomolecules (e.g., metabolites or nucleic acids).

The present disclosure provides a range of method and strategies for exploiting substrate (e.g., particle) surface area and surface area to mass ratios to increase profiling sensitivity, depth, and accuracy. In some aspects, the present disclosure provides a method for assaying a biological sample using a substrate, the method comprising: contacting the biological sample with the substrate to from thereon a biomolecule corona which comprises biomolecules from the biological sample, wherein the substrate has a first surface area to mass ratio; and assaying the biomolecule corona to identify the biomolecules. The method may comprise a degree of optimization in terms of substrate surface area to mass ratio. For example, in some cases, the number of different biomolecules identified is higher than the number of different biomolecules identified when the biological sample is contacted with a substrate having a second surface area to mass ratio which is different from the first surface area to mass ratio.

Surface area to mass ratio may affect the number biomolecules identified in a biomolecule corona assay. In some cases, the number of different biomolecules identified is at least 5% higher than the number of different biomolecules identified when the biological sample is contacted with the substrate having the second surface area to mass ratio. In some cases, the number of different biomolecules identified is at least 10% higher than the number of different biomolecules identified when the biological sample is contacted with the substrate having the second surface area to mass ratio. In some cases, the number of different biomolecules identified is at least 15% higher than the number of different biomolecules identified when the biological sample is contacted with the substrate having the second surface area to mass ratio. In some cases, the number of different biomolecules identified is at least 20% higher than the number of different biomolecules identified when the biological sample is contacted with the substrate having the second surface area to mass ratio. In some cases, the number of different biomolecules identified is at least 25% higher than the number of different biomolecules identified when the biological sample is contacted with the substrate having the second surface area to mass ratio. In some cases, the number of different biomolecules identified is at least 30% higher than the number of different biomolecules identified when the biological sample is contacted with the substrate having the second surface area to mass ratio. In some cases, the number of different biomolecules identified is at least 35% higher than the number of different biomolecules identified when the biological sample is contacted with the substrate having the second surface area to mass ratio. In some cases, the number of different biomolecules identified is at least 40% higher than the number of different biomolecules identified when the biological sample is contacted with the substrate having the second surface area to mass ratio. In some cases, the number of different biomolecules identified is at least 50% higher than the number of different biomolecules identified when the biological sample is contacted with the substrate having the second surface area to mass ratio. In some cases, the number of different biomolecules identified is at least 75% higher than the number of different biomolecules identified when the biological sample is contacted with the substrate having the second surface area to mass ratio. In some cases, the number of different biomolecules identified is at least twice that of the number of different biomolecules identified when the biological sample is contacted with the substrate having the second surface area to mass ratio.

Substrate surface area to mass ratio can intimately affect the composition and time course for biomolecule corona formation. Small variations in substrate surface area to mass ratios can impart pronounced effects on substrate behavior and properties. Principally among these, higher surface area to mass ratios often lend to greater substrate solubilities and hydrophilicities, thus modifying their biomolecule affinities. Substrate surface area to mass ratios often also affect substrate diffusion, with lower surface area to mass ratios biasing substrates for faster diffusion and, in some cases, faster kinetics for biomolecule corona formation. In the above outlined method, the second surface area to mass ratio may be greater than the first surface area to mass ratio. Alternatively, the second surface area to mass ratio may be lower than the first surface area to mass ratio. In some cases, the substrate having the first surface area to mass ratio has a greater surface area than the substrate having the second surface area to mass ratio. For example, the substrate having the first surface area to mass ratio may have at least 10% greater, at least 25% greater, at least 50% greater, at least 100% greater, at least 150% greater, at least 200% greater, at least 350% greater, at least 500% greater, at least 1000% greater, at least 5000% greater, or at least 10000% greater surface area than the substrate having the second surface area to mass ratio. In other cases, the substrate having the first surface area to mass ratio has a lower surface area than the substrate having the second surface area to mass ratio.

In some cases, the surface area to mass ratio difference between the substrate having the first surface area to mass ratio and the substrate having the second surface area to mass ratio is primarily to due at least in part to morphology. For example, the substrate having the first surface area to mass ratio and the substrate having the second surface area to mass ratio may have densities differing by at most 5%, at most 10%, at most 15%, at most 20%, at most 25%, at most 30%, at most 40%, at most 50%, at most 60%, at most 70%, at most 80%, or at most 90%. Alternatively, the surface area to mass ratio difference between the substrate having the first surface area to mass ratio and the substrate having the second surface area to mass ratio may comprise a density contribution. In some cases, the substrate having the first surface area to mass ratio and the substrate having the second surface area to mass ratio may have densities differing by at least 5%, at least 10%, at least 15%, at least 20%, at least 25%, at least 30%, at least 40%, at least 50%, at least 60%, at least 70%, at least 80%, or at least 90%. For example, the substrate having the first surface area to mass ratio may comprise a low density styrene particle with an average density of around 1 g/cm³, and the substrate having the second surface area to mass ratio may comprise a relatively high density gold alloy particle with an average density of around 16 g/cm³. In some cases, the substrate having the first surface area to mass ratio and the substrate having the second surface area to mass ratio substrate are particles having diameters (e.g., average diameters) differing from each other by at most 5%, at most 10%, at most 15%, at most 20%, at most 25%, at most 30%, at most 35%, at most 40%, at most 50%, at most 60%, or at most 80%. In some cases, the substrate having the first surface area to mass ratio and the substrate having the second surface area to mass ratio substrate are particles having diameters (e.g., average diameters) differing from each other by at least 5%, at least 10%, at least 15%, at least 20%, at least 25%, at least 30%, at least 35%, at least 40%, at least 50%, at least 60%, or at least 80%.

The substrates may comprise differences in morphologies. In some cases, both substrates comprise particles. For example, the substrate having the first surface area to mass ratio and the substrate having the second surface area to mass ratio may both be nanoparticles. In other cases, the substrate having the first surface area to mass ratio and the substrate having the second surface area to mass ratio are both microparticles. Alternatively, one substrate may be a nanoparticle and the other substrate may be a microparticle. In some cases, the substrate having the first surface area to mass ratio and the substrate having the second surface area to mass ratio may both be the same type of particle. For example, the substrate having the first surface area to mass ratio may comprise an 80 nm carboxyl functionalized styrene particle, and the substrate having the second surface area to mass ratio may comprise a 200 nm carboxyl functionalized styrene particle. In some cases, one or both substrates comprise a plurality of particles. In some cases, the plurality of particles comprises a nanoparticle and a microparticle. In some cases, one or both substrates comprises a nanorod, a nanowire, a nanotube, an extended surface (such as a glass slide), a nanowell, a nanotrench, an imprinted polymer, a polymer matrix, a gel (e.g., a hydrogel), a half-particle, or any combination thereof. In some cases, a substrate is coupled to a surface, such as a glass slide or a surface of a fluidic chamber.

In some cases, the substrate having the first surface area to mass ratio forms a colloid upon contacting the biological sample. Conversion of a liquid biological sample to a colloidal suspension can alter biomolecule solubilities, and can thereby affect biomolecule affinities for particle binding. Accordingly, a particle may generate a different biomolecule corona when provided as a colloid, rather than as a dilute suspension. In some cases, the substrate having the second surface area to mass ratio does not form a colloid upon contact with the biological sample. In other cases, the substrate having the second surface area to mass ratio forms a colloid upon contact with the biological sample.

In some cases, the method comprises assaying the biomolecule corona prior to the biomolecule corona achieving equilibrium. In such cases, the composition of the biomolecule corona subjected to the assaying and the composition of the biomolecule corona subsequent to said achieving said equilibrium share at most 95%, at most 90%, at most 85%, at most 80%, at most 75%, at most 70%, at most 65%, at most 60%, at most 50%, at most 40%, or at most 30% of proteins in common.

Profiling sensitivity, depth, and accuracy may also comprise a dependence on substrate homogeneity. As biomolecule corona composition can be sensitive to substrate surface area, mass, and surface area to mass ratio (e.g., particle diameter), substrate homogeneity can impact biomolecule composition and mass yield. A substrate (e.g., a nanoparticle) comprising a relatively high polydispersity index, and therefore a relatively high degree of size or mass heterogeneity, may collect a greater number of biomolecules from a sample. A substrate comprising a relatively low polydispersity index, and thus comprising a degree of size or mass uniformity, may exhibit a higher degree of biomolecule corona uniformity across replicates. In the methods outlined above, the substrate having the first surface area to mass ratio and the substrate having the second surface area to mass ratio may both comprise polydispersity indices of at least 0.1, at least 0.2, at least 0.3, at least 0.4, at least 0.5, at least 0.6, at least 0.8, at least 1, at least 1.2, at least 1.4, at least 1.6, at least 1.8, or at least 2. The substrate having the first surface area to mass ratio and the substrate having the second surface area to mass ratio may both comprise polydispersity indices of at most 2, at most 1.8, at most 1.6, at most 1.4, at most 1.2, at most 1, at most 0.8, at most 0.6, at most 0.5, at most 0.4, at most 0.3, at most 0.2, or at most 0.1. The substrate having the first surface area to mass ratio and the substrate having the second surface area to mass ratio may comprise different polydispersity indices. The substrate having the first surface area to mass ratio and the substrate having the second surface area to mass ratio may comprise polydispersity indices differing by at least 0.05, at least 0.1, at least 0.2, at least 0.3, at least 0.4, at least 0.5, at least 0.6, at least 0.8, at least 1, at least 1.2, at least 1.4, at least 1.6, at least 1.8, or at least 2. For example, the substrate having the first surface area to mass ratio and the substrate having the second surface area to mass ratio may both comprise carboxyl functionalized styrene particles with 120 nm average diameters, but different size standard deviations (e.g., 30 nm and 4 nm). Alternatively, the substrate having the first surface area to mass ratio and the substrate having the second surface area to mass ratio may comprise polydispersity indices differing by at most 2, at most 1.8, at most 1.6, at most 1.4, at most 1.2, at most 1, at most 0.8, at most 0.6, at most 0.5, at most 0.4, at most 0.3, at most 0.2, or at most 0.1.

Alternatively or in addition to, the number of different biomolecules identified is higher than the number of different biomolecules identified when the biological sample is assayed with an amount of the substrate that is 10% or more greater than the amount of the substrate used for the contacting. For example, a method may comprise contacting the biological sample with the substrate to form thereon a biomolecule corona which comprises biomolecules from the biological sample, wherein the substrate has a surface area to mass ratio of from 1 to 6000 cm²/mg; and assaying the biomolecule corona to identify the biomolecules, wherein the number of different biomolecules identified is higher than the number of different biomolecules identified when the biological sample is assayed with an amount of the substrate that is 10% or more greater than the amount of the substrate used for said contacting.

The present disclosure provides a range of strategies for modifying substrate concentration to enhance biomolecule detection. Various aspects of the present disclosure provide a method of assaying a biological sample using a substrate, the method comprising: contacting the biological sample with the substrate to form thereon a biomolecule corona which comprises biomolecules from the biological sample, wherein the substrate has a surface area to mass ratio of from 1 to 6000 cm²/mg; and assaying the biomolecule corona to identify the biomolecules, wherein the number of different biomolecules identified is higher than the number of different biomolecules identified when the biological sample is assayed with an amount of the substrate that is 5% or more greater than the amount of the substrate contacted to the sample. In some cases, the number of different biomolecules identified is higher than the number of different biomolecules identified when the biological sample is assayed with an amount of the substrate that is 10% or more greater than the amount of the substrate contacted to the sample. In some cases, the number of different biomolecules identified is higher than the number of different biomolecules identified when the biological sample is assayed with an amount of the substrate that is 20% or more greater than the amount of the substrate contacted to the sample. In some cases, the number of different biomolecules identified is higher than the number of different biomolecules identified when the biological sample is assayed with an amount of the substrate that is 30% or more greater than the amount of the substrate contacted to the sample. In some cases, the number of different biomolecules identified is higher than the number of different biomolecules identified when the biological sample is assayed with an amount of the substrate that is 50% or more greater than the amount of the substrate contacted to the sample. In some cases, the number of different biomolecules identified is higher than the number of different biomolecules identified when the biological sample is assayed with an amount of the substrate that is 100% or more greater than the amount of the substrate contacted to the sample.

In some cases, the identified biomolecules span at least 0.5 order of magnitude greater in concentration than biomolecules identified when the biological sample is assayed with an amount of the substrate that is at least 5% or more greater. In some cases, the identified biomolecules span at least 1 order of magnitude greater in concentration than biomolecules identified when said biological sample is assayed with an amount of the substrate that is at least 5% or more greater. In some cases, the identified biomolecules span at least 1.5 order of magnitude greater in concentration than biomolecules identified when said biological sample is assayed with an amount of the substrate that is at least 5% or more greater. In some cases, the identified biomolecules span at least 2 order of magnitude greater in concentration than biomolecules identified when said biological sample is assayed with an amount of the substrate that is at least 5% or more greater. In some cases, the identified biomolecules span at least 3 order of magnitude greater in concentration than biomolecules identified when said biological sample is assayed with an amount of the substrate that is at least 5% or more greater.

In some cases, the quantity of substrate contacted to the biomolecule sample diminishes the dynamic range of biomolecules assayed. Such dynamic range contraction can increase the intensity of signals for low abundance biomolecules, for example by diminishing signal contributions from high abundance proteins. In some cases, the identified biomolecules span at least 0.25 order of magnitude less in concentration than biomolecules identified when said biological sample is assayed with an amount of the substrate that is at least 5% or more greater. In some cases, the identified biomolecules span at least 0.5 order of magnitude less in concentration than biomolecules identified when said biological sample is assayed with an amount of the substrate that is at least 5% or more greater. In some cases, the identified biomolecules span at least 0.75 order of magnitude less in concentration than biomolecules identified when said biological sample is assayed with an amount of the substrate that is at least 5% or more greater. In some cases, the identified biomolecules span at least 1 order of magnitude less in concentration than biomolecules identified when said biological sample is assayed with an amount of the substrate that is at least 5% or more greater. In some cases, the identified biomolecules span at least 1.5 order of magnitude less in concentration than biomolecules identified when said biological sample is assayed with an amount of the substrate that is at least 5% or more greater.

Substrate quantity may also be optimized to diminish signals from high abundance biomolecules from a biological sample. For example, low abundance plasma biomolecule detection is often hampered by intense signals from high abundance plasma proteins, such as albumin and globulins. The amount of substrate used for an assay may diminish albumin and globulin collection, thereby making it possible to resolve low abundance proteins, such as cytokines. In some cases, the biological sample comprises plasma, and the identified biomolecules comprise a lower proportion of albumin and globulins that biomolecules identified when the biological sample is assayed with an amount of the substrate that is 5% or more greater than the amount of the substrate used for the assay. In some cases, the biological sample comprises plasma, and the identified biomolecules comprise a lower proportion of albumin and globulins that biomolecules identified when the biological sample is assayed with an amount of the substrate that is 10% or more greater than the amount of the substrate used for the assay. In some cases, the biological sample comprises plasma, and the identified biomolecules comprise a lower proportion of albumin and globulins that biomolecules identified when the biological sample is assayed with an amount of the substrate that is 20% or more greater than the amount of the substrate used for the assay. In some cases, the biological sample comprises plasma, and the identified biomolecules comprise a lower proportion of albumin and globulins that biomolecules identified when the biological sample is assayed with an amount of the substrate that is 30% or more greater than the amount of the substrate used for the assay. In some cases, the biological sample comprises plasma, and the identified biomolecules comprise a lower proportion of albumin and globulins that biomolecules identified when the biological sample is assayed with an amount of the substrate that is 50% or more greater than the amount of the substrate used for the assay. In some cases, the biological sample comprises plasma, and the identified biomolecules comprise a lower proportion of albumin and globulins that biomolecules identified when the biological sample is assayed with an amount of the substrate that is 75% or more greater than the amount of the substrate used for the assay. In some cases, the biological sample comprises plasma, and the identified biomolecules comprise a lower proportion of albumin and globulins that biomolecules identified when the biological sample is assayed with an amount of the substrate that is 100% or more greater than the amount of the substrate used for the assay.

In some cases, the substrate has a density of between about 0.05 grams and about 5 grams per cubic centimeter. In some cases, the substrate has a density of between about 0.1 grams and about 4 grams per cubic centimeter. In some cases, the substrate has a density of between about 0.2 grams and about 3 grams per cubic centimeter. In some cases, the substrate has a density of between about 0.2 grams and about 0.5 grams per cubic centimeter. In some cases, the substrate has a density of between about 0.4 grams and about 1 grams per cubic centimeter. In some cases, the substrate has a density of between about 0.8 grams and about 2 grams per cubic centimeter. In some cases, the substrate has a density of between about 1.2 grams and about 3 grams per cubic centimeter. In some cases, the substrate has a density of between about 1.5 grams and about 5 grams per cubic centimeter. In some cases, the substrate has a density of at least 2 grams per cubic centimeter. In some cases, the substrate has a density of between 0.05 and 15 grams per cubic centimeter. In some cases, the substrate has a density of at least 0.05 grams per cubic centimeter. In some cases, the substrate has a density of at least 0.1 grams per cubic centimeter. In some cases, the substrate has a density of at least 0.2 grams per cubic centimeter. In some cases, the substrate has a density of at least 0.4 grams per cubic centimeter. In some cases, the substrate has a density of at least 0.8 grams per cubic centimeter. In some cases, the substrate has a density of at least 1.2 grams per cubic centimeter. In some cases, the substrate has a density of at least 1.5 grams per cubic centimeter. In some cases, the substrate has a density of at least 2 grams per cubic centimeter. In some cases, the substrate has a density of at least 3 grams per cubic centimeter. In some cases, the substrate has a density of at least 5 grams per cubic centimeter. In some cases, the substrate has a density of at least 8 grams per cubic centimeter. In some cases, the substrate has a density of at least 10 grams per cubic centimeter. In some cases, the substrate has a density of at least 12 grams per cubic centimeter. In some cases, the substrate has a density of at least 15 grams per cubic centimeter. In some cases, the substrate has a density of at most 0.05 grams per cubic centimeter. In some cases, the substrate has a density of at most 0.1 grams per cubic centimeter. In some cases, the substrate has a density of at most 0.2 grams per cubic centimeter. In some cases, the substrate has a density of at most 0.4 grams per cubic centimeter. In some cases, the substrate has a density of at most 0.8 grams per cubic centimeter. In some cases, the substrate has a density of at most 1.2 grams per cubic centimeter. In some cases, the substrate has a density of at most 1.5 grams per cubic centimeter. In some cases, the substrate has a density of at most 2 grams per cubic centimeter. In some cases, the substrate has a density of at most 3 grams per cubic centimeter. In some cases, the substrate has a density of at most 5 grams per cubic centimeter. In some cases, the substrate has a density of at most 8 grams per cubic centimeter. In some cases, the substrate has a density of at most 10 grams per cubic centimeter. In some cases, the substrate has a density of at most 12 grams per cubic centimeter. In some cases, the substrate has a density of at most 15 grams per cubic centimeter.

In addition to biomolecule corona composition, biomolecule corona mass can be sensitive to a range of factors including substrate type, surface area to mass ratio, sample conditions, and sample type. In some cases, the biomolecule corona comprises at least 0.01 micrograms (μg) biomolecules per milligram (mg) substrate. In some cases, the biomolecule corona comprises at least 0.1 micrograms (μg) biomolecules per milligram (mg) substrate. In some cases, the biomolecule corona comprises at least 1 microgram (μg) biomolecules per milligram (mg) substrate. In some cases, the biomolecule corona comprises at least 10 micrograms (μg) biomolecules per milligram (mg) substrate. In some cases, the biomolecule corona comprises at least 100 micrograms (μg) biomolecules per milligram (mg) substrate. In some cases, the biomolecule corona comprises at most 100 micrograms (μg) biomolecules per milligram (mg) substrate. In some cases, the biomolecule corona comprises at most 10 micrograms (μg) biomolecules per milligram (mg) substrate. In some cases, the biomolecule corona comprises at most 1 microgram (μg) biomolecules per milligram (mg) substrate. In some cases, the biomolecule corona comprises at most 0.1 micrograms (μg) biomolecules per milligram (mg) substrate. In some cases, the biomolecule corona comprises at most 0.01 micrograms (μg) biomolecules per milligram (mg) substrate. In some cases, the biomolecule corona comprises at least 0.01 μg biomolecules per 100 square centimeter (cm²) substrate. In some cases, the biomolecule corona comprises at least 0.1 μg biomolecules per 100 square centimeter (cm²) substrate. In some cases, the biomolecule corona comprises at least 1 μg biomolecules per 100 square centimeter (cm²) substrate. In some cases, the biomolecule corona comprises at least 10 μg biomolecules per 100 square centimeter (cm²) substrate. In some cases, the biomolecule corona comprises at least 100 μg biomolecules per 100 square centimeter (cm²) substrate. In some cases, the biomolecule corona comprises at least 1 mg biomolecules per 100 square centimeter (cm²) substrate. In some cases, the biomolecule corona comprises at most 1 mg biomolecules per 100 square centimeter (cm²) substrate. In some cases, the biomolecule corona comprises at most 100 μg biomolecules per 100 square centimeter (cm²) substrate. In some cases, the biomolecule corona comprises at most 10 μg biomolecules per 100 square centimeter (cm²) substrate. In some cases, the biomolecule corona comprises at most 1 μg biomolecules per 100 square centimeter (cm²) substrate. In some cases, the biomolecule corona comprises at most 0.1 μg biomolecules per 100 square centimeter (cm²) substrate. In some cases, the biomolecule corona comprises at most 0.01 μg biomolecules per 100 square centimeter (cm²) substrate.

The amount and types of biomolecules adsorbed by substrate in a sample can depend on the ratio between aggregate substrate surface area (e.g., the combined surface areas of a plurality of particles in a solution) and sample volume. In some cases, a change in the aggregate substrate surface area to sample volume ratio can change the total amount (e.g., total mass) of biomolecules adsorbed to the substrate in a solution by 1%, 2%, 3%, 4%, 5%, 6%, 7%, 8%, 9%, 10%, 12%, 15%, 20%, 25%, 30%, 35%, 40%, 45%, 50% or more. In some cases, a change in the aggregate substrate surface area to sample volume ratio can change the composition (e.g., the collective types) of biomolecules adsorbed to the substrate in a solution by 1%, 2%, 3%, 4%, 5%, 6%, 7%, 8%, 9%, 10%, 12%, 15%, 20%, 25%, 30%, 35%, 40%, 45%, 50%, 60%, or more.

In some cases, the change in ratio between the aggregate substrate surface area to sample volume required to impart such effects is less than 80%, 70%, 60%, 50%, 40%, 30%, 25%, 20%, 15%, 10%, 5%, 4%, 3%, 2%, or 1%.

In some cases, the ratio of substrate surface area to substrate mass per unit volume of the sample affects the amount and composition of biomolecules that adsorb to the substrate. In some cases, the ratio of substrate surface area to substrate mass ratio to a volume of the sample is between 20 to 5000 cm² mg⁻¹ ml⁻¹. In some cases, the ratio of substrate surface area to substrate mass ratio to a volume of the sample is between 20 to 1000 cm² mg⁻¹ ml⁻¹, 30 to 1200 cm² mg⁻¹ ml-1, 40 to 1400 cm² mg⁻¹ ml⁻¹, 50 to 1600 cm² mg⁻¹ ml⁻¹, 60 to 1800 cm² mg⁻¹ ml⁻¹, 80 to 2000 cm² mg⁻¹ ml⁻¹, 100 to 2400 cm² mg⁻¹ ml⁻¹, 120 to 2700 cm² mg⁻¹ ml⁻¹, 150 to 3000 cm² mg⁻¹ ml⁻¹, 200 to 4000 cm² mg⁻¹ ml⁻¹, 300 to 5000 cm² mg⁻¹ ml⁻¹, 400 to 6000 cm² mg⁻¹ ml⁻¹, 500 to 8000 cm² mg⁻¹ ml⁻¹, 800 to 10000 cm² mg⁻¹ ml⁻¹, 20 to 1000 cm² mg⁻¹ ml⁻¹, 50 to 3500 cm² mg⁻¹ ml⁻¹ or 100 to 3000 cm² mg 1 ml⁻¹. In some cases, the ratio of substrate surface area to substrate mass ratio to a volume of the sample is between 200 to 1800 cm² mg⁻¹ ml⁻¹.

In some cases, decreasing the concentration, aggregate surface area, or aggregate mass of particles contacted to a sample increases the number of types of biomolecules which adsorb to the particle surfaces. In some cases, decreasing the concentration, aggregate surface area, or aggregate mass of particles contacted to a sample increases the number of types of proteins which adsorb to the particle surfaces. For example, halving a concentration of particles/or surface area contacted to a sample may increase the number of types of proteins collected on the particle surfaces by at least 5%, at least 10%, at least 15%, at least 20%, at least 25%, at least 30%, at least 35%, at least 40%, at least 45%, or at least 50%.

Different concentrations of a particle may generate distinct biomolecule coronas upon contact with a sample. In some cases, contacting separate portions of a sample with different concentrations of a particle increases the number of types of biomolecules collected from the sample (as compared to contacting a single portion of the sample with a single particle concentration). Accordingly, a method consistent with the present disclosure may comprise contacting multiple portions of a sample with at least 2 concentrations of a particle, at least 3 concentrations of a particle, at least 4 concentrations of a particle, at least 5 concentrations of a particle, at least 6 concentrations of a particle, at least 7 concentrations of a particle at least 8 concentrations of a particle, at least 10 concentrations of a particle, or at least 12 concentrations of a particle. The types of biomolecules in biomolecule coronas of two samples contacted with different concentrations of the same particle can differ by at least 2%, at least 4%, at least 6%, at least 8%, at least 10%, at least 15%, at least 20%, at least 25%, or at least 30%.

Different particle concentrations may generate biomolecule coronas with different dynamic ranges. In some cases, a plurality of biomolecule coronas generated with a plurality of different particle concentrations comprise dynamic ranges differing by at least 0.25, at least 0.5, at least 0.75, at least 1, at least 1.5, at least 2, or at least 2.5. In some cases, a plurality of biomolecule coronas generated with a plurality of different particle concentrations comprise mean biomolecule concentrations (e.g., defined as the concentrations of the biomolecules in the sample from which the biomolecule corona was derived) by at least 0.25 orders of magnitude, at least 0.5 orders of magnitude, at least 0.75 orders of magnitude, at least 1 order of magnitude, at least 1.5 orders of magnitude, at least 2 orders of magnitude, or at least 2.5 orders of magnitude in concentration.

In some cases, lower particle concentrations generate biomolecule coronas with higher average masses. Two samples contacted with different concentrations of the same particle may generate biomolecule coronas with masses differing by at least 2%, at least 4%, at least 6%, at least 8%, at least 10%, at least 12%, at least 15%, or at least 20%.

Particle concentration can also affect the rate of biomolecule corona formation. The mass and composition of a biomolecule corona may exhibit dynamic, time-dependent profiles. Changing the concentration of particles contacted to a sample may not only affect the types and amounts of biomolecules adsorbed to the particles, but may also change the rate at which equilibrium is reestablished within the sample. In some cases, two portions of a sample contacted with different concentrations of a particle reestablish chemical equilibrium at different rates. In some cases, the biomolecule coronas of two portions of a sample contacted with different concentrations of a particle become less similar as they approach equilibrium. In some cases, the biomolecule coronas of two portions of a sample contacted with different concentrations of a particle become more similar as they approach equilibrium. A method of the present disclosure may exploit this time dependence. For example, a biomolecule corona may be collected from a sample and assayed (e.g., biomolecules of the biomolecule corona may be identified by mass spectrometry) prior to reaching equilibrium with the sample. Conversely, a method of the present disclosure may comprise collecting a biomolecule corona once a system has achieved equilibrium (e.g., wherein a relative rate of change in biomolecule corona composition is less than 2%, less than 1%, less than 0.5%, less than 0.2%, or less than 0.1% of its maximum value). A method may comprise contacting a sample with a particle for at least 1 minute, at least 2 minutes, at least 3 minutes, at least 4 minutes, at least 5 minutes, at least 6 minutes, at least 8 minutes, at least 10 minutes, at least 12 minutes, at least 15 minutes, at least 20 minutes, at least 30 minutes, at least 40 minutes, at least 1 hour, at least 1.5 hours, at least 2 hours, at least 3 hours, at least 4 hours, at least 5 hours, at least 6 hours, at least 8 hours, at least 12 hours, at least 16 hours, at least 24 hours, at least 36 hours, at least 48 hours, or at least 72 hours. A method may comprise contacting a sample with a particle for at most 72 hours, at most 48 hours, at most 36 hours, at most 24 hours, at most 16 hours, at most 12 hours, at most 8 hours, at most 6 hours, at most 5 hours, at most 4 hours, at most 3 hours, at most 2 hours, at most 1 hour, at most 40 minutes, at most 30 minutes, at most 20 minutes, at most 15 minutes, at most 12 minutes, at most 10 minutes, at most 8 minutes, at most 6 minutes, at most 5 minutes, at most 4 minutes, at most 3 minutes, at most 2 minutes, or at most 1 minute. In some cases, two portions of a sample are contacted to particles for different lengths of time. For example, a first portion of a sample may be contacted to a particle for less time than is needed to reach equilibrium, and a second portion of the sample may be contacted to a particle for a sufficient length of time to reach equilibrium.

Very large and very small substrates (e.g., small particles) can have correspondingly low ratios of substrate surface area to substrate mass ratio to a volume or correspondingly high ratios of substrate surface area to substrate mass ratio to a volume, respectively. In an example, a solution comprising substrates such as 600 nm up to 1.2 μm diameter particles can have a ratio of substrate surface area to substrate mass ratio to a volume of the sample between 1 to 100 cm² mg⁻¹ ml⁻¹. In some cases, a solution comprising substrates with diameters of 50 nm or less can have a ratio of substrate surface area to substrate mass ratio to a volume of the sample between 10000 to 100000 cm² mg⁻¹ ml⁻¹.

In some cases, adjusting the ratio of substrate surface area to substrate mass per unit volume of the sample by a minor amount (e.g., 5 or 10%) can change the amount or composition of biomolecules adsorbed to the substrate by 5%, 10%, 20%, 30%, 40%, 50%, 60% or more relative to the original conditions.

Changing the amount of substrate in a sample (e.g., a solution comprising plasma) can change the number of types of biomolecules that adsorb to the substrate. This can affect the number of biomolecules identified in an assay. In some cases, using 90% or less of the amount of a substrate (e.g., diminishing the amount of substrate used by 10% or more) can increase the number of biomolecules identified in an assay by at least 1.04, 1.1, 1.2, 1.5, 2, 5, 10, 50, or 100 times relative to the number of biomolecules that would be identified using the original amount of substrate.

### Computer Control Systems

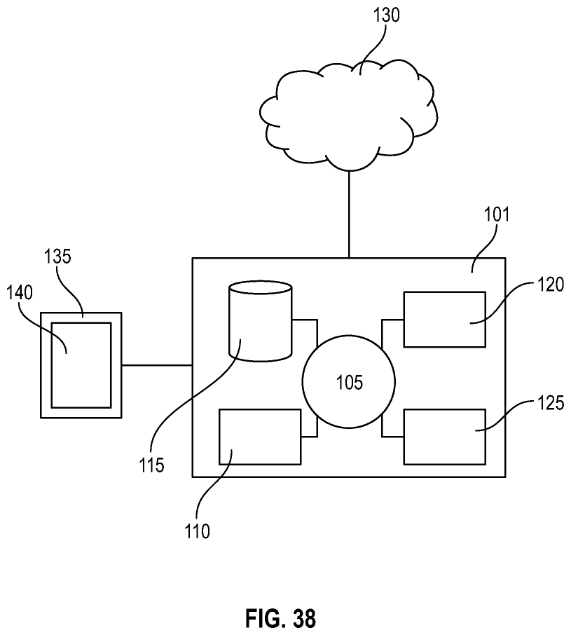

The present disclosure provides computer control systems that are programmed to implement methods of the disclosure. FIG. 38 shows a computer system that is programmed or otherwise configured to implement methods provided herein. The computer system 101 can regulate various aspects of the assays disclosed herein, which are capable of being automated (e.g., movement of any of the reagents disclosed herein on a substrate, conducting serial dilution of a particle concentration, directing a biological sample or a portion thereof into contact with one or more particle-containing solutions). The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example a readout of the proteins identified using the methods disclosed herein. Examples of UI's include, without limitation, a graphical user interface (GUI) and web-based user interface. Methods and systems of the present disclosure can be implemented by way of one or more algorithms. An algorithm can be implemented by way of software upon execution by the central processing unit 105.

Determination, analysis or statistical classification is done by methods known in the art, including, but not limited to, for example, a wide variety of supervised and unsupervised data analysis and clustering approaches such as hierarchical cluster analysis (HCA), Partial least squares Discriminant Analysis (PLSDA), machine learning (also known as random forest), logistic regression, decision trees, support vector machine (SVM), k-nearest neighbors, naive bayes, linear regression, polynomial regression, SVM for regression, K-means clustering, and hidden Markov models, among others. The computer system can perform various aspects of analyzing the protein sets or protein corona of the present disclosure, such as, for example, determining a concentration or abundance of a biomolecule or biomolecule group in a sample from data associated with the biomolecule or biomolecule group from a multiple particle concentration assay. For example, a system consistent with the present disclosure may comprise computer memory comprising data comprising information of biomolecules or biomolecule groups corresponding to a plurality of different biomolecule coronas, wherein the plurality of different biomolecule coronas is formed upon contacting a biological sample with a plurality of particle-containing solutions each having a different particle concentration; and a computer in communication with the computer memory, wherein the computer comprises a computer processor and computer readable medium comprising machine-executable code that, upon execution by the computer processor, implements a method comprising: receiving the data from the computer memory; and determining, in the absence of using information of a reference biomolecule external to the biological sample, a concentration or an amount of a biomolecule or biomolecule group in the biological sample, based on at least partially on the data. The data may comprise mass spectrometric signals associated with biomolecules or said biomolecule groups. Concentration determination may comprise comparing a plurality of signal intensities (e.g., mass spectrometric signal intensities) associated with at least a subset of said plurality of different biomolecule coronas. Concentration determination may comprise identifying a relationship between said plurality of signal intensities and particle concentrations of said plurality of particle-containing solutions. Concentration determination may comprise a comparison of signal intensities associated with the biomolecule or biomolecule group for which a concentration is determined and the intensity of a signal associated with another biomolecule or biomolecule group from the biological sample. Concentration determination may comprise computationally modeling (e.g., performing least squares fitting on) the data. Concentration determination may comprise comparing biomolecule corona data against reference data. For example, concentration determination may comprise comparing a slope of a linear regression model (e.g., particle concentration versus signal intensity or principle component analysis vectors) against a plurality of slopes from reference data sets comprising different quantities of the biomolecule or biomolecule group for which the concentration is being determined.

The computer system can be used to develop classifiers to identify trends associated with biomolecules or biomolecule groups across multiple data sets, and to extrapolate or interpolate from these trends to identify characteristics of the biomolecules (e.g., post-translational modifications or isomeric states) or samples from which they derive (e.g., sample pH or glucose concentration). Data collected from the presently disclosed sensor array can be used to train a machine learning algorithm, specifically an algorithm that receives biomolecule corona measurements and outputs biomolecule or biomolecule group type or concentration, or biomolecule or biomolecule group chemical state or sample conditions. Before training the algorithm, raw data from the array can be first denoised to reduce variability in individual variables.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FPgrowth algorithm; Hierarchical clustering, such as Singlelinkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory. A computer system may be adapted to implement a method described herein. The system includes a central computer server that is programmed to implement the methods described herein. The server includes a central processing unit (CPU, also “processor”) which can be a single core processor, a multi core processor, or plurality of processors for parallel processing. The server also includes memory (e.g., random access memory, read-only memory, flash memory); electronic storage unit (e.g. hard disk); communications interface (e.g., network adaptor) for communicating with one or more other systems; and peripheral devices which may include cache, other memory, data storage, and/or electronic display adaptors. The memory, storage unit, interface, and peripheral devices are in communication with the processor through a communications bus (solid lines), such as a motherboard. The storage unit can be a data storage unit for storing data. The server is operatively coupled to a computer network (“network”) with the aid of the communications interface. The network can be the Internet, an intranet and/or an extranet, an intranet and/or extranet that is in communication with the Internet, a telecommunication or data network. The network in some cases, with the aid of the server, can implement a peer-to-peer network, which may enable devices coupled to the server to behave as a client or a server.

The storage unit can store files, such as subject reports, and/or communications with the data about individuals, or any aspect of data associated with the present disclosure.

The computer server can communicate with one or more remote computer systems through the network. The one or more remote computer systems may be, for example, personal computers, laptops, tablets, telephones, Smart phones, or personal digital assistants.

In some applications the computer system includes a single server. In other situations, the system includes multiple servers in communication with one another through an intranet, extranet and/or the internet.

The server can be adapted to store measurement data or a database as provided herein, patient information from the subject, such as, for example, medical history, family history, demographic data and/or other clinical or personal information of potential relevance to a particular application. Such information can be stored on the storage unit or the server and such data can be transmitted through a network.

Methods as described herein can be implemented by way of machine (or computer processor) executable code (or software) stored on an electronic storage location of the server, such as, for example, on the memory, or electronic storage unit. During use, the code can be executed by the processor. In some cases, the code can be retrieved from the storage unit and stored on the memory for ready access by the processor. In some situations, the electronic storage unit can be precluded, and machine-executable instructions are stored on memory. Alternatively, the code can be executed on a second computer system.

Aspects of the systems and methods provided herein, such as the server, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

The computer systems described herein may comprise computer-executable code for performing any of the algorithms or algorithms-based methods described herein. In some applications the algorithms described herein will make use of a memory unit that is comprised of at least one database.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### Classification of Protein Corona Using Machine Learning

The method of determining a set of biomolecules associated with the disease or disorder and/or disease state include the analysis of the corona of the at least two samples. This determination, analysis or statistical classification is done by methods known in the art, including, but not limited to, for example, a wide variety of supervised and unsupervised data analysis, machine learning, deep learning, and clustering approaches including hierarchical cluster analysis (HCA), Partial least squares Discriminant Analysis (PLS-DA), random forest, logistic regression, decision trees, support vector machine (SVM), k-nearest neighbors, naive bayes, linear regression, polynomial regression, SVM for regression, K-means clustering, and hidden Markov models, among others. In other words, the biomolecules in the corona of each sample are compared/analyzed with each other to determine with statistical significance what patterns are common between the individual corona to determine a set of biomolecules that is associated with the disease or disorder or disease state.

Generally, machine learning algorithms are used to construct models that accurately assign class labels to examples based on the input features that describe the example. In some case it may be advantageous to employ machine learning and/or deep learning approaches for the methods described herein. For example, machine learning can be used to associate assay conditions or particle characteristics with the biomolecule corona and adsorbed or associated biomolecules. In other cases, machine learning can be used to associate the biomolecule corona with various disease states (e.g. no disease, precursor to a disease, having early or late stage of the disease, etc.). For example, in some cases, one or more machine learning algorithms are employed in connection with a method of the invention to analyze data detected and obtained by the biomolecule corona and sets of biomolecules derived therefrom. For example, in one embodiment, machine learning can be coupled with the sensor array described herein to determine not only if a subject (i) is at a risk for developing a disease, (ii) has a preclinical stage of a disease, or (iii) the disease. In some embodiments, machine learning can be coupled with the sensor array described herein to determine a prognosis of a disease. In some embodiments, machine learning can be coupled with the sensor array described herein to distinguish between subtypes of a disease. In yet other embodiments, machine learning can be coupled with the sensor array described herein to determine no disease in a subject.

### Mass Spectrometry Workflow Aspects of Dilutions.

Buffer and concentration aspects affecting proteolysis. Components of the workflow can include pH between 7-8, <4 inhibits trypsin, some buffer components can reduce Trypsin activity, too low/too high concentration of chaotropic and detergents reduces efficiency, very low peptide concentration can cause loss by adsorption (pipetting/tube), digestion temperature either RT (urea) or 37° C. most other buffers. Buffer systems can include 10⁻⁵⁰ mM HEPES, TRIS, or ammonium bicarbonate+2-8 M Urea/Thiourea, 0.5-6 M GuHCl, 4% SDS (with precipitation), or 1-4% SDC.

Buffer and concentration aspects affecting peptide clean-up. Components of the workflow can include solid Phase extraction (SPE) needs to: concentrate peptides, remove salt, Filter debris/precipitates and proteins; gets compromised by polymers like PEG and SDS; peptides need to be in with the binding capacity of the matrix and acidified (c18) otherwise loss of less hydrophobic species; buffer components can leak to next samples; can be an issues if NPs are scrambled.

Buffer and concentration aspects affecting LC-MS/MS. Components of the workflow can include high amounts of Ion suppression agents (detergents/polymer/TFA) including non peptide charge carriers, increased surface tension, nonvolatile substances; interfering signal including polymers, metal (adducts); modification inducing/crosslinking material; nonvolatile and corrosive agents; injection material depends on workflow (ng(TIMs)−ug (Sciex)); charge state coalescence (e.g. can desired or problematic).

### Biomolecule Quantification

Biomolecule corona composition can exhibit complex responses to changes in conditions. The biomolecule corona composition may be dependent on properties of the surface it is being formed on, wherein the properties of the surface may be dependent on changes in condition. For example, the zeta potential of a surface may be dependent on the compositions of the solvent (e.g., concentrations of water, additives, salts, and pH). Further, the biomolecule corona composition may be dependent on properties of biomolecules in the sample. Each biomolecule in a sample may comprise unique interaction potentials that can be modified by other biomolecules in the sample. The compositions of biomolecule coronas may fit poorly analytical models, making it difficult to determine true sample characteristics from measured biomolecule corona compositions.

Accordingly, determining true biomolecule concentrations may sometimes require targeted spike-in assays, in which analytes of interest are serially doped into samples to generate an empirical response curve. This method can be time and resource intensive (for example, a rare plasma protein may cost thousands of dollars per milligram) and may have limited sensitivities and measurement ranges.

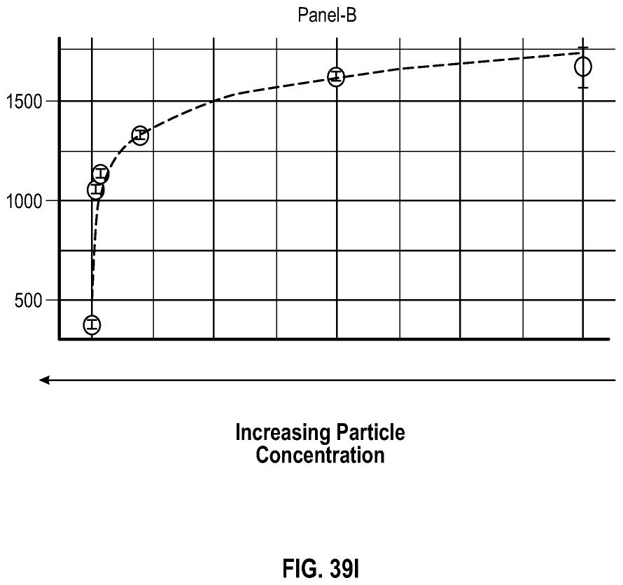

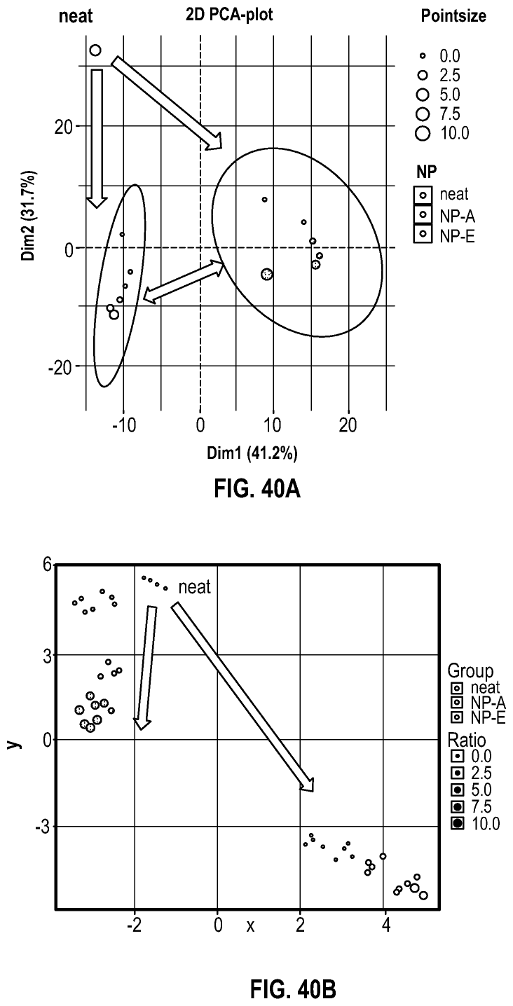

The present disclosure identifies a number of multi-condition assays which can generate smooth and systematic responses for biomolecules or groups of biomolecules from complex samples. For example, as shown in FIGS. 40A-C and 42, the dependence of biomolecule corona composition on the particle type and particle concentration shows a smooth curve. These trends may be used to optimize biomolecule collection. These trends may be used to quantify analyte concentration in the original sample based on the biomolecule corona composition. The PCA shown in FIG. 40A for NP-E and NP-A particles may be used as a reference curve to convert biomolecule corona composition to real sample concentration. Accordingly, the present disclosure provides a range of methods for quantifying true analyte concentration (e.g., of a particular protein or a collection of proteins) with biomolecule corona analysis.

In one aspect, the present disclosure provides a method for determining a concentration or an amount of a biomolecule or biomolecule group in a biological sample, the method comprising: contacting the biological sample with a plurality of particle-containing solutions each having a different particle concentration, to generate a plurality of biomolecule coronas that correspond to an individual solution of said plurality of particle-containing solutions; assaying the plurality of biomolecule coronas for a dataset comprising data corresponding to one or more biomolecules or biomolecule groups comprising the biomolecule or biomolecule group in the biological sample; and determining the concentration or the amount of the biomolecule or the biomolecule group in the biological sample based at least partially on the dataset. The plurality of particle-containing solutions may comprise at least 3, at least 4, at least 5, at least 8, at least 10, or at least 12 solutions. The plurality of particle-containing solutions may comprise at most 12, at most 10, at most 8, at most 6, at most 5, at most 4, or at most 3 solutions. The plurality of particle-containing solutions may comprise the same particle or plurality of particles. In many cases, the determining is made in the absence of a reference biomolecule external or added to said biological sample, for example a protein native to the biological sample increased in concentration 100-fold for use as an internal calibrant. The biomolecule or the biomolecule group may be present at a concentration of less than about 10 g/ml, less than about 1 μg/ml, less than about 100 ng/ml, less than about 10 ng/ml, less than about 1 ng/ml, or less than about 100 pg/ml. As such, the method may be used to quantify the majority of plasma proteins, including μg/ml cytokines and peptide hormones.

Such a method may be amenable to a range of particle concentrations. A particle concentration may be a concentration of a particle in a solution prior to contacting the biological sample, an amount of dry particle added to the biological sample, or a ratio of particle amount (e.g., mass) to biological sample volume combined to form a biomolecule corona. For example, a multi-particle concentration assay may combine a single particle stock solution with different volumes of the biological sample to generate a range of particle concentrations. In some cases, the subset of the plurality of particle-containing solutions differs in particle concentration by at least 0.5 orders of magnitude, by at least 1 order of magnitude, by at least 1.5 orders of magnitude, by at least 2 orders of magnitude, by at least 2.5 orders of magnitude, or by at least 3 orders of magnitude. In some cases, the subset of the plurality of particle-containing solutions differs in particle concentration by at most 3 orders of magnitude, by at most 2.5 orders of magnitude, by at most 2 orders of magnitude, by at most 1.5 orders of magnitude, by at most 1 order of magnitude, or by at most 0.5 orders of magnitude. A solution of the plurality of particle-containing solutions may have a particle concentration of at least 0.1 μg/ml, at least 0.5 μg/ml, at least 1 μg/ml, at least 5 μg/ml, at least 10 μg/ml, at least 50 μg/ml, at least 100 μg/ml, at least 500 μg/ml, at least 1 mg/ml, at least 5 mg/ml, at least 10 mg/ml, at least 25 mg/ml, or at least 50 mg/ml. A solution of the plurality of particle-containing solutions may have a particle concentration of at most 0.5 μg/ml, at most 1 μg/ml, at most 10 μg/ml, at most 100 μg/ml, at most 1 mg/ml, at most 10 mg/ml, or at most 25 mg/ml. A solution of the plurality of particle-containing solutions may have a particle concentration of approximately equal to a total protein concentration of the biological sample. Solutions of the plurality of particle-containing solutions may have particle concentrations of about 1 μg/ml to about 100 mg/ml. Solutions of the plurality of particle-containing solutions may have particle concentrations of about 0.1 μg/ml to about 10 mg/ml. Solutions of the plurality of particle-containing solutions may have particle concentrations of about 1 μg/ml to about 1 mg/ml. Solutions of the plurality of particle-containing solutions may have particle concentrations of about 10 μg/ml to about 10 mg/ml. Contacting the biological sample with a particle-containing solution may comprise combining equal volumes of the biological sample and the particle-containing solution. For example, contacting the biological sample with the plurality of particle-containing solutions may comprise combining at most about 250 μL of said biological sample with at most about 250 μL of a particle-containing solution, or may comprise combining at most about 100 μL of said biological sample with at most about 100 μL of a particle-containing solution. Contacting the biological sample with the plurality of particle-containing solutions may comprise adding at least about 1 nL of plasma, at least about 10 nL of plasma, at least about 100 nL of plasma, at least about 1 μL of plasma, at least about 10 μL of plasma, at least about 100 μL of plasma, at least about 1 ml of plasma, at least about 10 mL plasma, or at least about 100 mL plasma per cm² of particle surface area to each solution of the plurality of particle-containing solutions. Contacting the biological sample with the plurality of particle-containing solutions may comprise adding between 100 nL and 100 mL of plasma per cm² of particle surface area to each solution of the plurality of particle-containing solutions.

A method for analyte concentration determination may use a single particle type or a plurality of particle types. Each of said particle-containing solutions may comprise the same particle, such that the plurality of biomolecule coronas are associated with a single particle type in solutions of said plurality of particle-containing solutions, or may comprise the same plurality of particles. In many cases, each particle-containing solution is contacted to the biological sample for an identical length of time. In some cases, the biomolecule coronas are assayed prior to reaching equilibrium with the biological sample.

In some cases, a dataset generated in a single assay may enable abundance determination for a plurality of biomolecules. For example, one or more biomolecules or biomolecule groups may comprise a plurality of biomolecules or biomolecule groups, and the method may comprise identifying a concentration of each of the plurality of biomolecules or biomolecule groups in the biological sample. In some cases, the biomolecule or biomolecule group comprises a plurality of human plasma proteins or human plasma protein groups, and the plurality of human plasma proteins or human plasma protein groups comprises at least 2, at least 4, at least 6, at least 8, at least 10, at least 12, at least 15, at least 20, at least 25, at least 40, at least 60, at least 100, at least 150, at or at least human plasma proteins or human plasma protein groups. In such cases, the concentrations of the human plasma proteins or human plasma protein groups may be determined at least partially based on intensities of said plurality of signals.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### EXAMPLES

The following examples are illustrative and non-limiting to the scope of the devices, methods, systems, and kits described herein.

### Example 1 SAMPLE DILUTION-EFFECTS ON BIOMOLECULE CORONA COMPOSITION

This example describes sample dilution effects on biomolecule corona composition in a proteomic assay utilizing particles for biomolecule collection.

To explore the effects of sample dilution on particle corona formation, 5 types of particles were separately contacted with 5 dilutions of a plasma sample formed by pooling CPD or CP2D plasma from 15 healthy subjects. The samples were held at a constant temperature of 37° C., and were either left undiluted or were diluted by factors of 20, 10, 4 or 2.5 in Tris-EDTA (TE) buffer for the experiments. The particles used in the assay had different compositions, sizes (as measured by dynamic light scattering (DLS)), size homogeneities (as measured by polydispersity index (PDI)), and charges (as measured by zeta potential). The five particle-types are summarized in TABLE 2.

TABLE 2
Particles Tested in Dilution Assay

| | | DLS | DLS | |
|---|---|---|---|---|
| Particle | | Size | PDI | Zeta |
| Name | Description | (nm) | (nm) | potential |
| NP-A | Carboxyl | 410 | 0.03 | −31.4 |
| | Functionalized Polystyrene | | | |
| NP-B | Dextran coating | 225 | 0.11 | −5 |
| NP-C | Silanol | 233 | 0.05 | −36.9 |
| NP-D | N-(3- | 232 | 0.30 | 20.9 |
| | Trimethoxysilylpropyl)diethyl- | | | |
| | enetriamine | | | |
| NP-E | Poly(dimethylaminopropyl- | 283 | 0.09 | 25.8 |
| | methacrylamide) | | | |

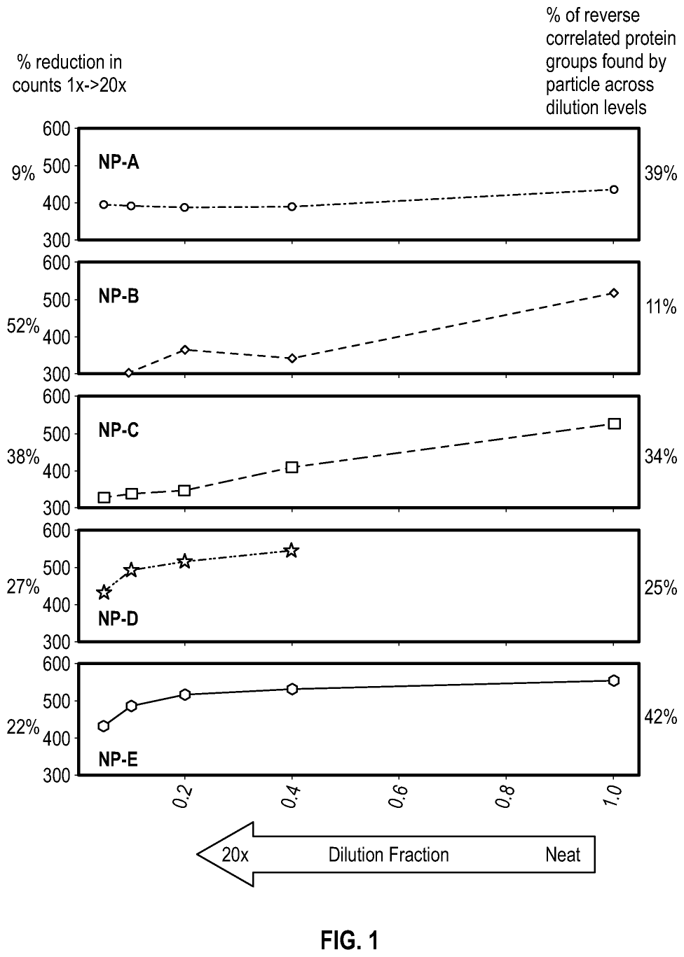

FIG. 1 shows the results of the dilution assays. Each plot provides aggregate protein adsorption data for a specific particle. The x-axes indicate the dilution of the sample, ranging from undiluted (1.0) to 20-fold diluted (0.05), while the y-axes indicates counts representative of biomolecules bound to each particle. As can be seen on the plots, the amount of protein bound to each particle diminished as the solution underwent dilution. However, the degree of decrease in protein adsorption varied between particle type. The dextran coated (NP-B) particles exhibited the greatest decrease in protein adsorption, exhibiting a 52% decrease in collected protein over a 10-fold dilution range, while the carboxyl functionalized polystyrene particles (NP-A) exhibited the smallest dilution effects, with total adsorbed protein diminishing by only 9% over a 20-fold dilution range.

The silanol particles (NP-C) exhibited a decrease in adsorbed protein content going from undiluted sample to 5-fold diluted sample, but a smaller decrease in protein adsorption levels in going from 5-fold to 20-fold diluted sample. The poly(dimethylaminopropylmethacrylamide) particles (NP-E) exhibited near invariance in protein adsorption levels in going from undiluted to a 5-fold diluted sample, but a more pronounced decline in protein adsorption in going from 5-fold to 20-fold dilution.

The assay also revealed that the relative concentrations of adsorbed proteins varied across dilutions. The percentage of proteins displaying opposite dilution trends was quantified for each type of particle (referred to herein as ‘reverse correlated protein groups’, and shown on the right side of FIG. 1). The poly(dimethylaminopropylmethacrylamide) particles (NP-E) had the highest percentage of reverse correlated protein groups (42%), while the dextran coated (NP-B) particles had the lowest percentage of reverse correlated protein groups (11%). For some particle types, the percentage of reverse correlated protein groups was greater than the change in total adsorbed protein across the measured dilution range.

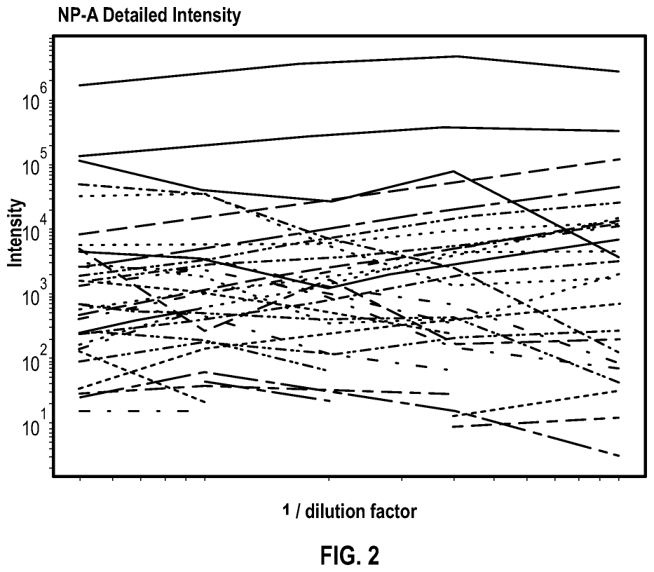

FIG. 2 shows dilution trends on the individual protein level for the carboxyl functionalized polystyrene particles (NP-A). Each trace in FIG. 2 corresponds to a different type of protein. While the total adsorbed protein content for these particles diminished by only 9% over the tested 20-fold dilution range, nearly 40% of the adsorbed proteins were reverse correlated. While some proteins exhibited near dilution-independent adsorption behavior (e.g., the topmost trace), other proteins exhibited complex dilution profiles.

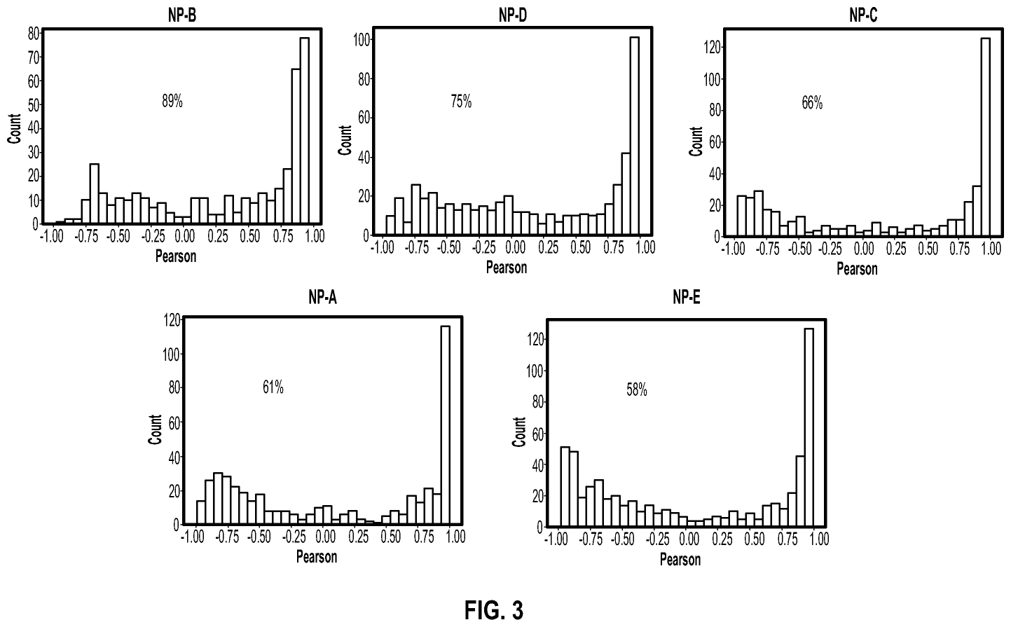

FIG. 3 provides Pearson correlation diagrams for the proteins identified in the coronas of the 5 particle types. The correlation score for a protein reflects the correspondence between dilution factor and particle corona abundance, with positive scores corresponding to increased abundance with decreased dilution. As can be seen from the five plots, most proteins have correlation scores close to 1, indicating that their particle corona abundance is strongly and inversely correlated with sample dilution. The number of negatively correlated proteins (proteins with negative correlation values) varies considerably between particle types. While only 11% of proteins have inversely correlated binding profiles for the dextran coated (NP-B) particles, more than ⅓ of proteins had inversely correlated binding profiles for the silanol (NP-C), carboxyl functionalized polystyrene (NP-A), and poly(dimethylaminopropylmethacrylamide) (NP-E) particles.

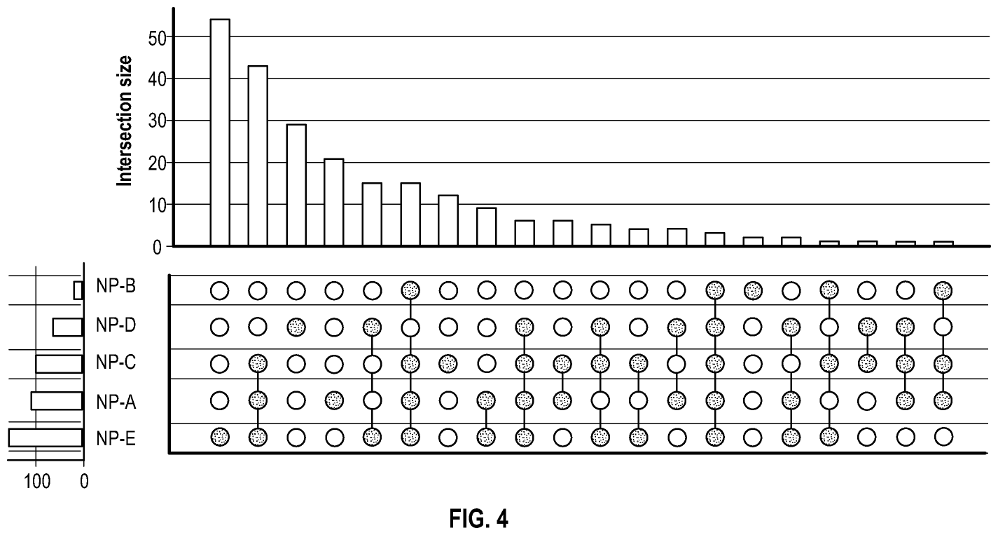

FIG. 4 shows the intersection sizes for the protein dilution profiles of the five particle types. As can be seen in the second column, the greatest degree of overlap is found between NP-E, NP-A, and NP-C, the particles with the greatest numbers of inversely correlated protein binding profiles. As is shown in columns 1 and 3, NP-E and NP-D have the least amount of behavioral overlap with other particle types, while NP-B (column 15) has the greatest overlap with other particle types.

### Example 2 EFFECT OF TOTAL PARTICLE SURFACE AREA ON BIOMOLECULE CORONAS

This example describes the effect of total particle surface area on biomolecule coronas in a proteomics assay involving protein collection on particles.

One important determinant for particle corona formation is the total surface area of particles in a sample. Particle coronas can contain multiple layers of proteins, each with different degrees of lability. For some particle types, proteins bind most strongly to the particle itself, and bind weakly to biomolecule layers surrounding the particle. Thus, the size of the combined surface area of all particles in a sample can have a large effect on the total amount of protein collected in particle coronas, as well as on the compositions of the coronas, themselves.

In this example, the total adsorbed protein content on 5 particle types (described in TABLE 2) was measured as a function of particle and protein concentration. The five particles varied based on a number of characteristics, including composition, size, and charge. Each experiment was performed by mixing a Tris-EDTA buffer containing a particular type of particle with human plasma. The concentrations of particles and protein as well as the aggregate particle surface area in each of these solutions is provided in TABLE 3, below. The samples were held at a constant temperature of 37° C. After a defined incubation period, the particles were collected, and the protein adsorbed to the particles was eluted and quantified.

TABLE 3
PARTICLE AND PROTEIN CONCENTRATIONS AND SURFACE AREAS

| | | | | | PARTICLE |
|---|---|---|---|---|---|
| | | | | | SURFACE |
| | | | | AGGRE- | AREA TO |
| | | | PROTEIN | GATE | SAMPLE |
| | TOTAL | PARTICLE | CONCEN- | PARTICLE | VOLUME |
| | VOLUME | MASS | TRATION | SURFACE | RATIO |
| | (μl) | (mg) | (M) | AREA (cm²) | (cm²/μl) |
| CONDITION | PARTICLE NP-C | | | | |
| 1 | 85 | 0.43 | 1.40 | 19.7 | 0.12 |
| 2 | 40 | 0.20 | 1.40 | 9.3 | 0.12 |
| 3 | 60 | 0.30 | 1.12 | 13.93 | 0.14 |
| 4 | 85 | 0.43 | 0.90 | 19.7 | 0.16 |
| 5 | 130 | 0.65 | 0.66 | 30.17 | 0.18 |
| CONDITION | PARTICLE NP-D | | | | |
| 1 | 85 | 0.43 | 1.40 | 20.6 | 0.12 |
| 2 | 40 | 0.20 | 1.40 | 9.7 | 0.12 |
| 3 | 60 | 0.30 | 1.12 | 14.51 | 0.15 |
| 4 | 85 | 0.43 | 0.90 | 20.6 | 0.16 |
| 5 | 130 | 0.65 | 0.66 | 31.43 | 0.63 |
| CONDITION | PARTICLE NP-E | | | | |
| 1 | 85 | 0.43 | 1.40 | 17.6 | 0.10 |
| 2 | 40 | 0.20 | 1.40 | 8.3 | 0.10 |
| 3 | 60 | 0.30 | 1.12 | 12.43 | 0.12 |
| 4 | 85 | 0.43 | 0.90 | 17.6 | 0.14 |
| 5 | 130 | 0.65 | 0.66 | 53.88 | 0.32 |
| CONDITION | PARTICLE NP-A | | | | |
| 1 | 85 | 0.21 | 1.40 | 17.11 | 0.10 |
| 2 | 40 | 0.10 | 1.40 | 8.05 | 0.10 |
| 3 | 60 | 0.15 | 1.12 | 12.08 | 0.12 |
| 4 | 85 | 0.21 | 0.90 | 17.11 | 0.14 |
| 5 | 130 | 0.33 | 0.66 | 26.17 | 0.52 |
| CONDITION | PARTICLE NP-B | | | | |
| 1 | 85 | 0.43 | 1.40 | 60.9 | 0.36 |
| 2 | 40 | 0.20 | 1.40 | 28.7 | 0.36 |
| 3 | 60 | 0.30 | 1.12 | 42.98 | 0.43 |
| 4 | 85 | 0.43 | 0.90 | 60.9 | 0.49 |
| 5 | 130 | 0.65 | 0.66 | 93.13 | 1.86 |

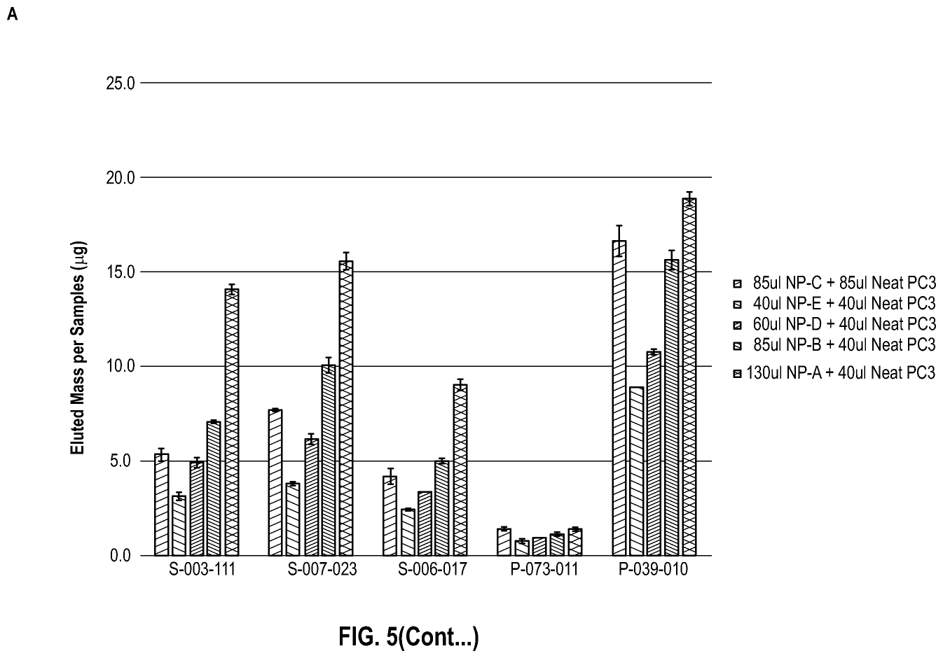

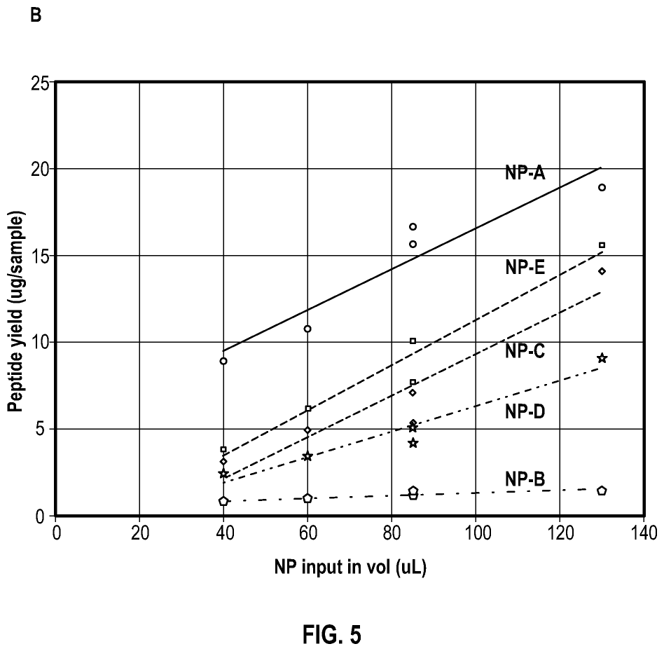

As can be seen in FIG. 5 panel A, the total protein adsorbed to the particles correlated with the number of particles added to each solution. However, the total amount of protein collected, as well as the dependence of the amount of protein collected upon particle concentration, varied between particle types. The total amount of protein collected varied from around 2 μg for the dextran coated particles (NP-B) to as much as 18 μg for the carboxyl functionalized polystyrene nanoparticles (NP-A).

As is shown in FIG. 5 panel B, the effect of increasing the number of particles (and thus the effect of increasing the aggregate particle surface area) on adsorbed protein mass varied between particles. For all five particle types, areas particle input was increased, peptide yield also increased. However, the extend of the increase in peptide yield varied considerably between particle types. For example, the total protein collected on the silanol particles (NP-C) increased from 3 μg to 14 μg upon increasing the particle sample input from 40 μl to 130 μl, contrasting with the 2-fold change in total protein collected for the carboxyl functionalized polystyrene nanoparticles over the same input volume increase.

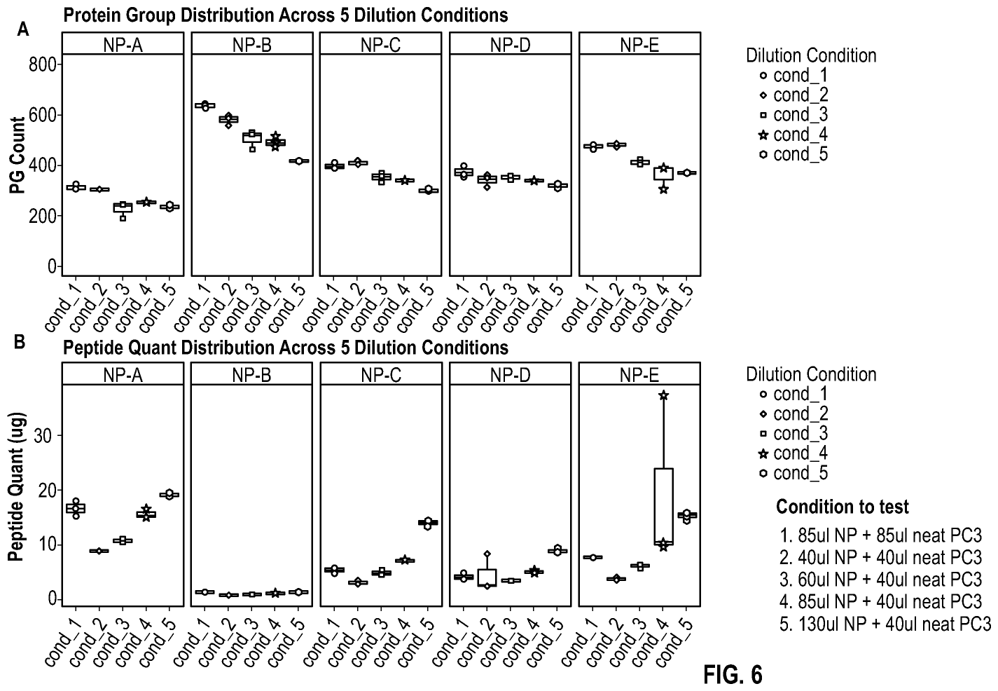

In addition to adsorbed protein quantity, the numbers of protein groups correlated with the particle-to-sample ratio. FIG. 6 panel A displays the results of mass spectrometric analysis on the particle-adsorbed protein, showing the number of distinct protein groups collected at each sample-to-particle ratio. As shown in panel A, the number of protein groups detected decreased as the ratio of particles-to-sample increased. As can be seen in FIG. 6 panel B, this is the inverse of the trend observed for total adsorbed peptide, which correlates positively with the particle-to-sample ratio. The carboxyl functionalized polystyrene particles (NP-B) simultaneously adsorbed the lowest mass and the greatest number of types of proteins amongst the panel, showing that total protein yield can be inversely proportional to adsorbed protein diversity.

At constant particle-to-sample ratios, the number of protein groups identified varied with total sample volume (condition 1 vs condition 2). The direction of this trend depended on particle type. For three types of particles (NP-A, NP-B and NP-D), the number of detected protein groups increased with sample volume. For two types of particles, the number of detected protein groups decreased with sample volume (NP-C and NP-E).

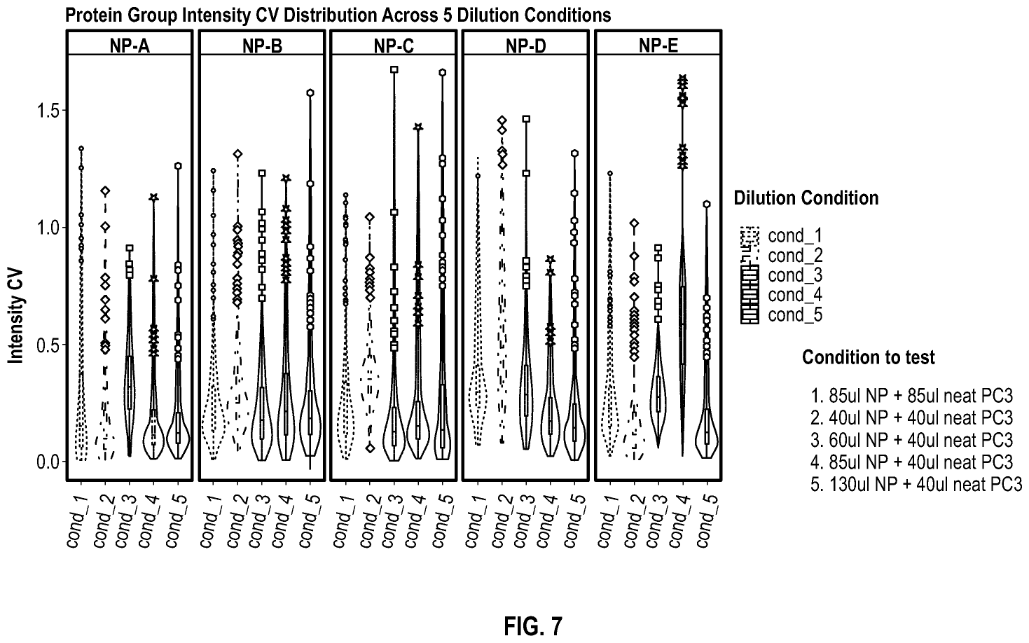

FIG. 7 shows the coefficients of variation (CV) for the protein groups detected on each particle type in the five dilution conditions. A low CV indicates consistent protein group abundance across replicates. FIG. 7 displays a wide range of protein group intensity CVs, indicating that some types of proteins adsorb with a high degree of stochastic variation.

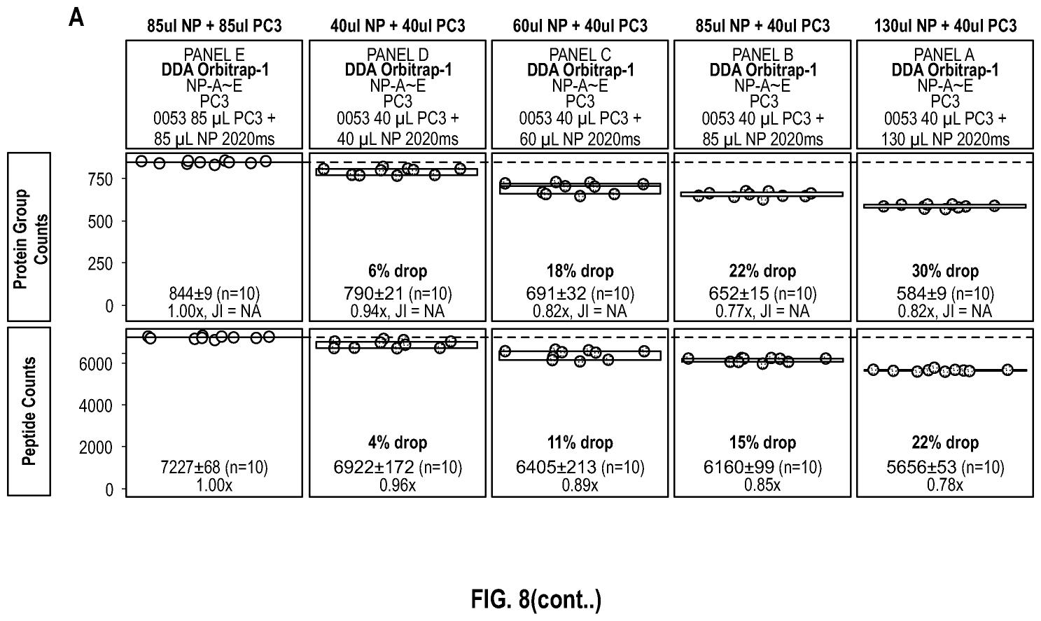

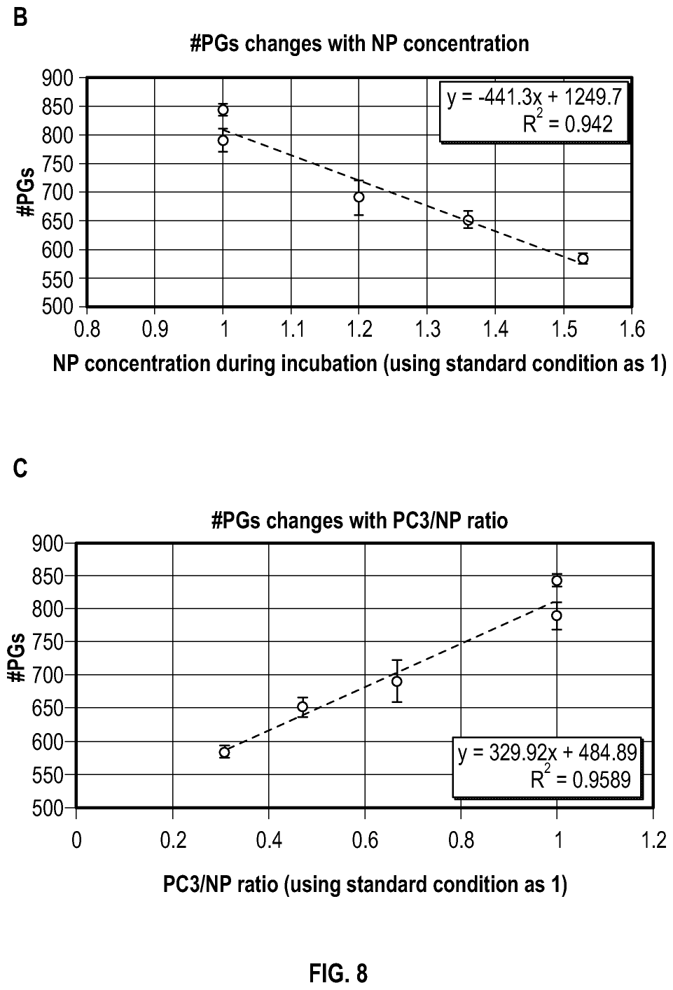

FIG. 8 shows an assay utilizing a panel having a combination of particle types. In this experiment, particle adsorbed protein was measured human plasma samples were combined in various volumes of a sample containing all five types of particles provided in TABLE 2 with 10 replicates performed at each volume ratio. Following incubation at 37° C., the particle-adsorbed protein was analyzed with mass spectrometry.

FIG. 8 panel A provides the total number of protein groups and the number of peptides adsorbed to the particle mixtures. As can be seen in panel B, the number of protein groups decreases as particle concentration increases. As can be seen in panel C, the number of protein groups increases as the sample to particle ratio increases.

Thus, the aggregate amount and number of types of proteins vary with aggregate particle surface area. For some particle types, these two variables correlate in the same direction. For other particle types, the diversity of proteins collected correlates negatively with the amount of protein adsorbed. Optimizing aggregate particle surface area can increase the profiling depth of a proteomic assay.

### Example 3 PARTICLE ELECTROSTATICS

This example describes particle electrostatics and covers the interdependence between particle charge and protein-affinity. One way in which sample conditions affect particle corona formation is by altering the free energies of binding between the protein and the particle. For example, a change in solution conditions that stabilizes the solubilized form of a protein may disfavor particle adsorption. In some cases, buffer conditions can attenuate effects imparted by electrostatic interactions, potentially changing the relative abundances of proteins within a particular corona.

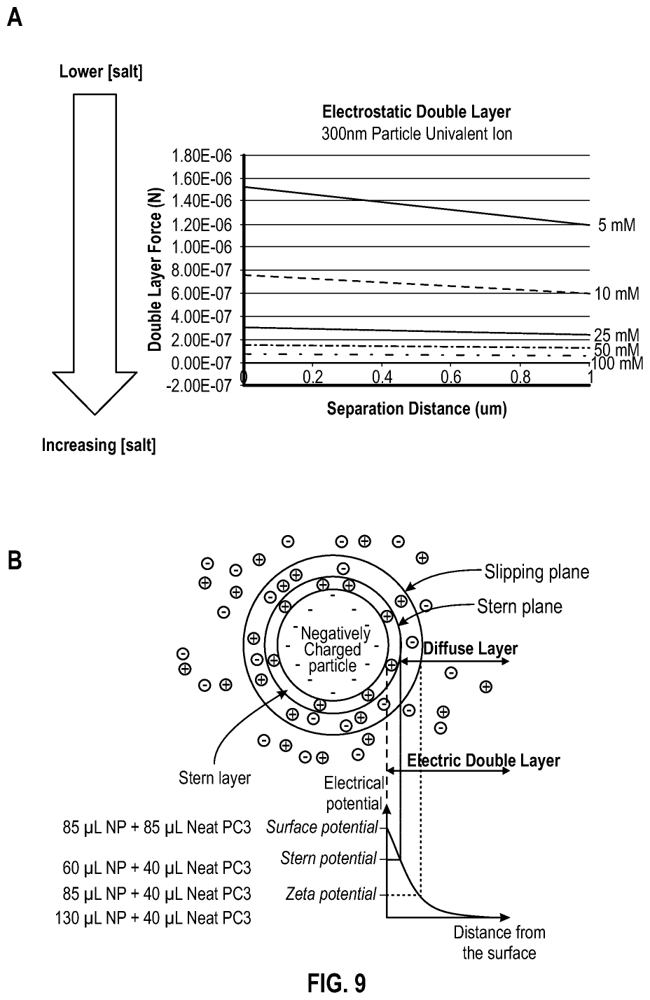

FIG. 9 summarizes a computational investigation of particle-solute interactions, in which 300 nm particles were modeled as univalent hard spheres surrounded by small ions. The double layer forces between the particle and ions were calculated over a range of separation distances spanning 0.2 to 1 μm, and over various ion concentrations ranging from 5-100 mM.

As can be seen from FIG. 9 panel A, the double layer force was larger and more responsive to separation distance for lower ion concentrations. These data suggest that diminishing charged solute concentration can improve a particle's solution stability, and act to strengthen the particle's charge-based interactions with proteins, thus leading to diminished protein binding specificity. Conversely, the results indicate that increasing charged solute concentration (e.g., increasing salt concentration) can destabilize a charged particle, leading to more specific particle-protein interactions. Panel B illustrates the multiple shells involved in charged solute-particle interactions.

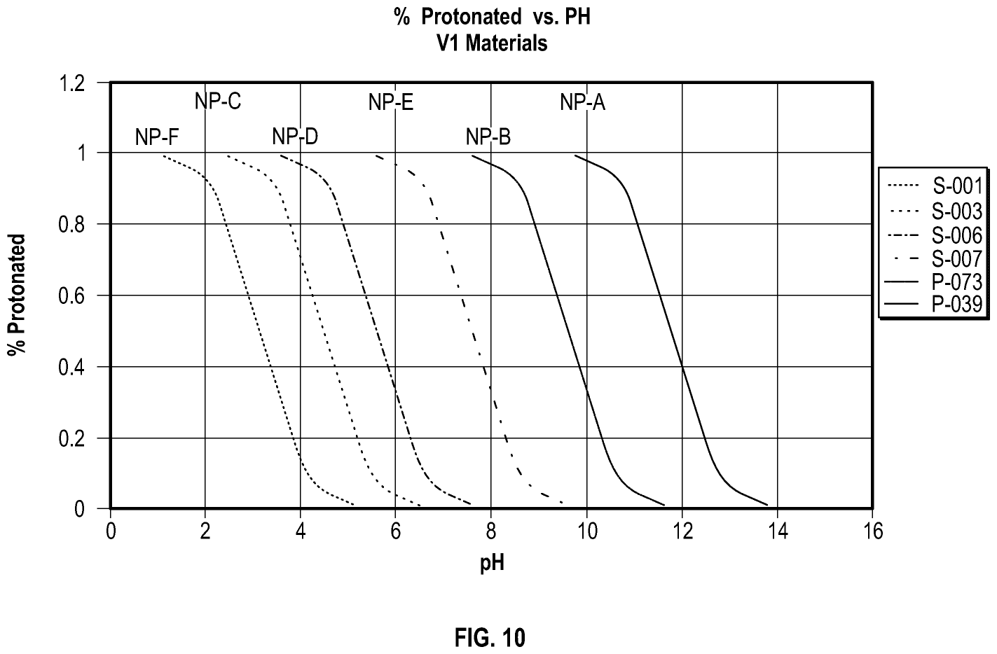

FIG. 10 shows protonation curves for multiple particle types (summarized in TABLE 4). As can be seen in the chart, the particle pKₐs vary based on particle composition and surface functionality, and range from around 3 to 11.5. Thus, at physiological pH, some particle types will be nearly completely deprotonated, while other will be zwitterionic or completely protonated. The 6 particles assayed have relatively narrow protonation profiles, indicating that particle surface charge can be modified by small changes in pH.

TABLE 4
Particle Types Measured in pH Titration

| | | DLS | DLS | |
|---|---|---|---|---|
| Particle | | Size | PDI | Zeta |
| Name | Description | (nm) | (nm) | potential |
| NP-F | Carboxylate | 374 | 0.23 | −34.0 |
| | (Citrate) | | | |
| NP-C | Silanol | 233 | 0.05 | −36.9 |
| NP-D | N-(3- | 232 | 0.30 | 20.9 |
| | Trimethoxysilyl- | | | |
| | propyl)diethylene- | | | |
| | triamine | | | |
| NP-E | Poly(di- | 283 | 0.09 | 25.8 |
| | methylamino- | | | |
| | propylmeth | | | |
| | acrylamide) | | | |
| NP-A | Carboxyl | 410 | 0.03 | −31.4 |
| | Functionalized | | | |
| | Polystyrene | | | |
| NP-B | Dextran coating | 225 | 0.11 | −5 |

### Example 4 Protein Compression Effects from Protein Corona Occupancy

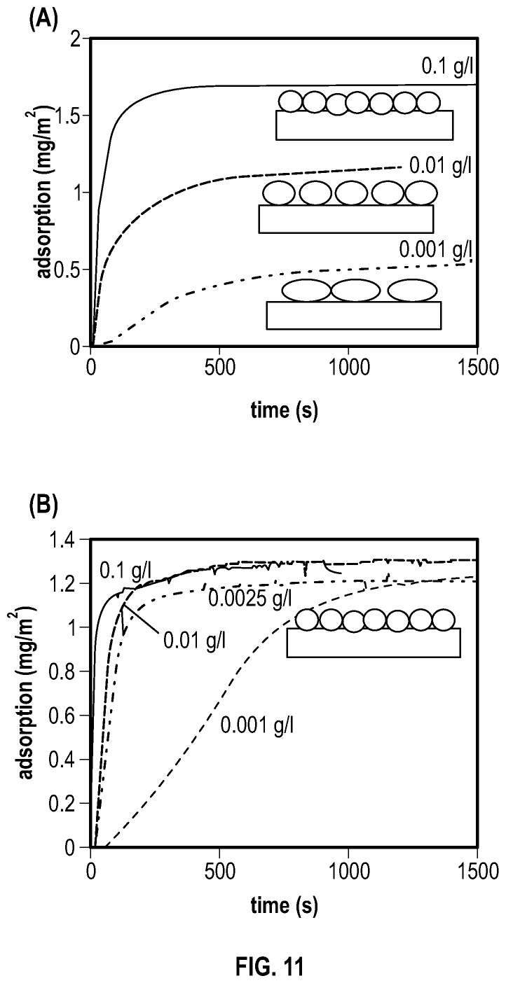

This example demonstrates the impact of aggregate particle surface area, sample volume, and analyte concentration on the amount of protein collected on particles. FIG. 11 graphically illustrates this principle. As is shown in panel A, when a particle's surface is sparsely populated, protein adsorption onto a particle surface proceeds according to Langmuir adsorption isotherm behavior. Thus, adsorption kinetics can be coupled to solute concentration and particle surface area. As shown in panel B, while final protein adsorption values are nearly invariant over a 100-fold concentration range, adsorption rate slows considerably as solute concentration is diminished.

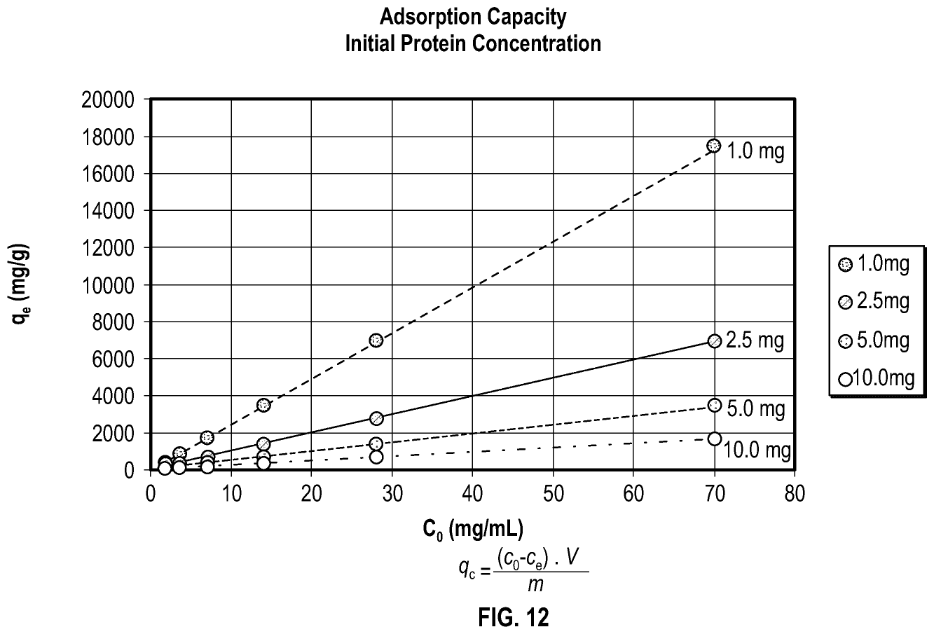

FIG. 12 graphically illustrates a series of protein-particle binding calculations, based on

$${q}_{e} = \frac{( {c}_{0} - {c}_{e} ) * V}{m} ,$$

the equilibrium binding equation where qₑ is equilibrium adsorption (mass protein adsorbed per mass of particle), C₀ is initial protein concentration, Cₑ is equilibrium protein concentration, V is sample volume, and m is particle mass. The calculations were performed with the assumption that protein binding was non-perturbing, and thus that Cₑ could be approximated as 0. C₀ was varied from 70 to 1.75 mg/mL, simulating protein concentrations for undiluted and 40-fold diluted plasma. The results show that high sample and particle concentrations can diminish the amount of protein adsorbed per particle. While equilibrium protein adsorption is predicted to increase with initial protein concentration, the calculations suggest that protein adsorption capacity will be inversely correlated with particle mass. Given that surface area to volume (and thus surface area to mass) are inversely correlated, this finding suggests that protein binding is driven by particle surface area, and that it may be possible to collect a greater number of proteins by using a lower concentration of particles.

### Example 5 Protein Corona Dependence on pH and Particle Surface

This example covers protein corona dependence on pH and particle composition.

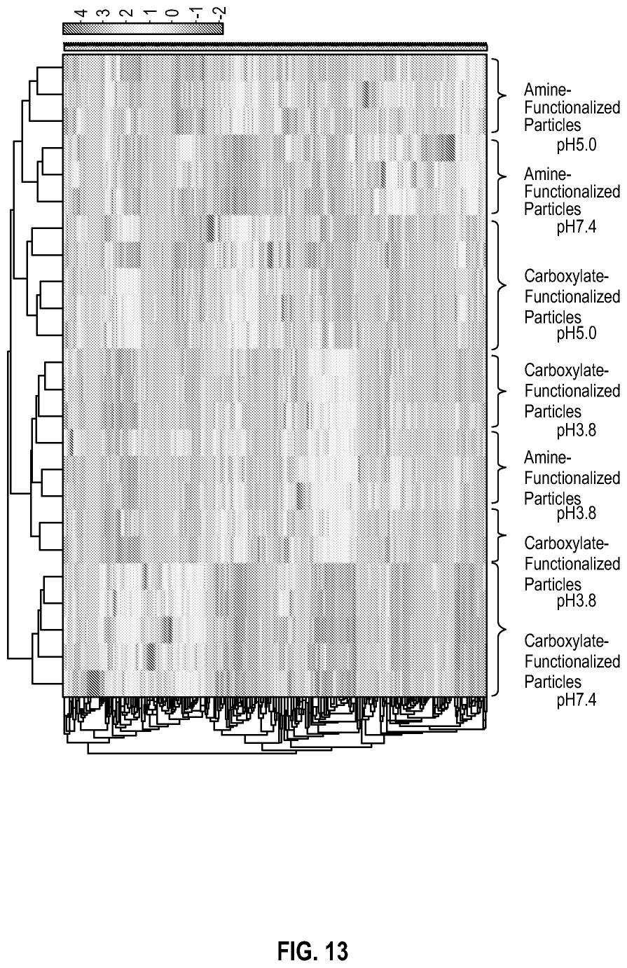

A series of protein corona formation assays were performed with a range of particles in varying pH samples. FIG. 13 provides a binding heat-map for a range of protein groups, with columns organized by pH and particle-type, and rows organized by protein group. Blue matrix entries indicate low protein group abundancies, while red entries indicate high particle corona occupancies. As can be seen on the chart, each particle type enriches a distinct set of protein groups. The chart also highlights how changes in pH can alter the protein corona compositions for a particular particle type. For example, the set of proteins most enriched by carboxylate functionalized particles at pH 7.4 (top left of chart) is almost entirely orthogonal to the set of proteins most strongly enriched by the amine functionalized particles at pH 5.0 (bottom right). This can in part be rationalized by differences in surface charges. At pH 7.4, carboxylic acid moieties will be deprotonated, causing carboxylate functionalized particles to have negatively charged surfaces. At pH 5.0, amines will be largely protonated, resulting in positive surfaces on amine-functionalized particles.

### Example 6 pH Dependent Particle Adsorption by Three Serum Proteins

This example covers pH dependence for protein binding to particles. In this example, the binding of three types of proteins to eight types of particles was measured as a function of pH. The particle types consisted of 5 carboxylate functionalized particles (NP-A, NP-I, NP-F, NP-C and NP-G) and 3 amine functionalized particles (NP-D, NP-E and NP-H). The three proteins has isoelectric points of 4.37 (cartilage oligomeric matrix protein (COMP)), 5.91 (pregnancy zone protein), and 9.53 (proteoglycan 4).

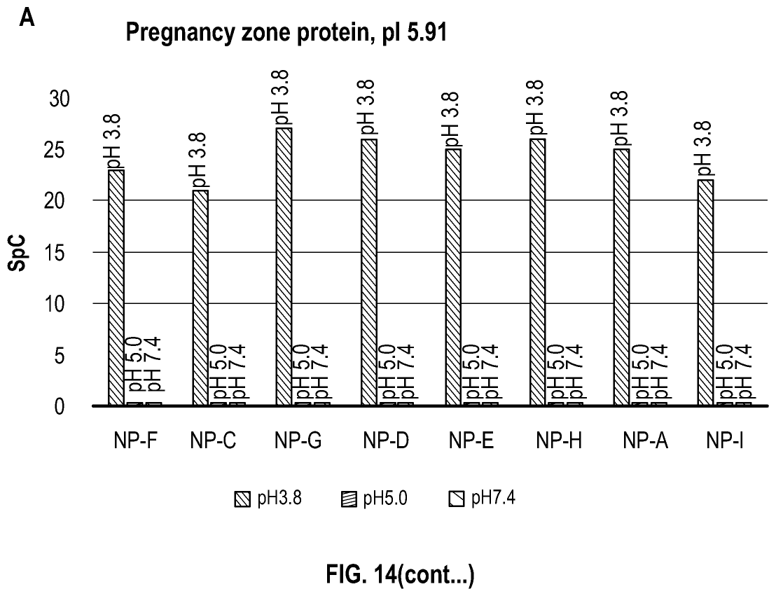

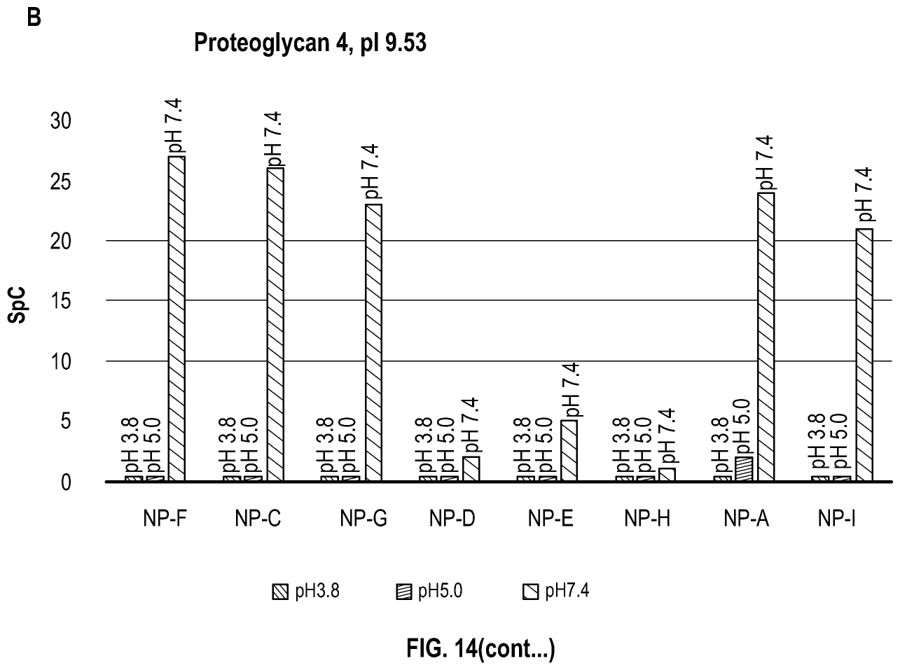

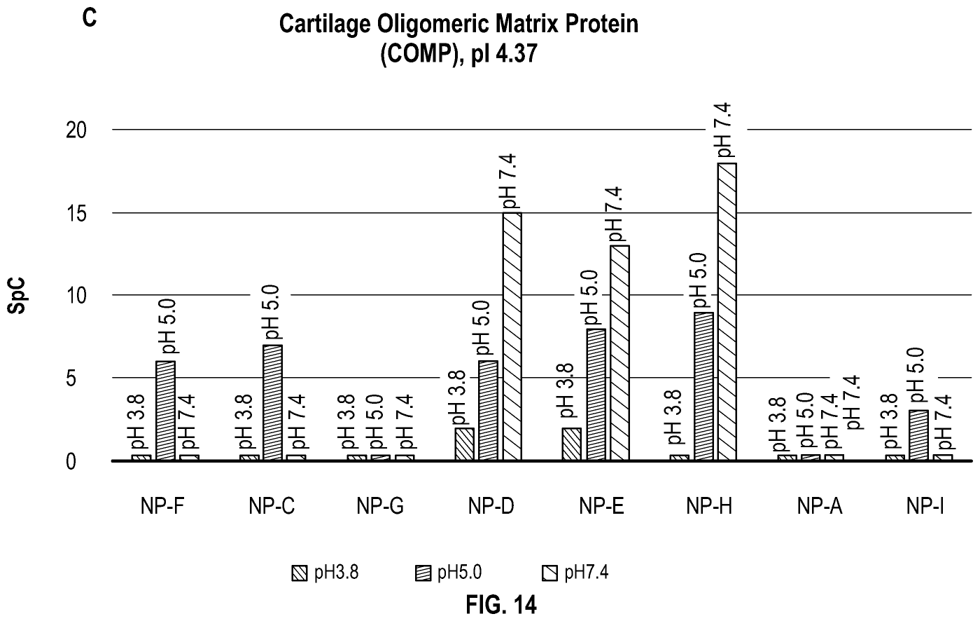

As can be seen in FIG. 14, all three proteins exhibited strongly pH-dependent binding profiles. Pregnancy zone protein (FIG. 14 panel A) only bound to the particles at a pH of 3.8, and did not bind to the particles at pH 5.0 or pH 7.4. This disparity highlights the complexity of protein-particle binding, as pregnancy zone protein required a pH well below its isoelectric point to bind to the particles. Both the amine functionalized particles and pregnancy zone protein were positively charged at this pH. Proteoglycan 4 (FIG. 14 panel B) displayed a similar type of pH dependence, only binding in high quantities at pH 7.4. However, unlike pregnancy zone protein, proteoglycan 4 bound in greater quantities to the carboxylate functionalized particles. COMP displayed the most complex binding behavior (FIG. 14 panel C), binding to the amine-functionalized particles in highest abundance at pH 7.4, and only binding to certain carboxylate-functionalized nanoparticles at pH 5.0.

### Example 7 Time Dependence to Protein Corona Formation

This example covers the time dependence to protein corona composition and formation. Protein coronas form through a dynamic exchange process, wherein proteins bind and desorb at rates partially defined by their binding affinities. As a corona composition changes over this dynamic exchange process, the binding affinities of particular proteins can undergo time dependent changes as well, further augmenting time-dependent changes in protein corona composition.

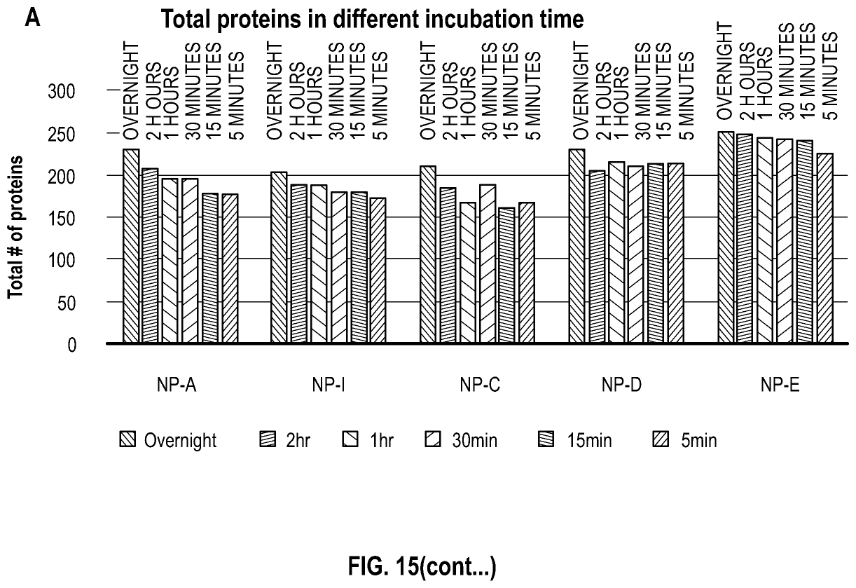

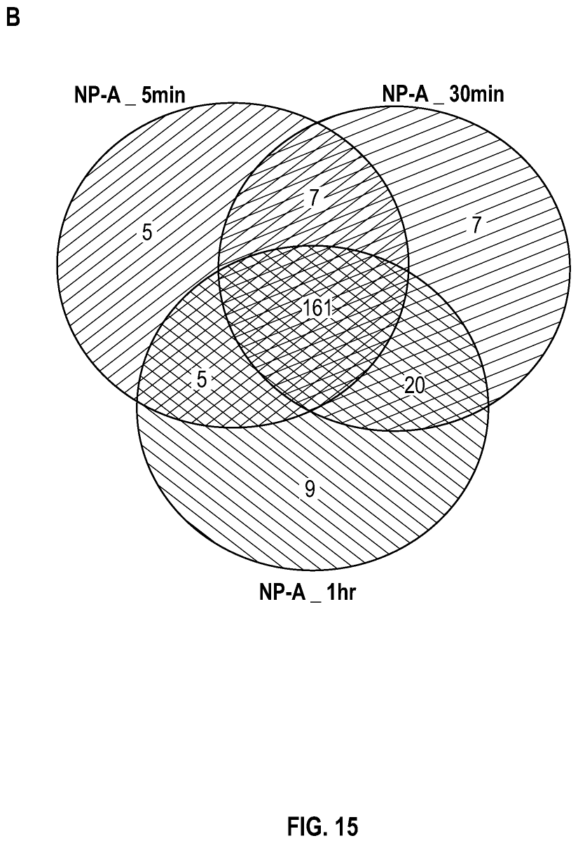

In this example, three carboxylate-functionalized particles (NP-A, NP-I and NP-C) and two amine-functionalized particles (NP-D and NP-E) were incubated with a complex biological sample, and assayed at different time points for protein corona content. As can be seen from FIG. 15 panel A, the total number of adsorbed protein-types increased over time for each of the five types of particles.

FIG. 15 panel B illustrates protein corona composition changes on the NP-A carboxylate-functionalized particles over the first hour of protein corona formation. Each area in the chart provides the number of types of proteins found within the protein coronas at each combination of time points. As can be seen from the diagram, a total of 21 types of proteins were uniquely found at either the 5 minute, 30 minute, or 1 hour time points, while 161 protein types were identified at all three time points. Thus, a particle can be assayed at different times to produce different biomolecule corona signatures.

### Example 8 Protein Corona Buffer-Dependence

This example details the influence of buffer system on particle corona composition. Solution stability can be heavily influenced by buffer type. Two buffer systems can impart drastically different protein solubilities, and thus act as a major determinant for whether a particular protein binds to a particle. In this example, human plasma protein binding to 5 particle types (detailed in TABLE 5) was measured in two separate buffer systems, Tris-EDTA/CHAPS/KCl and Citrate/CHAPS/KCl, and the resulting protein coronas were characterized by mass spectrometry.

TABLE 5
PARTICLES USED IN MULTI-BUFFER ASSAY

| | | DLS | DLS | |
|---|---|---|---|---|
| Particle | | Size | PDI | Zeta |
| Name | Description | (nm) | (nm) | Potential |
| NP-C | Silanol | 233 | 0.05 | −36.9 |
| NP-D | N-(3- | 232 | 0.30 | 20.9 |
| | Trimethoxysilylpropyl)diethylenetriamine | | | |
| NP-E | Poly(dimethyl aminopropyl | 283 | 0.09 | 25.8 |
| | methacrylamide) (Dimethylamine) | | | |
| NP-A | Carboxyl Functionalized Polystyrene | 410 | 0.03 | −31.4 |
| NP-B | Dextran coating | 169 | 0.07 | −5 |

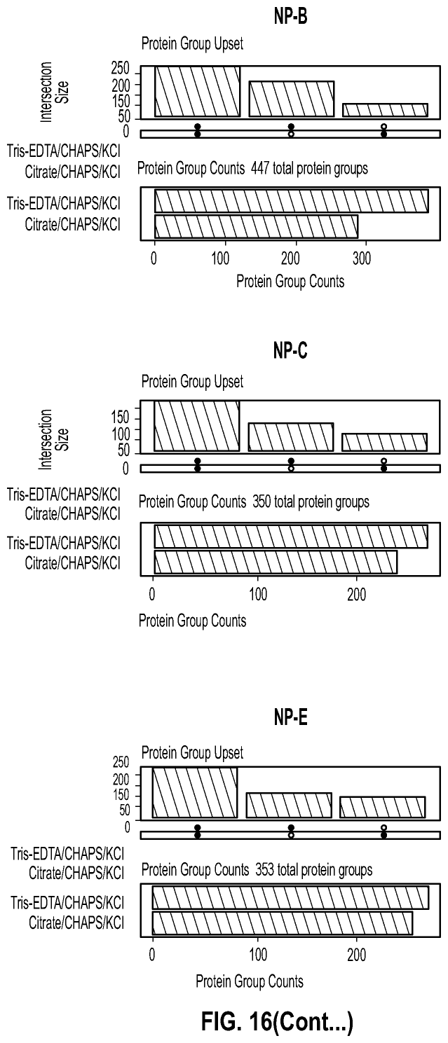

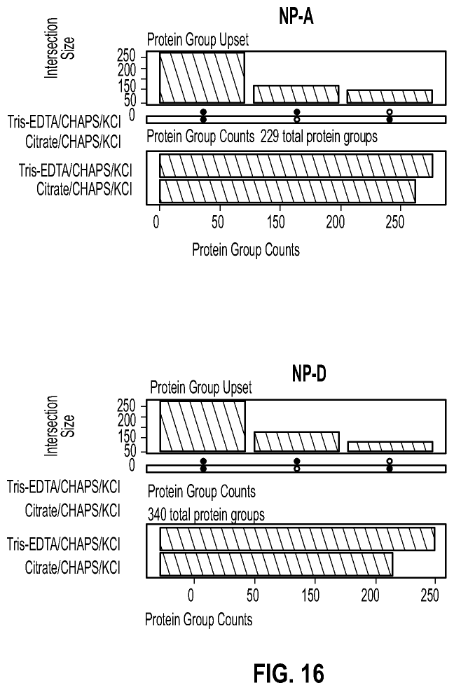

FIG. 16 outlines the results from this assay. In spite of the fact that the two buffer systems shared similar components, the overlap between protein groups collected on the particles in the two separate buffers shared as little as 51% and at most 63% similarity across the five particle types. The dextran coated particles (NP-B) exhibited the smallest overlap for collected protein groups between the two conditions at 51.2%. These particles also had the greatest disparity in the number of protein groups collected between the two conditions, with 387 protein groups collected in Tris-EDTA/CHAPS/KCl and 289 protein groups collected in Citrate/CHAPS/KCl. Other particles collected similar numbers of protein groups between the two conditions. For example, the carboxyl functionalized polystyrene particles collected 225 proteins in Tris-EDTA/CHAPS/KCl and 212 protein in Citrate/CHAPS/KCl. In spite of this similarity, only 167 of these protein groups were common between the two conditions.

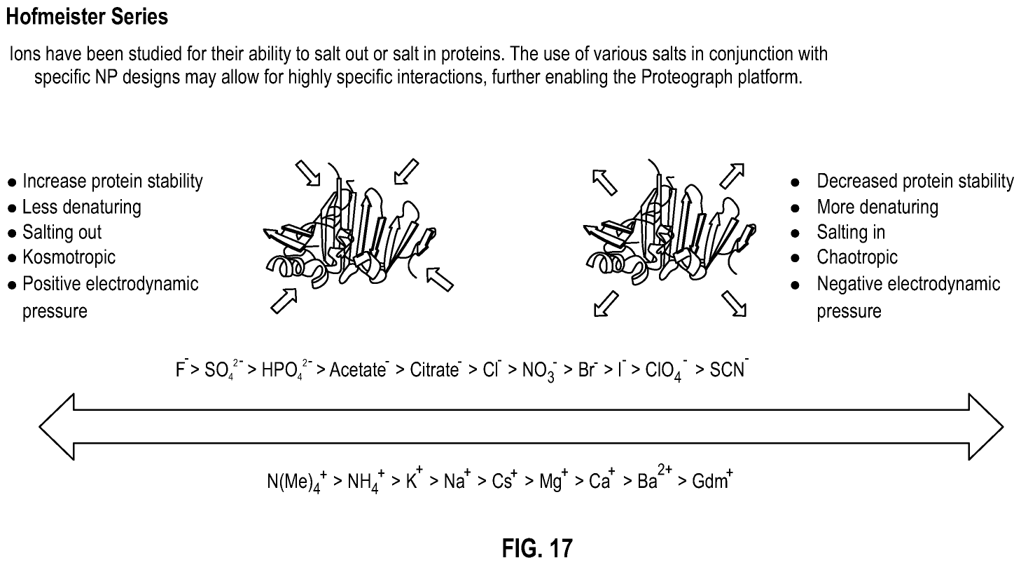

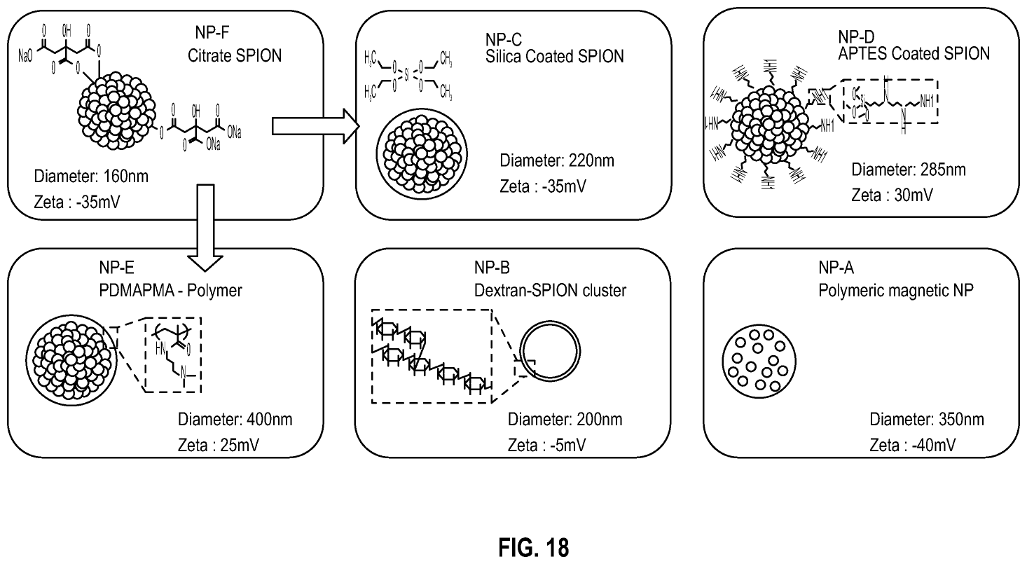

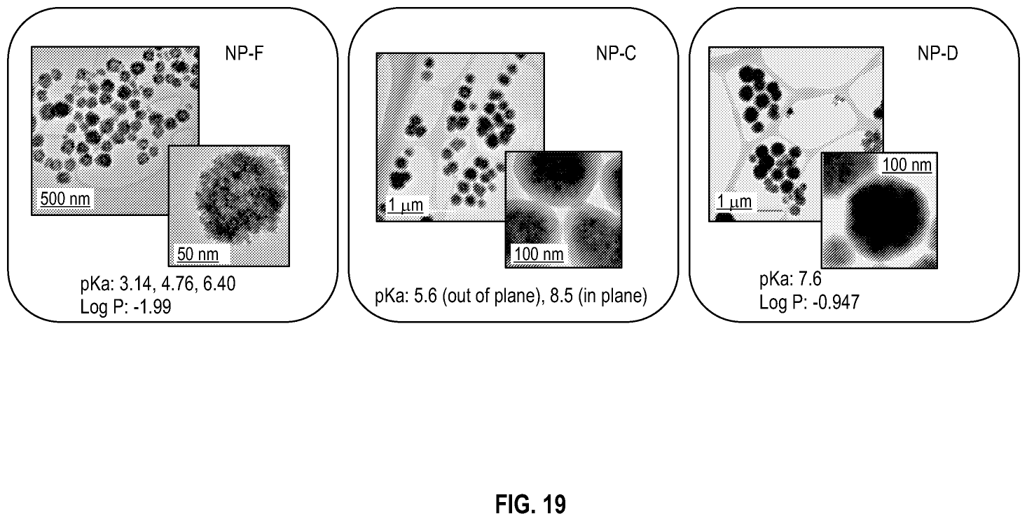

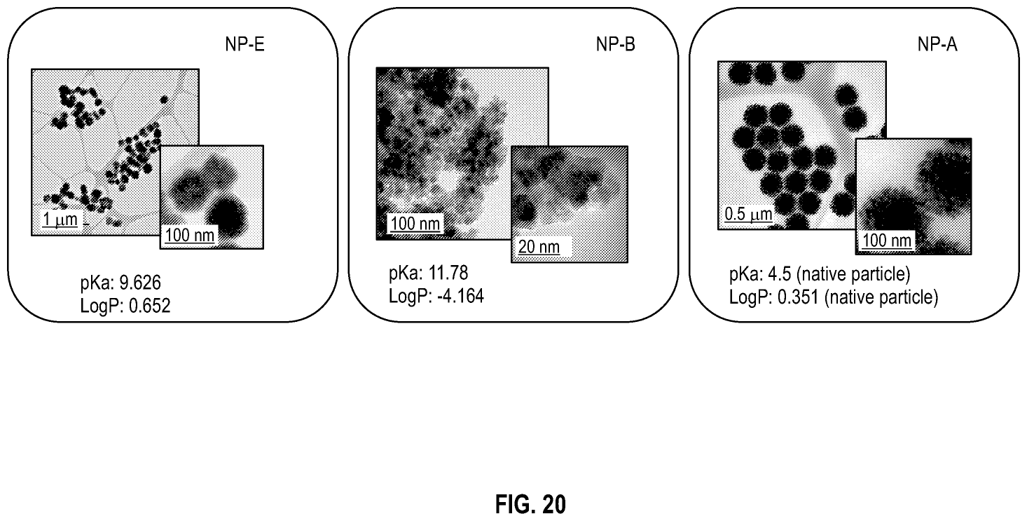

FIG. 17 illustrates a potential impact of salt-type on protein adsorption onto particles. A salt can stabilize or destabilize a protein in solution. Kosmotropic salts tend to provide positive electrodynamic pressure, which can increase protein solution-phase stability and diminish the degree of protein adsorption onto particles. Chaotropic salts tend to provide negative electrodynamic pressure, which can decrease protein solution-phase stability and promote protein adsorption onto particles. The magnitude of these effects not only depends on salt-type and concentration, but will also differ for each type of protein in a sample. Thus, the makeup of a protein corona can be manipulated by adjusting the concentrations and types of salts added to a solution with nanoparticles.

### Example 9 Tailoring Aggregate Substrate Surface Area to Maximize Adsorbed Protein Diversity

This example describes effects of aggregate substrate surface and sample volume on the diversity and quantity of adsorbed biomolecules. Varying the surface area of a substrate can change the quantity, relative concentrations, and number of types of biomolecules that adsorb to its surface. These responses are substrate- and biomolecule-type dependent. As disclosed herein, optimizing the surface area of substrates can allow for unbiased enrichment of biomolecules from a sample.

Biomolecule corona size and composition were measured for five types of particles (summarized in TABLE 2) combined with human plasma in five different volume combinations. The five combinations provided three different particle surface area to total volume ratios. The parameters for each experiment are summarized in TABLE 6 below. Following incubation at 37° C., the particles were collected, and the protein adsorbed to the particles was eluted and subject to mass spectrometric and BCA assay analysis to determine the number of protein groups and the total mass of the adsorbed proteins, respectively.

TABLE 6
SAMPLE VOLUME AND PARTICLE SURFACE AREA

| | | | | | PARTICLE |
|---|---|---|---|---|---|
| | | | | | SURFACE |
| | | | | AGGRE- | AREA TO |
| | | | PROTEIN | GATE | SAMPLE |
| | TOTAL | PARTICLE | CONCEN- | PARTICLE | VOLUME |
| | VOLUME | MASS | TRATION | SURFACE | RATIO |
| | (μl) | (mg) | (M) | AREA (cm²) | (cm²/μl) |
| CONDITION | PARTICLE NP-C | | | | |
| 1 | 170 | 0.43 | 1.4 × 10⁻³ | 19.7 | 0.12 |
| 2 | 80 | 0.20 | 1.4 × 10⁻³ | 9.3 | 0.12 |
| 3 | 125 | 0.43 | 9.0 × 10⁻⁴ | 19.7 | 0.16 |
| 4 | 50 | 0.13 | 1.4 × 10⁻³ | 5.80 | 0.12 |
| 5 | 80 | 0.40 | 1.4 × 10⁻³ | 18.6 | 0.23 |
| CONDITION | PARTICLE NP-D | | | | |
| 1 | 170 | 0.43 | 1.4 × 10⁻³ | 20.6 | 0.12 |
| 2 | 80 | 0.20 | 1.4 × 10⁻³ | 9.7 | 0.12 |
| 3 | 125 | 0.43 | 9.0 × 10⁻⁴ | 20.6 | 0.16 |
| 4 | 50 | 0.13 | 1.4 × 10⁻³ | 6.04 | 0.12 |
| 5 | 80 | 0.40 | 1.4 × 10⁻³ | 19.3 | 0.24 |
| CONDITION | PARTICLE NP-E | | | | |
| 1 | 170 | 0.43 | 1.4 × 10⁻³ | 17.6 | 0.10 |
| 2 | 80 | 0.20 | 1.4 × 10⁻³ | 8.3 | 0.10 |
| 3 | 125 | 0.43 | 9.0 × 10⁻⁴ | 17.6 | 0.14 |
| 4 | 50 | 0.13 | 1.4 × 10⁻³ | 5.18 | 0.10 |
| 5 | 80 | 0.40 | 1.4 × 10⁻³ | 16.6 | 0.21 |
| CONDITION | PARTICLE NP-A | | | | |
| 1 | 170 | 0.43 | 1.4 × 10⁻³ | 17.1 | 0.10 |
| 2 | 80 | 0.20 | 1.4 × 10⁻³ | 8.1 | 0.10 |
| 3 | 125 | 0.43 | 9.0 × 10⁻⁴ | 17.1 | 0.14 |
| 4 | 50 | 0.13 | 1.4 × 10⁻³ | 5.03 | 0.10 |
| 5 | 80 | 0.40 | 1.4 × 10⁻³ | 16.1 | 0.20 |
| CONDITION | PARTICLE NP-B | | | | |
| 1 | 170 | 0.43 | 1.4 × 10⁻³ | 60.9 | 0.36 |
| 2 | 80 | 0.20 | 1.4 × 10⁻³ | 28.7 | 0.36 |
| 3 | 125 | 0.43 | 9.0 × 10⁻⁴ | 60.9 | 0.49 |
| 4 | 50 | 0.13 | 1.4 × 10⁻³ | 17.91 | 0.36 |
| 5 | 80 | 0.40 | 1.4 × 10⁻³ | 57.3 | 0.72 |
| CONDITION | COMBINED PARTICLE PANEL | | | | |
| 1 | 170 | 0.43 | 1.4 × 10⁻³ | 27.2 | 0.16 |
| 2 | 80 | 0.20 | 1.4 × 10⁻³ | 12.8 | 0.16 |
| 3 | 125 | 0.43 | 9.0 × 10⁻⁴ | 27.2 | 0.22 |
| 4 | 50 | 0.13 | 1.4 × 10⁻³ | 8.0 | 0.16 |
| 5 | 80 | 0.40 | 1.4 × 10⁻³ | 25.6 | 0.32 |

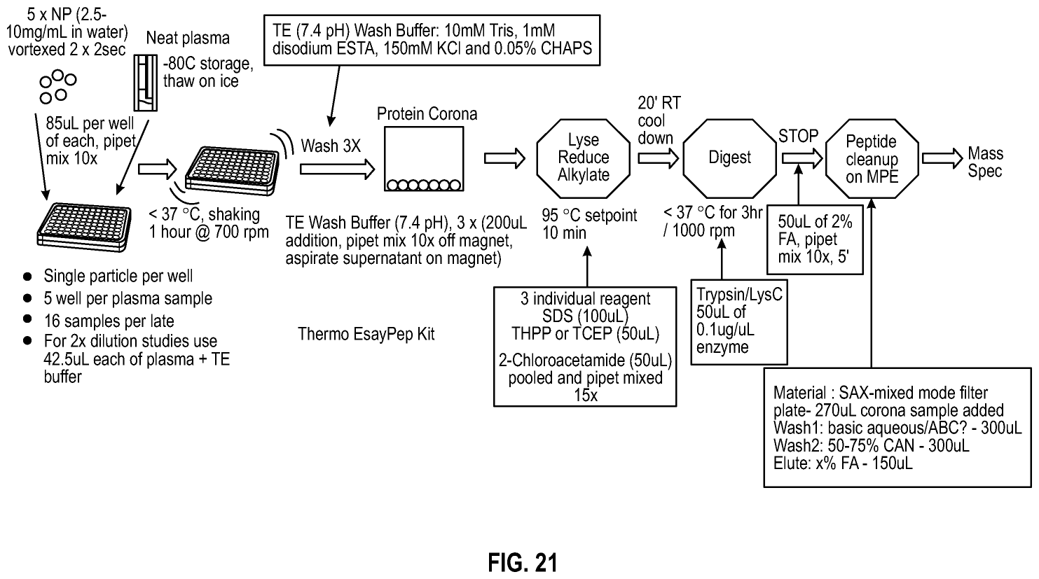

The results of the experiment are shown in TABLE 7 and FIG. 21. As can be seen from the data, the amount of protein adsorbed onto particles correlates with the aggregate particle surface area for all five particle types. The relationship between these two parameters can depend on particle type. Conditions 1, 3, and 5 provide similar amounts particle surface area, but can lead to protein mass collections differing by more than a factor of 2. For example, in going from condition 3 to condition 5, the quantity of protein collected on the carboxy functionalized styrene (NP-A) particles diminished from 4.4 to 2.1 μg/sample, while for the silanol particles (NP-C), the quantity of adsorbed protein increased from 6.8 to 8.6 μg/sample. The difference between conditions 3 and 5 is total sample volume. This shows that two particles can have opposite responses to changes in sample volume. Across the five conditions, protein group counts (i.e., the total number of types of proteins adsorbed to a sample of particles) correlated with higher sample volume and lower aggregate particle surface areas.

The number of different protein groups collected and identified by a combined particle panel (comprising equal amounts of each of the five types of particles) was measured with five different sample conditions. As can be seen from TABLES 6 & 7, the conditions that facilitated the greatest number of protein groups being collected and identified utilized the lowest particle surface area to sample volume ratios. For many of the individual particles tested, these conditions resulted in low protein mass recoveries, highlighting that the total amount of protein collected and the diversity of proteins collected from a sample can be inversely correlated, and that this relationship can be optimized to increase the depth at which a sample is profiled.

TABLE 7
PROTEIN GROUP COUNTS AND ADSORBED PEPTIDE
MASSES FOR EACH CONDITION ASSAYED

| | | | | | | COMBINED |
|---|---|---|---|---|---|---|
| | | | | | | PARTICLE |
| | PARTICLE | PARTICLE | PARTICLE | PARTICLE | PARTICLE | PANEL |
| | NP-C | NP-D | NP-E | NP-A | NP-B | Protein |
| | Peptide Mass (μg/Sample) | | | | | Group Count |
| Condition 1 | 7.6 | 20.3 | 5.6 | 2.1 | 16.9 | 577 |
| Condition 2 | 3.8 | 9.0 | 2.7 | 1.5 | 8.5 | 572 |
| Condition 3 | 6.8 | 19.5 | 7.2 | 4.4 | 17.3 | 568 |
| Condition 4 | 4.1 | 6.6 | 2.4 | 2.1 | 7.6 | 467 |
| Condition 5 | 8.6 | 16.2 | 6.1 | 2.1 | 16.6 | 465 |

### Example 10 Effect of Diminishing Particle Concentration on Biomolecule Corona Composition

This example details the relationship between particle concentration and biomolecule corona composition for two types of particles. In some cases, diminished particle concentration can increase the diversity of biomolecules collected on particles, and can enhance sample fractionation across multiple particle types.

Poly(dimethylaminopropylmethacrylamide) particles (NP-E, TABLE 2 row 6) and carboxyl functionalized polystyrene particles (NP-A, TABLE 2 row 2) were contacted to human plasma at particle: plasma ratios spanning wide concentration ranges. Each experiment was performed by mixing a Tris-EDTA buffer containing NP-E or NP-A particles with human plasma. The samples were prepared with 6 separate particle concentrations spanning a wide concentration range. The samples were incubated at 37° C. for one hour, allowing for biomolecule corona formation. Unbound biomolecules were then separated from the particles through a series of wash steps utilizing magnetic particle immobilization. Proteins were digested from the biomolecule coronas with an initial 10-minute 95° C. wash step followed by a 3-hour 37° C. trypsin digestion. The supernatant containing the resulting peptides was purified through solid-phase extraction, and then analyzed by LC-MS in a TimsTOF Pro using 30 min gradients on DDA mode and DIA mode. Parallel mass spectrometric analyses were performed on neat plasma at varying degrees of dilution.

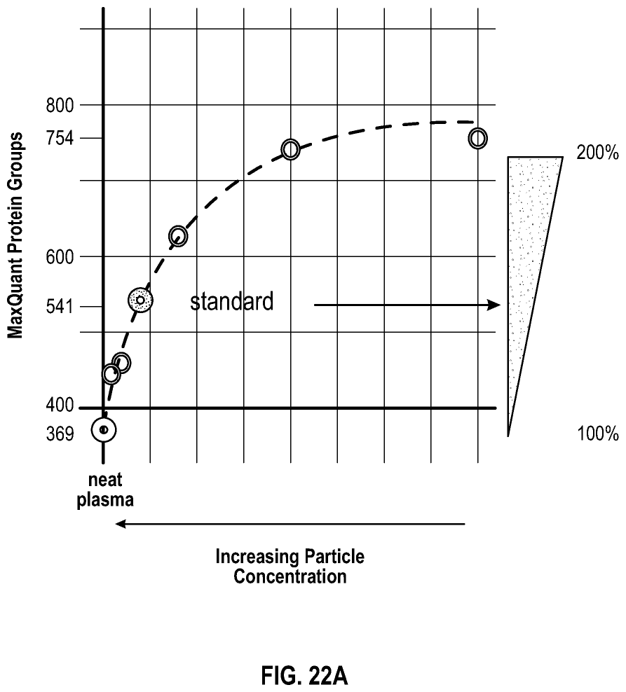

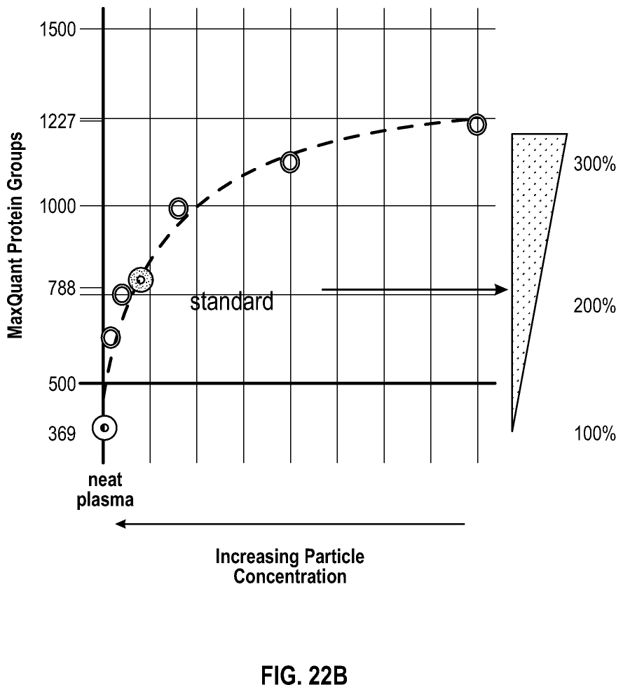

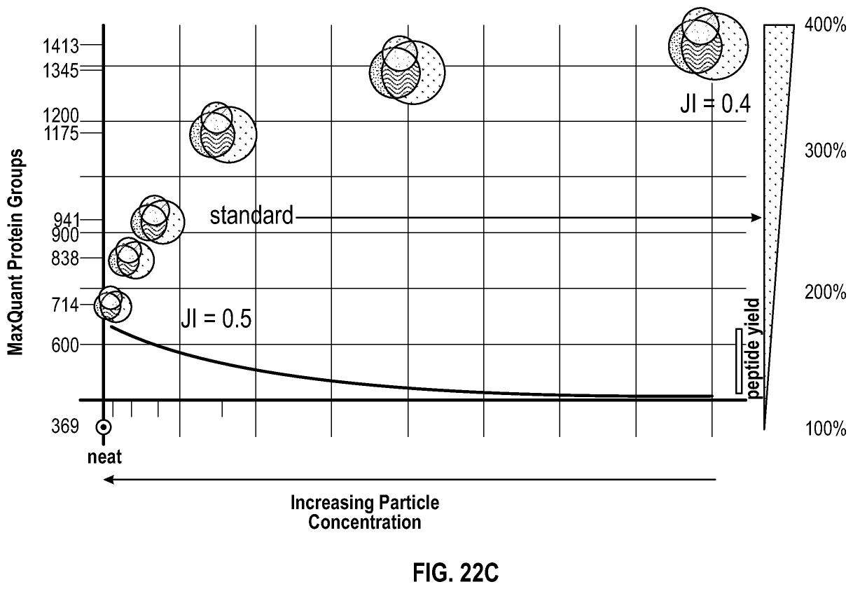

FIGS. 22A-D summarize results of the multifold dilution assays. FIGS. 22A-B provide the number of identified protein groups (y-axes) as a function of plasma: particle ratio (x-axis), with neat plasma (containing no particles) indicated as a plasma: particle ratio of zero. FIG. 22A provides results for carboxyl functionalized polystyrene particles (NP-A), while FIG. 22B provides results for Poly(dimethylaminopropylmethacrylamide) particles (NP-E). Diminished particle concentration correlated with increased protein identification for both particle types, with nearly twice as many proteins identified with the lowest particle concentration than with the highest particle concentration. Nonetheless, even the highest particle concentrations generated higher protein group counts than direct analysis of the neat plasma samples.

FIG. 22C depicts the overlap between the types of proteins identified on each particle at each dilution factor with the types of proteins identified from neat plasma. At each dilution level, the size of the bottom left circle indicates the number of proteins identified on NP-A particles, the size of the bottom right circle indicates the number of proteins identified on NP-E particles, and the size of the top circle indicates the number of proteins identified in neat plasma. Overlap between circles depicts the number of commonly identified proteins. As can be seen from the figure, the number of protein groups identified on each of the two particle types increases as particle concentration is diminished. Furthermore, variation between the types of proteins identified on each particle increased as particle concentration diminished. The trace at the bottom of the plot provides peptide yield at each dilution factor from the particle assays. While the trace shows that peptide yield (the overall mass of peptides collected on particles contacted to a sample) increases with increasing particle concentration, the per-particle diversity (number of distinct peptides) increases with decreasing concentration.

### Example 11 Multi-Concentration Particle Assay

This example overviews a method for fractionating and analyzing a biological sample by contacting the sample with multiple concentrations of particles. A biological sample comprising buffer-diluted human plasma is separated into multiple 200 uL portions. The portions of the biological sample are mixed with varying concentrations of POEGMA-coated paramagnetic particles. A first batch of samples are covered and incubated for 1 hour at 37° C. to allow for biomolecule corona formation on the particles. A second parallel batch of samples are covered and incubated for 2.5 hours at 37° C. to allow for biomolecule corona formation on the particles. Following the incubation times, the particles are separated from the portions of the biological sample, and the contents of their biomolecule coronas are analyzed.

The relationship between biomolecule corona complexity and incubation time is consistent with the Vroman effect, such that the samples with longer incubation times exhibit biomolecule coronas with greater biomolecule diversity. The overlap between the biomolecule corona compositions varies from 85-95% across different particle concentrations and from 78-90% across the two incubation times for samples with identical particle concentrations. Accordingly, the combination of biomolecule coronas provides a greater sample profiling depth than any of the biomolecule coronas taken individually. The combined dynamic range of the biomolecule coronas is 0.75 greater than the largest dynamic of the individual biomolecule coronas.

### Example 12 Particle Panel Dilution Assay

This example covers biological sample interrogation with a range of particle concentrations. Five types of particles (provided in TABLE 5) were combined with human plasma over a range of volume ratios corresponding to a large particle concentration range. Four replicates were performed for each combination of particle-type and concentration. The particles were provided in dry form, and reconstituted with deionized water to final total particle concentrations of 2.5-15 mg/ml. Human plasma underwent a 5-fold dilution with buffer, and then was mixed with the particle solutions at varying volume ratios to yield samples with constant particle mass and varying plasma volumes. The plates were sealed and incubated at 37° C. for 1 hour with shaking at 300 rpm. After incubation, the plate was placed on top of a magnetic collection device for 5 mins to draw down the particles. The supernatant, containing the non-corona unbound proteins was removed through a series of wash steps with 150 mM KCl and 0.05% CHAPS in a Tris EDTA buffer with pH of 7.4. Next, Lyse buffer was added to each sample and heated at 95° C. for 10 min with agitation at 1000 rpm. Trypsin was added to the samples for protein digestion. After 3 hours at 37° C. and 500 rpm shaking, the trypsin digestion was stopped by lowering sample pH. The nanoparticles were magnetically separated from the digested samples, and remaining supernatant was cleaned up with a filter cartridge (styrenedivinylbenzene reversed-phase sulfonate/SDB-RPS) kit. Peptide was twice eluted from the filter cartridge and combined. The peptides were analyzed with 30 and 120 minute LC-MS/MS runs on data-dependent acquisition mode and with 30 minute LC-MS/MS runs on data-independent acquisition mode.

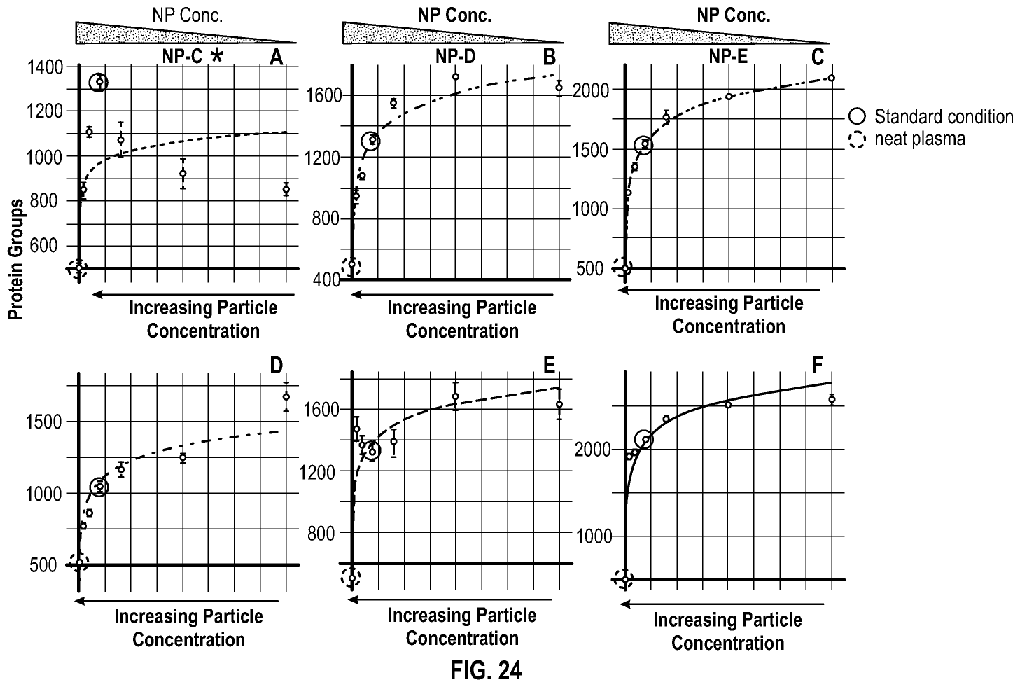

FIG. 24 summarizes protein group identifications obtained with a range of plasma-to-particle ratios for NP-C(panel A), NP-D (panel B), NP-E (panel C), NP-A (panel D), NP-B (panel E) and the 5-particle panel (panel F). The number of protein groups identified from neat plasma are provided as the furthest left data point on each plot. For NP-D, NP-E, NP-A, NP-Band the 5-particle panel, the number of identified protein groups increased with decreasing particle concentration. For NP-C, the highest protein group counts were obtained for an intermediary particle concentration. At all concentrations tested, all 5 particle types generated higher protein group counts than direct analysis of neat plasma.

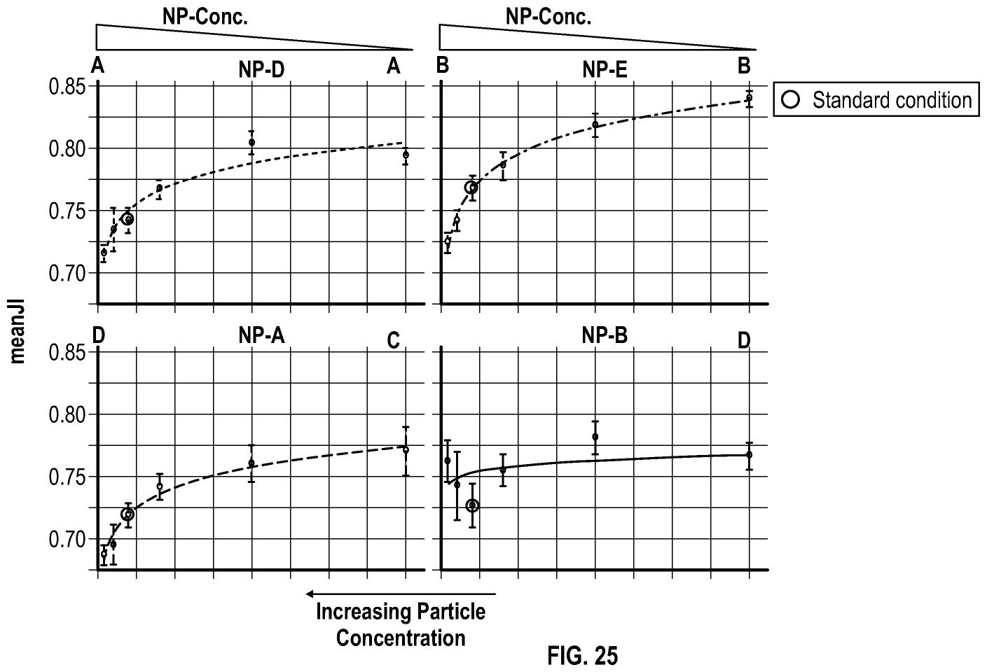

FIG. 25 provides Jaccard Similarity Coefficients (JI) for the 4 assay replicates at each concentration for NP-D (Panel A), NP-E (Panel B), NP-A (Panel C), and NP-B (Panel D) particles. All four particle types exhibited 7% to 9% higher JI values than neat plasma analyses, indicating that particle-based fractionation improved assaying consistency. For NP-D, NP-E and NP-A, JI inversely correlated with particle concentration, indicating that lower particle concentration can decrease variation across assay replicates.

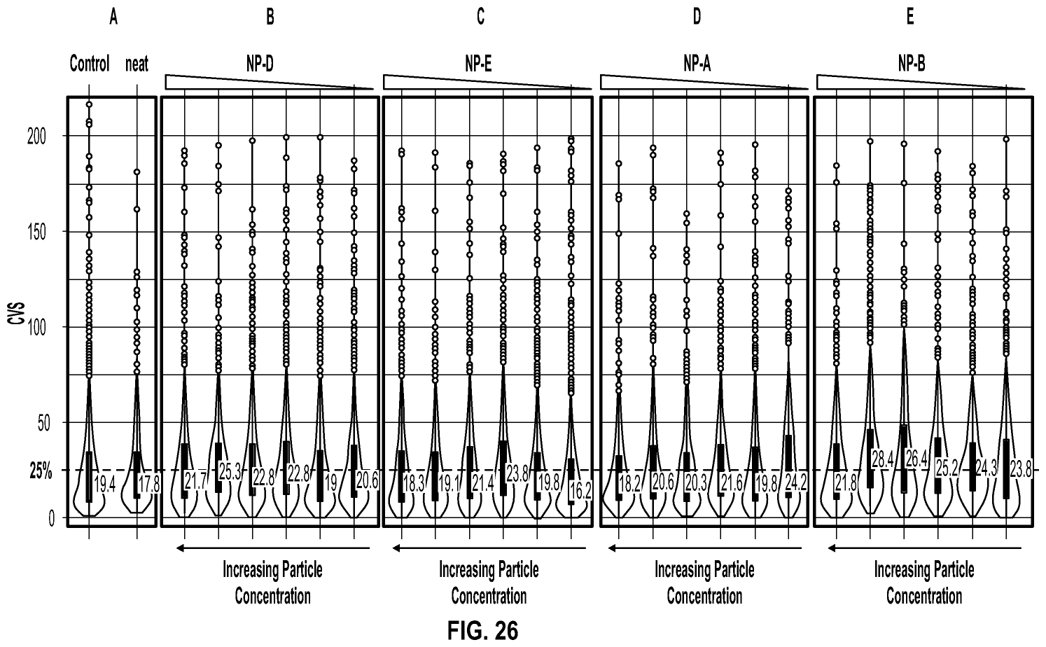

FIG. 26 provides coefficient of variation (CV) values for the protein groups identified in neat plasma (panel A), NP-D (Panel B), NP-E (Panel C), NP-A (Panel D), and NP-B (Panel E). While the CV values for the four particle types may not be significantly lower than the neat plasma, a greater number of low abundance protein groups were detected with the four particles (as compared to neat plasma).

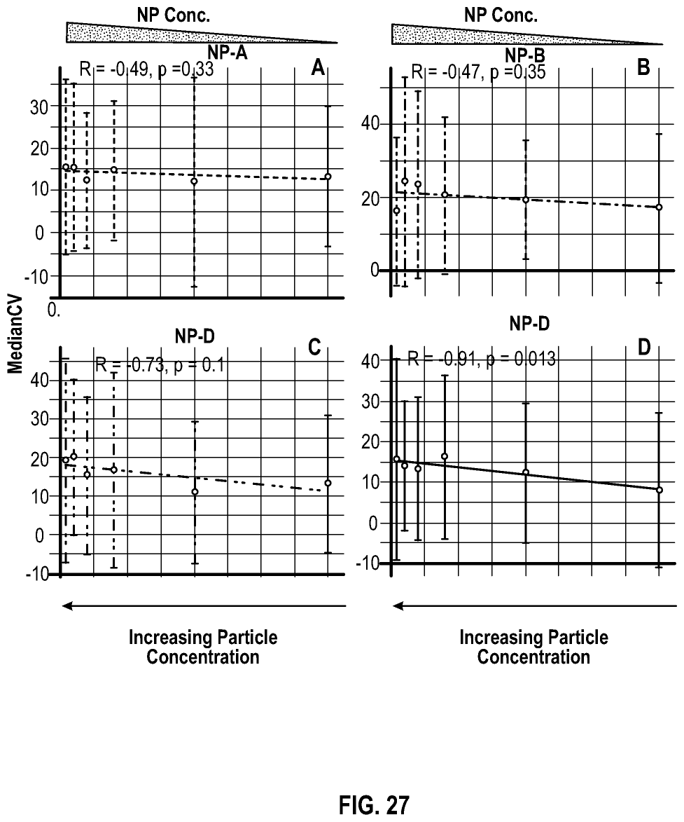

FIG. 27 provides coefficient of variation (CV) values for protein groups commonly identified on NP-D, NP-E, NP-A, and NP-B particles over the range of particle concentrations. The coefficients of variation for the commonly identified proteins correlated with particle concentration, indicating that lower particle concentration can increase assay precision by diminishing variation across replicates.

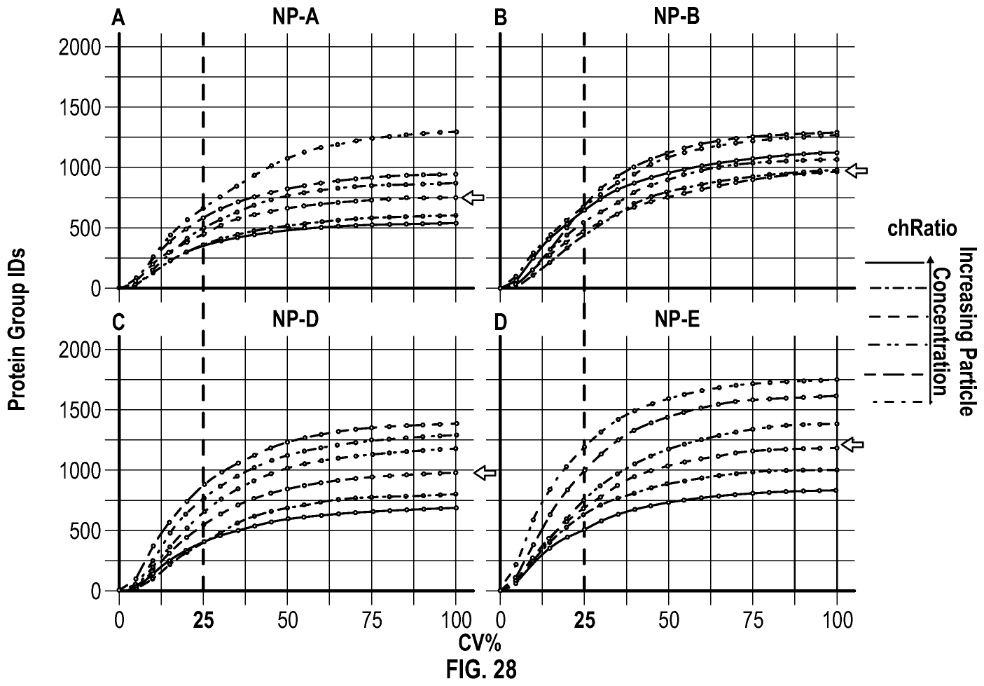

FIG. 28 provides CV accumulation curves for NP-A (Panel A), and NP-B (Panel B), NP-D (Panel C) and NP-E (Panel D) particles at each measured concentration, with each curve corresponding to a different particle concentration. NP-A, NP-D, and NP-E exhibit clean trends for increasing accumulation profiles with decreasing particle concentrations.

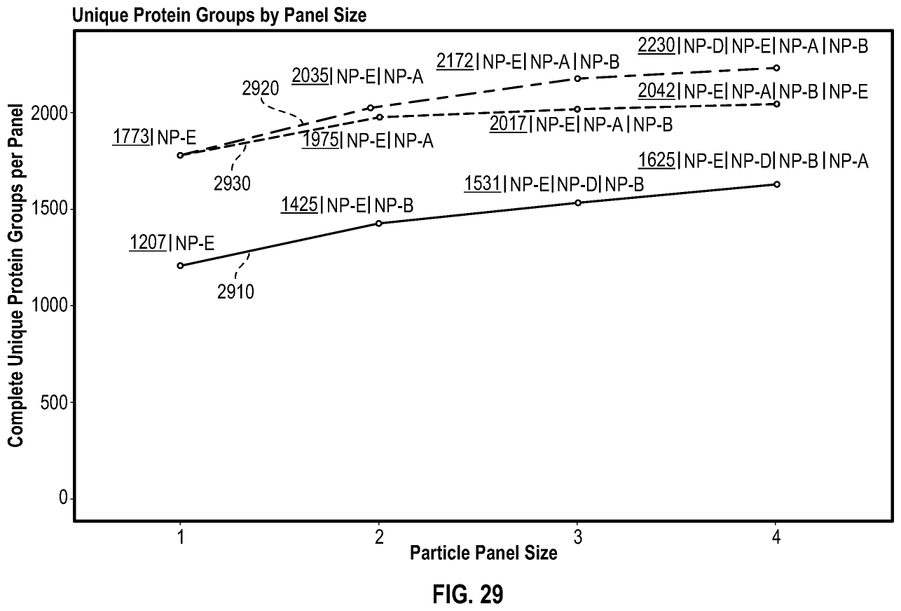

FIG. 29 provides protein group identification numbers for a variety of particle panels as a function of particle panel size (ranging from 1 to 4 particles). Trace 2910 provides optimal particle panel sizes using intermediate particle concentrations. Trace 2920 provides the panels with the highest protein group identification numbers. Trace 2930 provides the panels with the highest protein group identification numbers and peptide yields of at least 1.5 μg. The particle types in each panel are summarized in TABLE 8 below.

TABLE 8
FIG. 29 OPTIMIZED PARTICLE PANELS

| PANEL | | | |
|---|---|---|---|
| SIZE | TRACE 2910 | TRACE 2920 | TRACE 2930 |
| 1 Particle | NP-E | NP-E | NP-E |
| 2 Particles | NP-E, NP-B | NP-E, NP-A | NP-E, NP-A |
| 3 Particles | NP-D, NP-E, | NP-E, NP-A | NP-E, NP-A |
| | NP-B | NP-B | NP-B |
| 4 Particles | NP-D, NP-E, | NP-D, NP-E, | NP-D, NP-E, |
| | NP-A, NP-B | NP-A, NP-B | NP-A, NP-B |

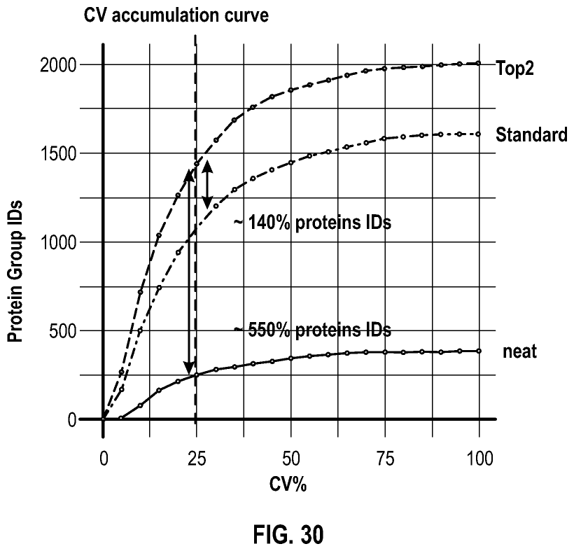

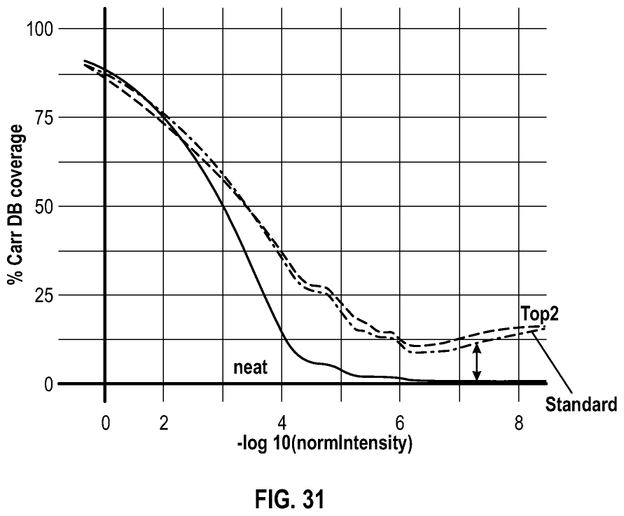

FIG. 30 provides CV accumulation curves for protein group identifications with a low concentration of a two particle panel (NP-E and NP-A), a moderate concentration of a four particle panel (NP-D, NP-E, NP-A and NP-B), and direct analysis of neat plasma. FIG. 31 provides percent coverage of Carr database (Keshishian et al., Mol. Cell Proteomics 14, 2375-2393 (2015)) proteins as a function of protein abundance for the low concentration of the two particle panel (NP-E and NP-A), the moderate concentration of the four particle panel (NP-D, NP-E, NP-A and NP-B), and the neat plasma analysis of FIG. 30. While the particle panels and neat plasma analysis provided similar coverage of high abundance proteins, the particle panels provided higher coverage of moderate and low abundance proteins.

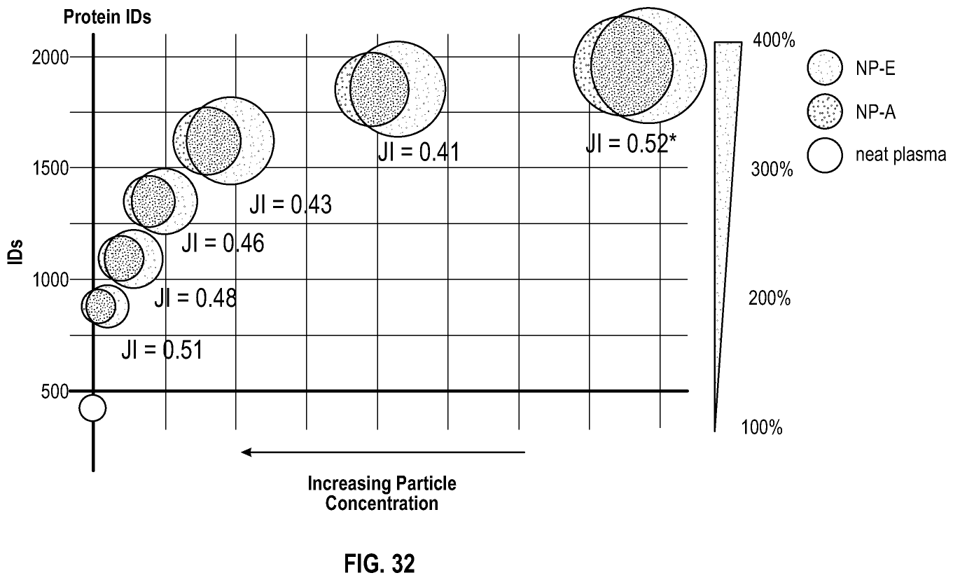

FIG. 32 illustrates protein group identification numbers obtained with varying concentrations of NP-E and NP-A particles. The total number of protein group identifications increased with decreasing particle concentration, with the lowest particle concentration yielding the largest number of protein group identifications and the highest JI between the two particle types.

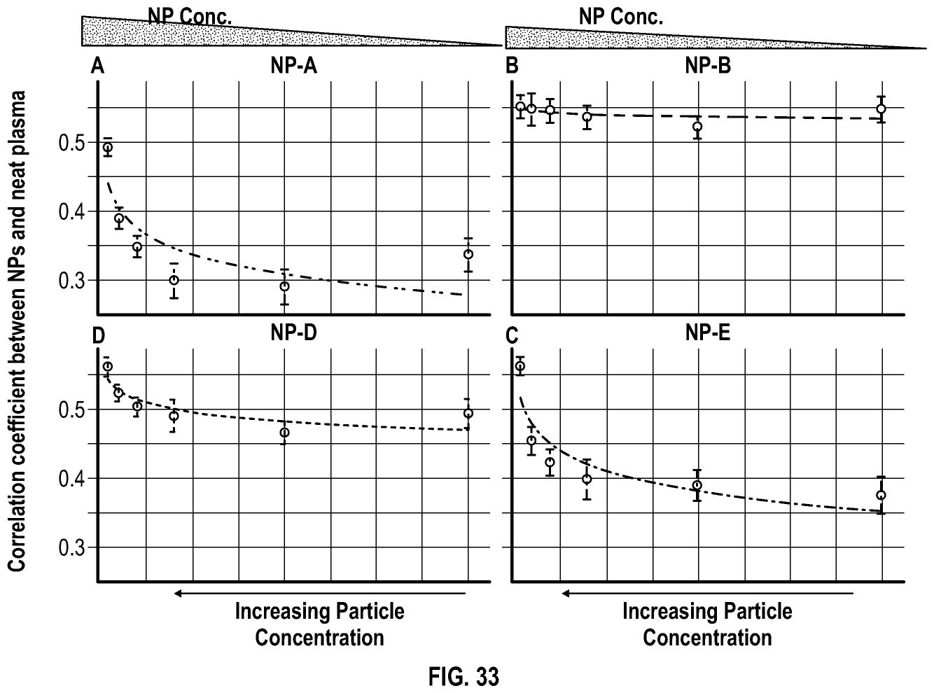

FIG. 33 provides correlation coefficients between the sets of protein groups identified in neat plasma and the sets of protein groups identified on NP-A (panel A), NP-B (panel B), NP-D (panel C) and NP-E (panel D) particles. As can be seen from the plot, decreasing particle concentration decreased correspondence between the sets of protein groups identified with neat plasma and each of the four particles.

### Example 13 Multi-Concentration Particle Assay with Two Particle Panels

This example covers the relationship between particle concentration and protein corona composition for multiple particle types and particle panels. Two separate particle panels spanning eight particle types (summarized in TABLE 9) were utilized in biomolecule corona assays as outlined in Example 12. Briefly, the eight particle types were each separately contacted to diluted human plasma over a large concentration range. Following 1 hour, 37° C. incubations for protein corona formation, supernatant was separated from the particles, and the protein coronas were digested, desalted, and analyzed with LC-MS/MS in data-independent acquisition mode.

TABLE 9
PARTICLE PANELS FOR PROTEIN CORONA ANALYSIS

| Particle | | |
|---|---|---|
| Name | Description | Particle Panel |
| NP-C | Silica-Coated SPION | NP-Panel A, NP-Panel B |
| NP-D | N-(3-Trimethoxysilyl- | NP-Panel A, NP-Panel B |
| | propyl)diethylenetriamine- | |
| | coated SPION | |
| NP-E | Poly(dimethylaminopropyl- | NP-Panel A, NP-Panel B |
| | methacrylamide)-coatedSPION | |
| NP-A | Carboxyl functionalized | NP-Panel A |
| | polystyrene-coated SPION | |
| NP-B | Dextran-coated SPION | NP-Panel A |
| NP-J | 1,6-hexanediamine functionalized | NP-Panel B |
| | SPION | |
| NP-K | Mixed amide, carboxylate | — |
| | functionalized, silica-coated SPION | |
| NP-L | N¹-(3-(trimethoxysilyl)propyl)hexane- | NP-Panel B |
| | 1,6-diamine functionalized, | |
| | silica-coated SPION | |

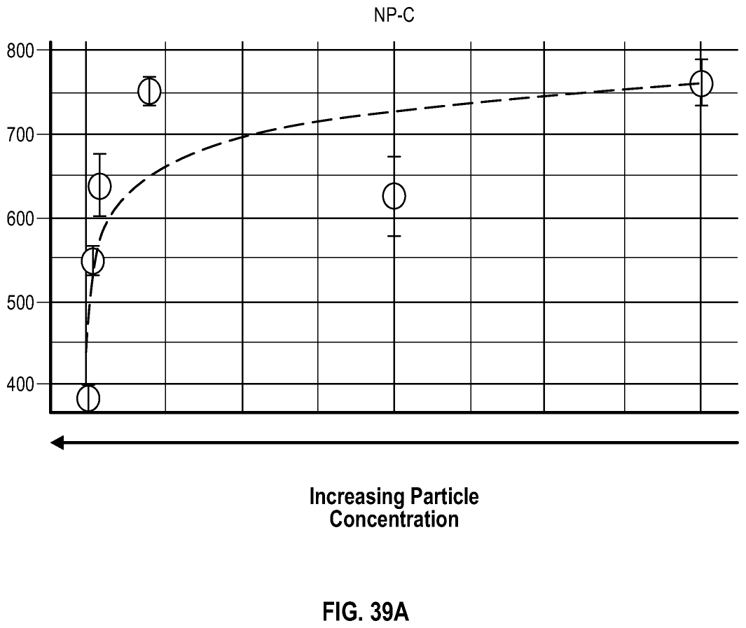

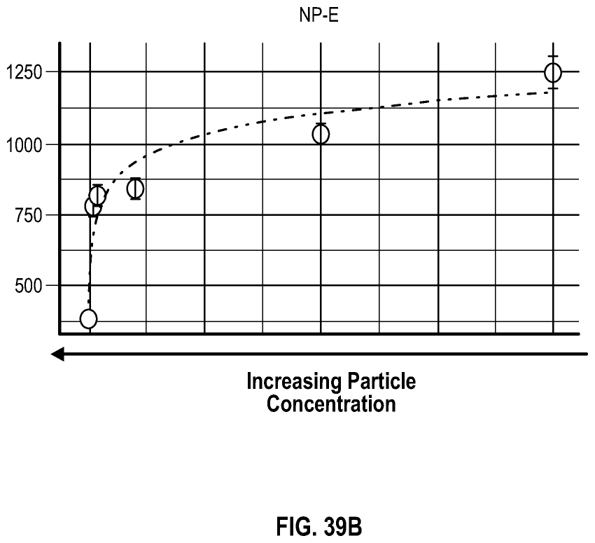

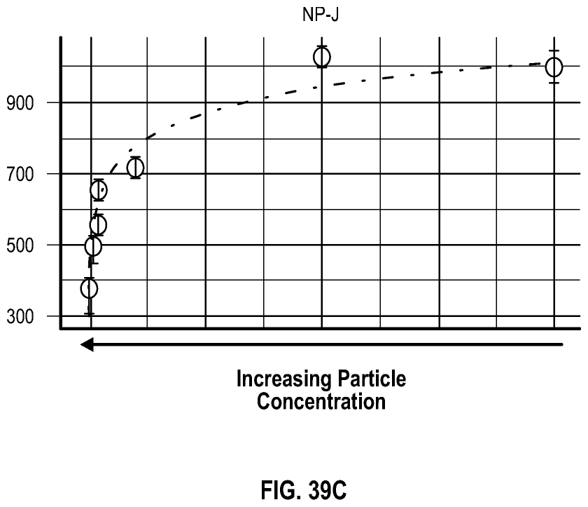

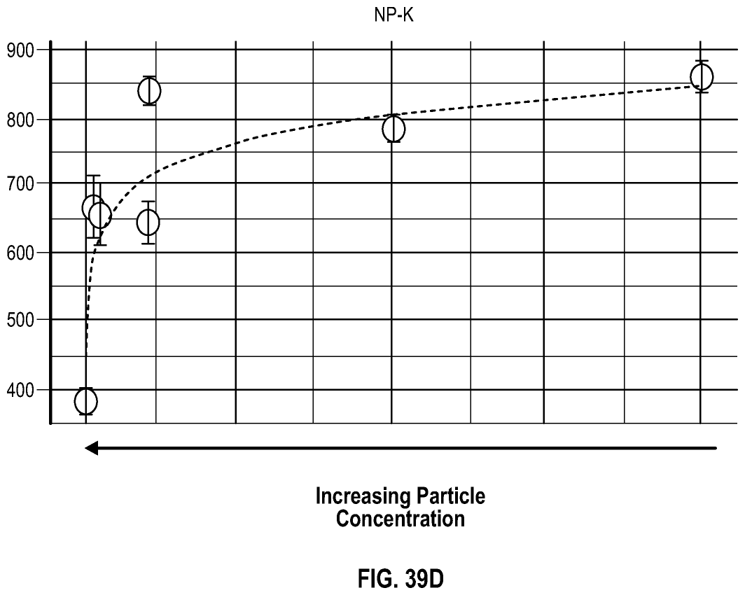

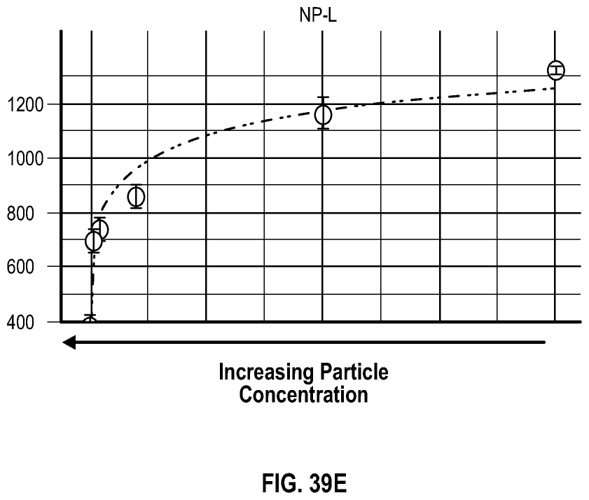

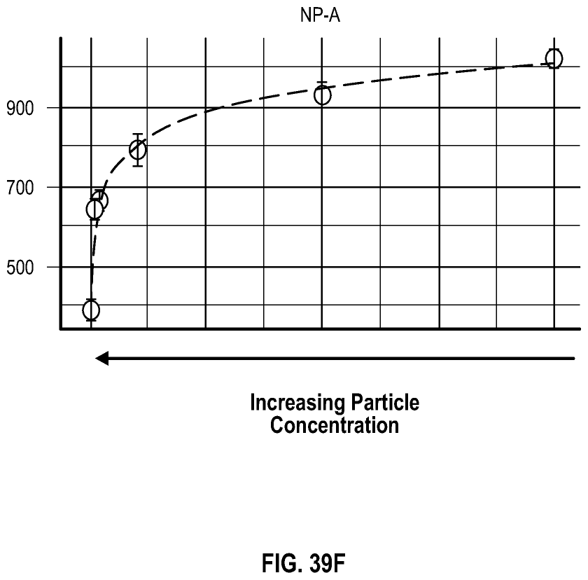

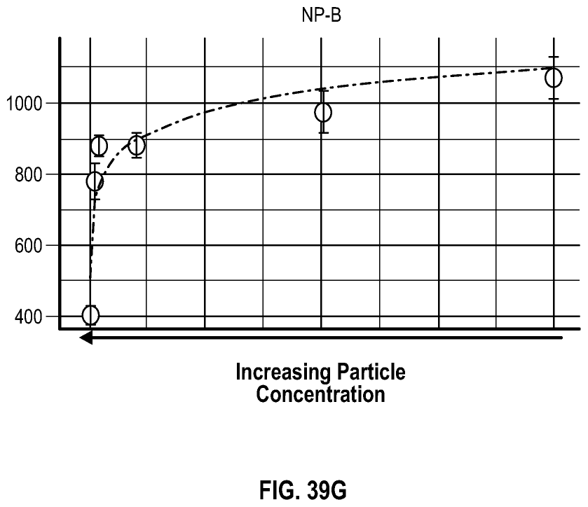

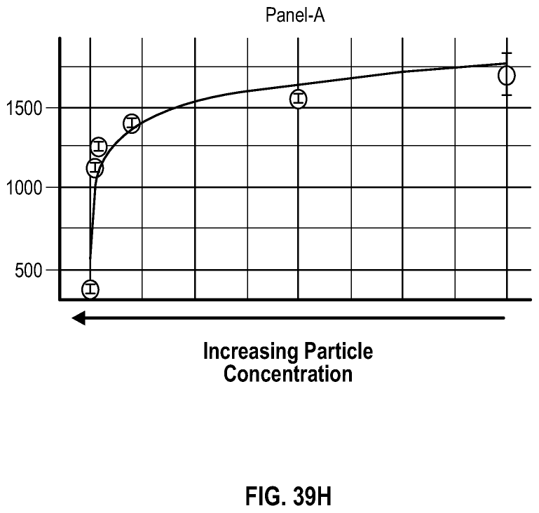

FIG. 39A-I provide the results of protein corona analysis with seven particle types and two particle panels. In each plot, the bottom left data point corresponds to protein group counts from analysis of neat plasma, while the remaining 5 data points provide protein group counts obtained with the particle type or particle panel combined with the plasma over a large particle concentration range. The data confirm that particle dilution can fundamentally alter, and in many cases increase the number of protein groups identified with biomolecule corona analysis. For each particle and particle panel tested, the greatest protein group counts were obtained with the lowest or second lowest particle concentration. Five particle types, namely NP-E (FIG. 39B), NP-J (FIG. 39C), NP-L (FIG. 39E), NP-A (FIG. 39F), and NP-B (FIG. 39G), exhibited increasing protein group counts across the particle dilution series. Two particle types, NP-C(FIG. 39A) and NP-K (FIG. 39D), exhibited higher protein group counts at high particle concentrations than at intermediate particle concentrations, potentially indicating large changes in protein corona composition over the large particle concentration range. The highest protein group counts were obtained with the particle panels NP-Panel A (FIG. 39H) and NP-Panel B (FIG. 39I). For both particle panels, protein group counts were about 50% for the highest particle concentration than for the lowest particle concentration. Furthermore, both particle panels generated clean trends across the measured large particle concentration range, indicating that combining multiple particle types for analysis enhance the effects imparted by serial particle dilution.

### Example 14 Prediction of Accurate Abundance Information from Affinity and Competition-Based Sampling

Relative concentrations of biomolecules captured on surfaces can be different from the true relative concentrations of biomolecules in an original sample. Various factors may contribute to this effect. Without being bound to a particular theory, some factors may be: kinetics and thermodynamics of competitive adsorption of the biomolecules on the surfaces, the concentrations of analytes (including the biomolecules), and biomolecule-biomolecule interactions. This example demonstrates a method for using biomolecule concentrations measured from biomolecules coronas to determine an estimate of the true concentrations of biomolecules in the original sample.

### Ratio Stepping NP-Surface: Analyte Ratio

Without being bound to particular theory, the abundance of a protein in a biomolecule corona may depend on thermodynamic and kinetic effects associated with competitive adsorption. This may be modeled as a function of an unknown affinity and competition coefficient constant Kc. If binding sites are unlimited, it could be expected that the competition between different biomolecules is minimal, and thus, the concentration of proteins in the biomolecule corona can be close to the true in-sample concentration. If binding sites are limited, Kc of each biomolecule can determine the abundance in the corona.

FIGS. 40A-B show PCA projections for neat plasma, and two nanoparticles at various concentrations of nanoparticles. The measurements from different concentrations of NP are grouped together. As the ratio of plasma:nanoparticle increases, the points move further away from the cluster of points of the neat plasma. The points of the two nanoparticles move away in different directions. This illustrates that with more extreme plasma: nanoparticle ratios, the ratio of compression can increase, resulting in more biased data, because the measured abundances are driven more strongly by competitive adsorption. Moreover, NP-specific properties may become more prominent which can result in different NPs having more distinct biomolecule coronas.

FIG. 40C shows the same data in FIGS. 40A-B projected using UMAP.

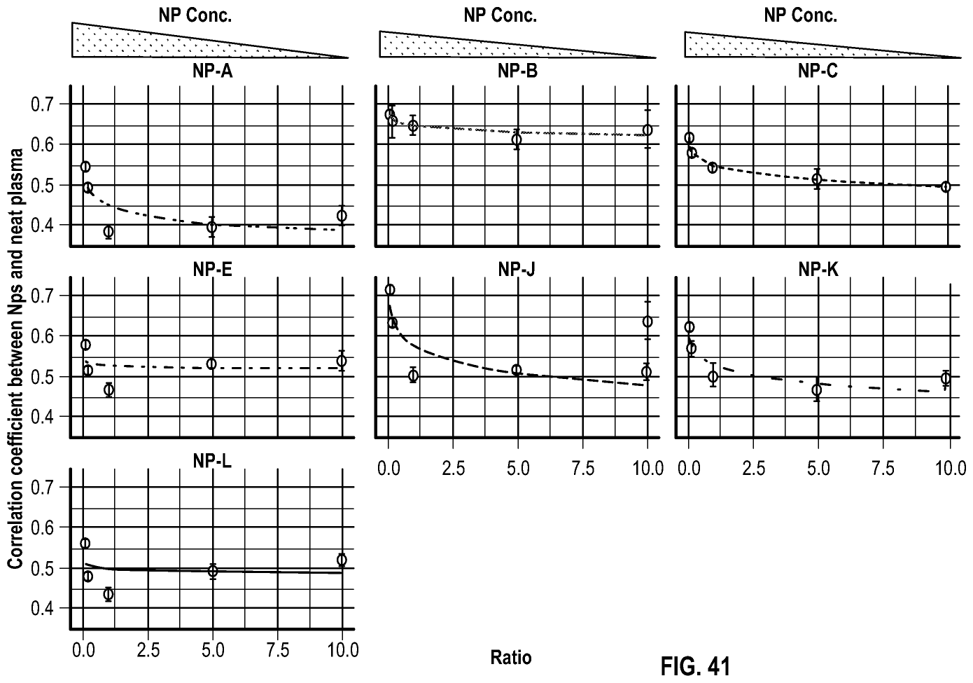

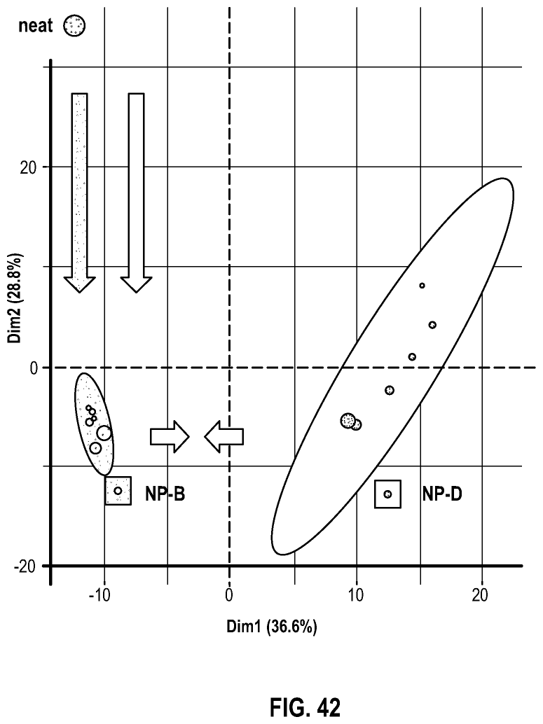

FIG. 41 shows the mean correlation coefficient between biomolecules concentrations measured using the nanoparticles and biomolecule concentrations measured from neat plasma, as a function of plasma: nanoparticle ratios. As the plasma: nanoparticle ratios become more extreme, the mean correlation coefficient decreases for each of the 7 particle types tested. Analyte-specific correction factors can be generated to adjust the abundance measured from protein coronas towards the original in-sample abundance.

### Example 15 Peptide Quantitation and Reconstitution

This example demonstrates a method for performing mass spectrometry on peptides, such that the peptides are delivered in controlled amounts and controlled concentrations to a mass spectrometer.

The amount of peptides in a sample can be determined by contacting reference samples having known amounts of peptide at various dilutions with a fluorescent reagent. The fluorescent reagent can be configured to output a signal that is correlated in strength with the concentration of peptide in the reference samples. Measurements of the fluorescent signal strength can allow one to construct a reference curve (e.g., “peptide quantitation standard curve”) that can be used to determine the amount of peptide in samples having an unknown amount of peptide. The following paragraphs, in part, explain the procedure followed to perform the above steps in detail using an automated system. However, this procedure may also be performed manually.

The automated system comprises a Hamilton STARlet robot configured with 8 independent pipetting channels, a 96-well pipette head (aka Multi-Probe Head), a Hamilton Heater Shaker, Hamilton AutoLoad, labware carriers specific to the SP100 configuration, a Cognex camera, and a Cognex camera bracket specific to the SP100, in addition to other SP100 components. The automated system is programmed using Hamilton Venus.

The automated system is programmed to follow the procedure in Table 10 to determine the peptide standard quantitation curve and the amount of peptide in biological samples.

TABLE 10
EXAMPLE PROCEDURE FOR PEPTIDE QUANTITATION

| Step # | Instructions |
|---|---|
| 1 | Instruct operators to load the SP100 deck with: (i) Hamilton pipette tips (300 ul |
| | conductive nested tips); (ii) 2 mL deep well plate containing user samples; (iii) One |
| | 60 mL trough/reservoir containing 12 mL of Assay Buffer; (iv) Greiner 96-well |
| | black microplate; (v) One 2mL tube containing 100 μL of TFS Peptide Digest Assay |
| | Standard; (vi) Eight empty 2 mL cryovials for peptide standard quantitation curve |
| | preparation; (vii) Eight 2 mL cryovials containing 310 μL of Assay reagent per tube; |
| | (viii) One 4-well plate containing Elution Solution in one well; and (ix) One standard |
| | 96-well plate; (x) One standard 96-well plate with black (opaque) lid. |
| 2 | Load the SP100 carriers using Hamilton Autoload while verifying barcodes of the |
| | pipette tips, 2 mL deep well plate, 4-well plate, and 2 mL cryovials. |
| 3 | Aliquot 100 μL of Assay Buffer to each well of the empty standard 96-well plate. |
| 4 | Fill the first empty standard curve tube with 564 μL of Elution Solution and the |
| | remaining seven tubes with 100 μL of Elution Solution. |
| 5 | Transfer 36 μL of assay standard to the first peptide standard quantitation curve tube |
| | containing 564 μL of Elution Solution then mix. |
| 6 | Perform serial dilution into peptide standard quantitation curve tubes 2-7 by |
| | transferring 150 μL and mixing each time. |
| 7 | Transfer 70 μL of Assay Buffer from the standard 96-well plate to the black |
| | microplate using the multi-probe head. |
| 8 | Transfer 10 μL of each sample from the deep well plate to the black microplate. |
| 9 | Transfer 10 μL of each standard from the standard dilution tubes to column 12 of the |
| | black microplate. |
| 10 | Transfer 20 μL of Assay Reagent to every well of the black microplate. |
| 11 | Start a 5 minute timer. |
| 12 | Move the black microplate to the Hamilton Heater Shaker for 20 seconds at 700 |
| | RPM |
| 13 | Move the black microplate back to its original location. |
| 14 | Unload the carriers using the Hamilton Autoload. |
| 15 | Prompt the user that their plate can be read in a fluorimeter when the 5 minute timer |
| | is complete. |

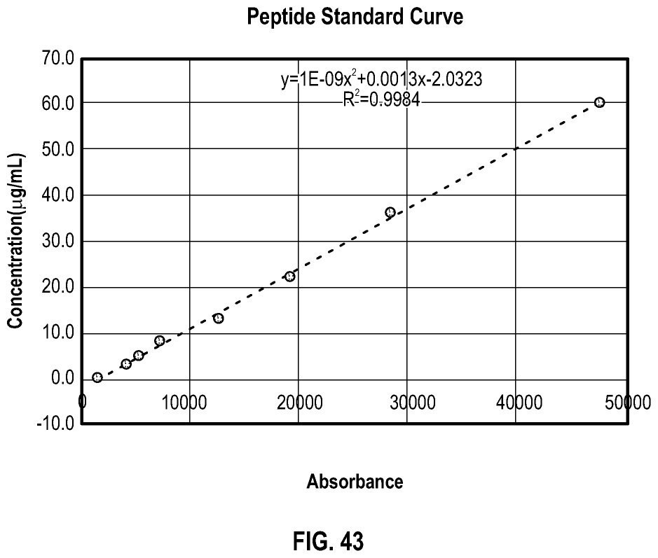

The method uses the TFS standard reagent instead to create a peptide standard quantitation curve of 60 μg/ml to 2.8 μg/ml with a 0 μg/ml blank). The method establishes the peptide standard quantitation curve by creating a serial dilution series of seven concentrations plus an eighth zero-mass (empty) well. FIG. 43 shows an example of a peptide standard quantitation curve. The peptide standard quantitation curve concentrations are 60.0, 36.0, 21.6, 13.0, 7.8, 4.7, and 2.8 μg/ml. The peptide standard quantitation curve is used to determine the amount of peptide in the biological samples provided. Given a known volume of a sample, and the fluorescence measured, the mass of peptide can be calculated. Hamilton Venus specific liquid class definitions were created for TFS Assay Buffer, TFS Elution Solution, TFS Assay Reagent, and TFS Peptide Digest Assay Standard, specific to volumes used in the script.

After the amount of peptide in the assay plates are determined, the method continues by drying samples in the assay plates, and then reconstituting the samples. Reconstituting can be useful in cases where samples have a concentration that is lower than an optimal concentration for injection into a liquid chromatography column and/or mass spectrometer, or in cases where samples have a volume that is far higher than an optimal concentration for injection into a liquid chromatography column and/or mass spectrometer. Reconstitution allows the samples to be injected into a mass spectrometer at a predetermined concentration and/or volume.

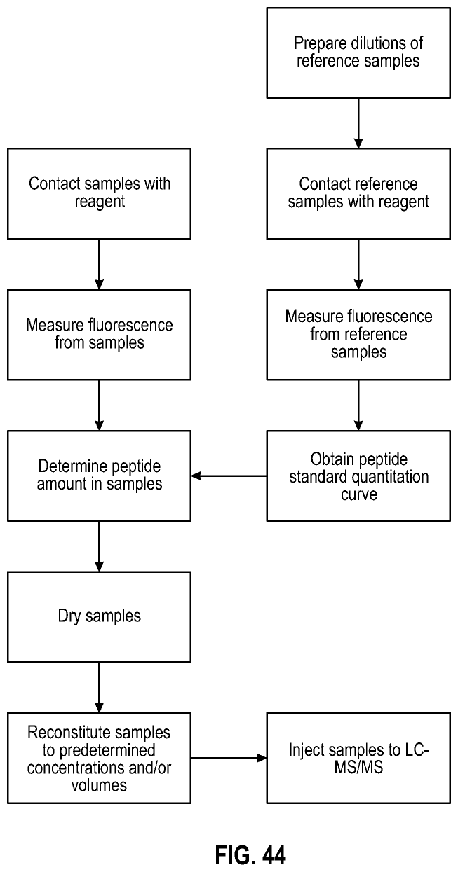

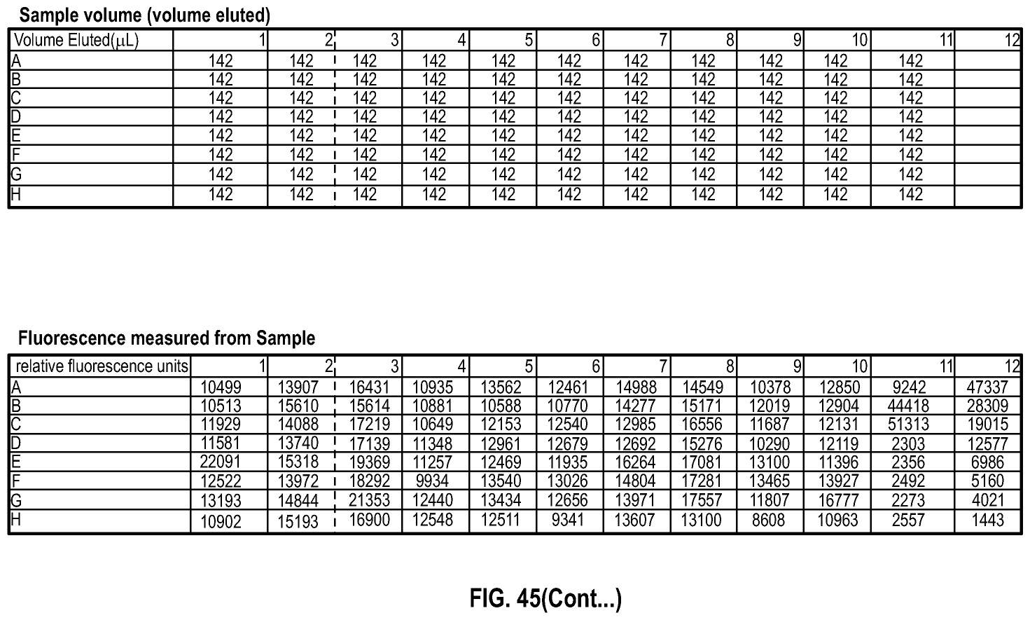

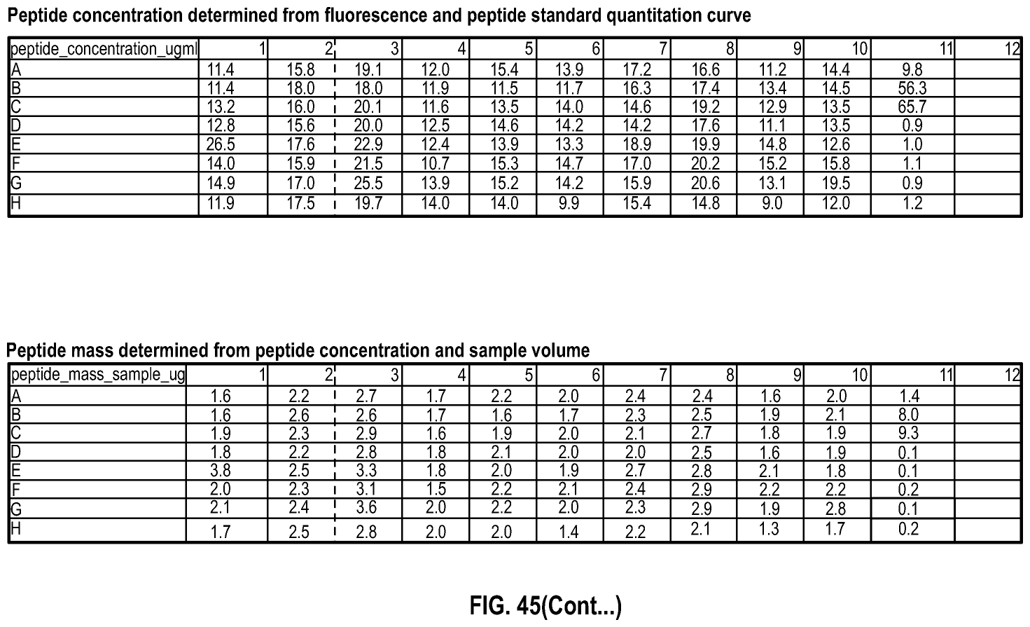

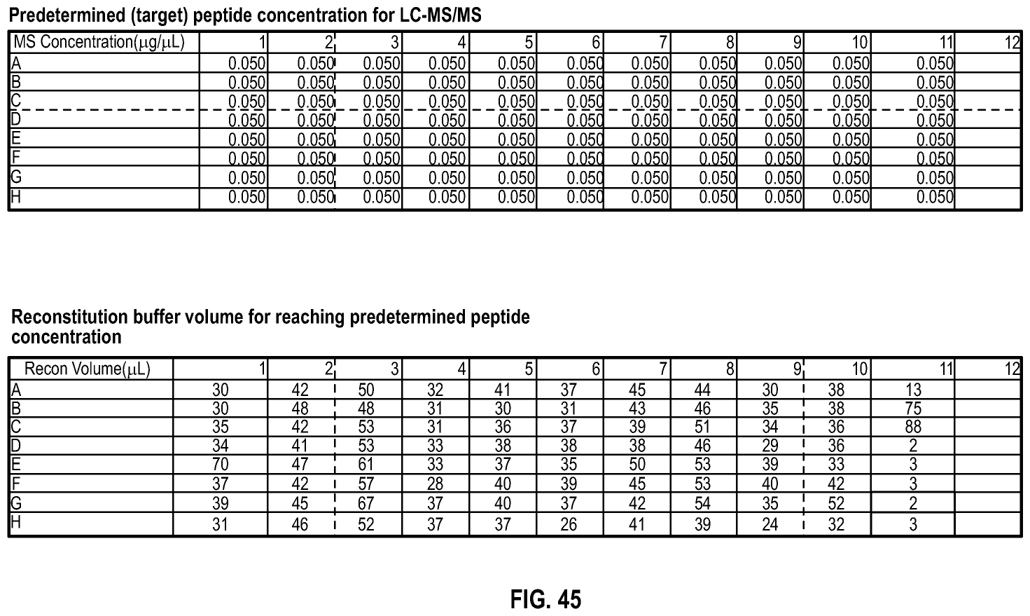

Samples are dried on-plate under vacuum overnight. Then, reconstitution buffer is added to the samples to prepare them for injection at predetermined concentrations and/or volumes. The predetermined concentrations and/or volumes may be configured independently based on each well. For example, the predetermined concentrations and/or volumes may be configured based on the particle type used to prepare the peptides. The predetermined concentrations and/or volumes may be configured for control samples. FIG. 44 shows a process diagram for peptide quantitation and reconstitution.

### Numbered Embodiments

The following list of numbered embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for determining a concentration or an amount of a biomolecule or biomolecule group in a biological sample, the method comprising: (a) contacting the biological sample with a plurality of particle-containing solutions each having a different particle concentration, to generate a plurality of biomolecule coronas each corresponding to an individual solution of the plurality of particle-containing solutions; (b) assaying the plurality of biomolecule coronas for a dataset comprising data corresponding to one or more biomolecules or biomolecule groups comprising the biomolecule or biomolecule group in the biological sample; and (c) determining the concentration or the amount of the biomolecule or the biomolecule group in the biological sample based at least partially on the dataset, wherein the determining is made in the absence of using a reference biomolecule external to the biological sample.

Embodiment 2. The method of embodiment 1, wherein at least a subset of the plurality of particle-containing solutions differ in particle concentration by at least 1, 2, 3, 4, 5, 6, 7, 8, 9, or 10 orders of magnitude. Embodiment 3. The method of any one of embodiments 1-2, wherein at least a subset of the plurality of particle-containing solutions differ in particle concentration by at most 1, 2, 3, 4, 5, 6, 7, 8, 9, or 10 orders of magnitude.

Embodiment 4. The method of any one of embodiments 1-3, wherein solutions of the plurality of particle-containing solutions have particle concentrations between 1 μg/ml and 100 mg/ml. Embodiment 5. The method of any one of embodiments 1-3, wherein solutions of the plurality of particle-containing solutions have particle concentrations of at least 1, 2, 3, 4, 5, 6, 7, 8, 9, 10, 20, 30, 40, 50, 60, 70, 80, 90, 100, 200, 300, 400, 500, 600, 700, 800, or 900 μg/ml. Embodiment 6. The method of any one of embodiments 1-3, wherein solutions of the plurality of particle-containing solutions have particle concentrations of at most 1, 2, 3, 4, 5, 6, 7, 8, 9, 10, 20, 30, 40, 50, 60, 70, 80, 90, 100, 200, 300, 400, 500, 600, 700, 800, or 900 μg/ml. Embodiment 7. The method of any one of embodiments 1-3, wherein solutions of the plurality of particle-containing solutions have particle concentrations of at least 1, 2, 3, 4, 5, 6, 7, 8, 9, 10, 20, 30, 40, 50, 60, 70, 80, 90, or 100 mg/ml. Embodiment 8. The method of any one of embodiments 1-3, wherein solutions of the plurality of particle-containing solutions have particle concentrations of at most 1, 2, 3, 4, 5, 6, 7, 8, 9, 10, 20, 30, 40, 50, 60, 70, 80, 90, or 100 mg/ml.

Embodiment 9. The method of any one of embodiments 1-8, wherein the plurality of biomolecule coronas are associated with a single particle type in solutions of the plurality of particle-containing solutions. Embodiment 10. The method of any one of embodiments 1-9, wherein each of the particle-containing solutions comprises a same particle type. Embodiment 11. The method of any one of embodiments 1-10, wherein each of the particle-containing solutions comprises a different particle type. Embodiment 12. The method of any one of embodiments 1-11, wherein each of the particle-containing solutions comprises a same particle panel comprising a plurality of different particles.

Embodiment 13. The method of any one of embodiments 1-12, wherein particles in an individual solution of the plurality of particle-containing solutions have a polydispersity of less than 1 or 0.5. Embodiment 14. The method of any one of embodiments 1-13, wherein particles in an individual solution of the plurality of particle-containing solutions have a polydispersity of greater than 1 or 0.5. Embodiment 15. The method of any one of embodiments 13-14, wherein the polydispersity is determined at least in part by size variance of the particles. Embodiment 16. The method of any one of embodiments 13-14, wherein the polydispersity is determined at least in part by mass variance of the particles.

Embodiment 17. The method of any one of embodiments 1-16, wherein a solution of the plurality of particle-containing solutions comprises a surface modified particle. Embodiment 18. The method of any one of embodiments 1-17, wherein a solution of the plurality of particle-containing solutions comprises a plurality of surface modified particles. Embodiment 19. The method of embodiment 18, wherein the plurality of surface modified particles comprises particles having different physicochemical properties. Embodiment 20. The method of embodiment 19, wherein the physicochemical properties comprise size, charge, core material, shell material, porosity, density, hydrophobicity, hydrophilicity, charge, rigidity, or any combination thereof.

Embodiment 21. The method of any one of embodiments 1-20, wherein the dataset comprises a plurality of signals corresponding to the plurality of biomolecule coronas. Embodiment 22. The method of embodiment 21, wherein the plurality of signals comprises optical signals, electrical signals, or a combination thereof. Embodiment 23. The method of any one of embodiments 1-21, wherein the dataset comprises a plurality of datasets. Embodiment 24. The method of any one of embodiments 21-23, wherein the determining of (c) comprises comparing intensities of the plurality of signals against an intensity of a reference signal. Embodiment 25. The method of embodiment 24, wherein the reference signal is associated with a biomolecule intrinsic to the sample. Embodiment 26. The method of embodiment 25, wherein the biomolecule intrinsic to the sample comprises albumin, globulin, transferrin, fibrinogen, antitrypsin, al-acid glycoprotein, apolipoprotein, ceruloplasmin, transthyretin, a complement factor, or any combination thereof.

Embodiment 27. The method of any one of embodiments 1-26, wherein the dataset comprises training data for a machine learning algorithm.

Embodiment 28. The method of any one of embodiments 1-27, wherein the contacting of (a) for each of the plurality of particle-containing solutions is for about a same duration of time. Embodiment 29. The method of embodiment 28, wherein the same duration of time is shorter than the equilibration times of the plurality of particle-containing solutions subsequent to the contacting of (a).

Embodiment 30. The method of any one of embodiments 1-29, wherein the one or more biomolecules or biomolecule groups comprise a plurality of biomolecules or biomolecule groups, and wherein the determining of (c) comprises identifying a concentration or an amount of each of the plurality of biomolecules or biomolecule groups in the biological sample. Embodiment 31. The method of embodiment 30, wherein concentrations of the plurality of biomolecules or biomolecule groups are identified in a single assay.

Embodiment 32. The method of any one of embodiments 1-31, wherein the biomolecule or biomolecule group comprises a protein or protein group.

Embodiment 33. The method of any one of embodiments 1-32, wherein the concentration of the biomolecule or the biomolecule group is less than about 10 μg/ml, 1 μg/ml, 100 ng/ml, 10 ng/ml, 1 ng/ml, or 100 μg/ml. Embodiment 34. The method of any one of embodiments 1-33, wherein the concentration of the biomolecule or the biomolecule group is greater than about 10 μg/ml, 1 μg/ml, 100 ng/ml, 10 ng/ml, 1 ng/ml, or 100 μg/ml. Embodiment 35. The method of any one of embodiments 1-34, wherein the biomolecule or biomolecule group comprises a plurality of human plasma proteins or human plasma protein groups, and wherein the plurality of human plasma proteins or human plasma protein groups comprises at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 proteins or protein groups. Embodiment 36. The method of any one of embodiments 1-35, wherein the biomolecule or biomolecule group comprises a plurality of human plasma proteins or human plasma protein groups, and wherein the plurality of human plasma proteins or human plasma protein groups comprises at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 proteins or protein groups. Embodiment 37. The method of any one of embodiments 21-36, wherein the determining of (c) comprises determining concentrations of the at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 proteins or protein groups based at least partially on intensities of the plurality of signals. Embodiment 38. The method of any one of embodiments 21-37, wherein the determining of (c) comprises determining concentrations of the at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 proteins or protein groups based at least partially on intensities of the plurality of signals. Embodiment 39. The method of any one of embodiments 1-38, wherein the assaying of (b) comprises separating the plurality of biomolecule coronas from the biological sample. Embodiment 40. The method of embodiment 39, wherein the separating comprises magnetically separating the plurality of biomolecule coronas from the biological sample. Embodiment 41. The method of any one of embodiments 1-40, wherein the assaying of (b) comprises digesting the one or more biomolecules or biomolecule groups. Embodiment 42. The method of any one of embodiments 1-41, wherein the determining of (c) comprises identifying relative abundances of a plurality of isoforms of a protein.

Embodiment 43. The method of any one of embodiments 1-42, wherein a particle concentration of the plurality of the particle-containing solutions is approximately equal to a total protein concentration of the biological sample. Embodiment 44. The method of any one of embodiments 1-43, wherein the contacting the biological sample with the plurality of particle-containing solutions comprises combining at most about 250 μL of the biological sample with at most about 250 μL of a particle-containing solution of the plurality of particle-containing solutions. Embodiment 45. The method of any one of embodiments 1-44, wherein the contacting the biological sample with the plurality of particle-containing solutions comprises combining at most about 100 μL of the biological sample with at most about 100 μL of a particle-containing solution of the plurality of particle-containing solutions. Embodiment 46. The method of any one of embodiments 1-43, wherein the contacting the biological sample with the plurality of particle-containing solutions comprises adding at least about 100 nL of plasma per cm² of particle surface area to each solution of the plurality of particle-containing solutions. Embodiment 47. The method of any one of embodiments 1-44, wherein the contacting the biological sample with the plurality of particle-containing solutions comprises adding between about 100 nL and 100 mL of plasma per cm² of particle surface area to each solution of the plurality of particle-containing solutions.

Embodiment 48. The method of any one of embodiments 1-47, wherein the biological sample is diluted by at least 2, 3, 4, 5, 6, 7, 8, 9, or 10-fold prior to the contacting with the plurality of particle-containing solutions. Embodiment 49. The method of any one of embodiments 1-48, wherein the biological sample is diluted by at most 2, 3, 4, 5, 6, 7, 8, 9, or 10-fold prior to the contacting with the plurality of particle-containing solutions.

Embodiment 50. The method of any one of embodiments 1-49, wherein particles of the plurality of particle-containing solutions have diameters between about 100 and about 500 nanometers. Embodiment 51. The method of any one of embodiments 1-50, wherein particles of the plurality of particle-containing solutions have diameters between about 100 and about 300 nanometers. Embodiment 52. The method of any one of embodiments 1-51, wherein particles of the plurality of particle-containing solutions comprise diameters of at least about 500 nanometers. Embodiment 53. The method of any one of embodiments 1-52, wherein particles of the plurality of particle-containing solutions comprise diameters of at most about 200 nanometers.

Embodiment 54. The method of any one of embodiments 1-53, wherein the plurality of particle-containing solutions comprises a particle selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. Embodiment 55. The method of any one of embodiments 1-54, wherein the plurality of particle-containing solutions comprises at least two particles selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. Embodiment 56. The method of any one of embodiments 1-55, wherein the plurality of particle-containing solutions comprises at least three particles selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. Embodiment 57. The method of any one of embodiments 1-56, wherein the plurality of particle-containing solutions comprises at least four particles selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. Embodiment 58. The method of any one of embodiments 1-57, wherein the plurality of particle-containing solutions comprises a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. Embodiment 59. The method of any one of embodiments 1-58, wherein the plurality of particle-containing solutions comprises a superparamagnetic iron oxide particle (SPION) comprising a poly(N-(3-(dimethylamino) propyl) methacrylamide) (PDMAPMA) surface. Embodiment 60. The method of any one of embodiments 1-59, wherein the plurality of particle-containing solutions comprises a superparamagnetic iron oxide particle (SPION) comprising a poly(oligo (ethylene glycol) methyl ether methacrylate) (POEGMA) surface. Embodiment 61. The method of any one of embodiments 1-60, wherein the plurality of particle-containing solutions comprises a superparamagnetic iron oxide particle (SPION) comprising an N-(3-Trimethoxysilylpropyl) diethylenetriamine surface. Embodiment 62. The method of any one of embodiments 1-61, wherein the plurality of particle-containing solutions comprises a superparamagnetic iron oxide particle (SPION) comprising a Poly(dimethyl aminopropyl methacrylamide) (Dimethylamine) surface. Embodiment 63. The method of any one of embodiments 1-62, wherein the plurality of particle-containing solutions comprises a superparamagnetic iron oxide particle (SPION) comprising a dextran surface. Embodiment 64. The method of any one of embodiments 1-63, wherein the plurality of particle-containing solutions comprises a superparamagnetic iron oxide particle (SPION) comprising a surface with a mixed chemistry based on amine-epoxy chemistry. Embodiment 65. The method of any one of embodiments 1-64, wherein the plurality of particle-containing solutions comprises a superparamagnetic iron oxide particle (SPION) comprising a Polyzwitterion coated (Poly(N-[3-(Dimethylamino) propyl]methacrylamide-co-[2-(methacryloyloxy)ethyl]dimethyl-(3-sulfopropyl) ammonium hydroxide, P (DMAPMA-co-SBMA)) surface. Embodiment 66. The method of any one of embodiments 1-65, wherein the plurality of particle-containing solutions comprises a superparamagnetic iron oxide particle (SPION) comprising styrene surface comprising an oleic acid functionalization. Embodiment 67. The method of any one of embodiments 1-66, wherein the plurality of particle-containing solutions comprises a superparamagnetic iron oxide particle (SPION) comprising a boronated styrene surface. Embodiment 68. The method of any one of embodiments 1-67, wherein the plurality of particle-containing solutions comprises a superparamagnetic iron oxide particle (SPION) comprising a carboxylated styrene surface. Embodiment 69. The method of any one of embodiments 1-68, wherein the plurality of particle-containing solutions comprises a superparamagnetic iron oxide microparticle (SPION) comprising a carboxylated styrene surface. Embodiment 70. The method of any one of embodiments 1-69, wherein the plurality of particle-containing solutions comprises a superparamagnetic iron oxide microparticle (SPION) comprising a strongly acidic silica surface.

Embodiment 71. The method of any one of embodiments 1-70, wherein the dataset comprises a plurality of factors or a plurality of functions that account for the differences between one or more amounts of the one or more biomolecules or biomolecule groups in the one or more biomolecule coronas. Embodiment 72. The method of embodiment 71, wherein the plurality of factors or the plurality of functions are specific to the particle. Embodiment 73. The method of embodiment 71 or 72, wherein the plurality of factors or the plurality of functions are specific to the biomolecule or the biomolecule group.

Embodiment 74. The method of any one of embodiments 1-73, wherein the concentration or the amount of the biomolecule or the biomolecule group is correlated with an intrinsic concentration or an intrinsic amount of the biomolecule or the biomolecule group measured from the biological sample without contacting with a particle-containing solution, with a Pearson correlation coefficient of at least 0.1, 0.2, 0.3, 0.4, 0.5, 0.6, 0.7, 0.8, or 0.9. Embodiment 75. The method of any one of embodiments 1-73, wherein the concentration or the amount of the biomolecule or the biomolecule group is correlated with an intrinsic concentration or an intrinsic amount of the biomolecule or the biomolecule group measured from the biological sample without contacting with a particle-containing solution, with a Pearson correlation coefficient of at most 0.1, 0.2, 0.3, 0.4, 0.5, 0.6, 0.7, 0.8, 0.9, or 1.0.

Embodiment 76. A method for determining a concentration or an amount of a plurality of protein groups in a biological sample, the method comprising: (a) contacting a reference biological sample with (i) a first particle-containing solution comprising a first concentration of a particle to generate a first protein corona and (ii) a second particle-containing solution comprising a second concentration of the particle to generate a second protein corona, wherein the first concentration is higher than the second concentration; (b) performing mass spectrometry using (i) the first protein corona to determine a first plurality of protein group intensities of the plurality of protein groups in the first protein corona and (ii) the second protein corona to determine a second plurality of protein group intensities of the plurality of protein groups in the second protein corona; (c) determining a plurality of factors or a plurality of functions that account for the differences between the first plurality of protein group intensities and the second plurality of protein group intensities, wherein each of the plurality of factors or the plurality of functions are specific to the particle and to each protein group in the plurality of protein groups; (d) contacting the biological sample with a third particle-containing solution comprising a third concentration of the particle to generate a third protein corona, wherein the third concentration is less than or equal to about the first concentration, and greater than or equal to about the second concentration; (e) assaying the third protein corona to determine a third plurality of protein group intensities of the plurality of protein groups in the third protein corona; and (f) applying the plurality of factors or the plurality of functions to the plurality of protein group intensities to determine a fourth plurality of protein group intensities, such that the Pearson correlation coefficient is at least 0.5 between the fourth plurality of protein group intensities and intensities of the protein groups measured by performing mass spectrometry on the biological sample without contacting with a particle-containing solution.

Embodiment 77. A method for performing mass spectrometry, comprising: (a) providing a biological sample comprising one or more peptides and a solvent, wherein the one or more peptides comprise proteolytically cleaved derivatives of proteins adsorbed on a surface; (b) determining an amount of the one or more peptides in the biological sample; (c) drying the biological sample to remove at least a portion of the solvent; (d) reconstituting the biological sample with a second solvent, based at least in part on the amount of the one or more peptides, such that the biological sample comprises a predetermined concentration of the one or more peptides; and (e) assaying the biological sample.

Embodiment 78. The method of embodiment 77, wherein the one or more peptides comprise a plurality of peptides, and the assaying comprises determining a relative amount between at least two peptides in the plurality of peptides

Embodiment 79. The method of embodiment 77 or 78, wherein the surface comprises a sensor element surface. Embodiment 80. The method of embodiment 79, wherein the sensor element surface comprises a particle surface. Embodiment 81. The method of embodiment 80, wherein the particle surface is a nanoparticle surface. Embodiment 82. The method of embodiment 80, wherein the particle surface is a microparticle surface. Embodiment 83. The method of any one of embodiments 80-82, wherein the particle surface comprises pores. Embodiment 84. The method of any one of embodiments 80-83, wherein the proteins are bound on the surface via adsorption. Embodiment 85. The method of any one of embodiments 80-84, wherein the proteins are bound on the surface via non-specific binding. Embodiment 86. The method of any one of embodiments 80-84, wherein the proteins are bound on the surface via specific binding. Embodiment 87. The method of any one of embodiments 80-86, wherein the proteins form a corona on the particle surface.

Embodiment 88. The method of any one of embodiments 77-87, wherein the predetermined concentration is based at least in part on one or more physicochemical properties of the surface. Embodiment 89. The method of embodiment 88, wherein the one or more physicochemical parameters comprise: sample to surface ratio, incubation time, pH, salt concentration, ionic strength, solvent composition, solvent dielectric constant, crowding agent concentration, temperature, sample composition, surfactant concentration, concentration of enzymes, activity of enzymes, chemical reactions, concentrations of small molecules, surface chemistry, or any combination thereof.

Embodiment 90. The method of any one of embodiments 77-89, wherein the determining comprises contacting the biological sample with a reagent configured to output a signal, wherein a strength of the signal is correlated with the amount of the one or more peptides in the biological sample. Embodiment 91. The method of embodiment 90, wherein the reagent comprises a fluorescing reagent and the signal comprises a fluorescent signal.

Embodiment 92. The method of any one of embodiments 77-91, further comprising, prior to (a), proteolytically cleaving the proteins to generate the one or more peptides. Embodiment 93. The method of any one of embodiments 77-92, further comprising, prior to proteolytically cleaving, contacting the proteins with the surface.

Embodiment 94. The method of any one of embodiments 77-93, wherein the drying comprises drying using vacuum.

Embodiment 95. The method of any one of embodiments 77-94, wherein the assaying comprises mass spectrometry. Embodiment 96. The method of embodiment 95, wherein the mass spectrometry comprises liquid-chromatography tandem mass spectrometry (LC-MS/MS).

Embodiment 97. The method of any one of embodiments 77-94, wherein the assaying comprises protein sequencing. Embodiment 98. The method of any one of embodiments 77-94, wherein the assaying comprises binding each protein in the proteins to a pair of antibodies. Embodiment 99. The method of embodiment 98, wherein the pair of antibodies comprises complementary single-stranded nucleic acid sequences attached thereto, such that when the pair of antibodies bind to the molecule, the complementary nucleic acids hybridize to form a double stranded nucleic acid. Embodiment 100. The method of embodiment 99, wherein the double stranded nucleic acid is configured to form a binding complex with a polymerase and a plurality of nucleotides, nucleosides, nucleotide analogs, and/or nucleoside analogs to perform an amplification reaction to produce a detectable signal. Embodiment 101. The method of any one of embodiments 77-94, wherein the assaying comprises binding a protein in the proteins to an aptamer. Embodiment 102. The method of any one of embodiments 77-94, wherein the assaying comprises an immunoassay. Embodiment 103. The method of any one of embodiments 78-102, further comprising performing (a)-(e) for a second plurality of peptides in serial or in parallel.

Embodiment 104. The method of any one of embodiments 77-103, wherein the biological sample is derived from a complex biological sample. Embodiment 105. The method of embodiment 104, wherein the biological sample is derived from plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. Embodiment 106. The method of embodiment 105, wherein the biological sample is derived from plasma or serum.

Embodiment 107. A method for performing mass spectrometry, comprising: (a) providing a substrate comprising a plurality of wells or chambers, wherein the plurality of wells or chambers comprises: (i) a first well or chamber comprising a first biological sample therein, wherein the first biological sample comprises a first set of peptides and a first solvent, wherein the first set of peptides comprises proteolytically cleaved derivatives of a first set of proteins adsorbed on a first surface; and (ii) a second well or chamber comprising a second biological sample therein, wherein the second biological sample comprises a second set of peptides and a second solvent, wherein the second set of peptides comprises proteolytically cleaved derivatives of a second set of proteins adsorbed on a second surface; (b) determining (i) a first amount of the first set of peptides in the first biological sample and (ii) a second amount of the second set of peptides in the second biological sample; (c) drying (i) the first biological sample to remove at least a portion of the first solvent and (ii) the second biological sample to remove at least a portion of the second solvent; (d) reconstituting (i) the first biological sample with a first buffer based at least in part on the first amount and (ii) the second biological sample with a second buffer based at least in part on the second amount, such that the first biological sample and the second biological sample comprises about a predetermined concentration of peptides; (e) injecting (i) the first biological sample into a mass spectrometer to generate a first set of peptide intensities and (ii) the second biological sample into the mass spectrometer to generate a second set of peptide intensities; and (f) generating a dataset comprising the first set of peptide intensities and the second set of peptide intensities, wherein a bias arising from differences in input concentration of peptides into the mass spectrometer is normalized between the first set of peptide intensities and the second set of peptide intensities, such that the first set of peptide intensities and the second set of peptide intensities are proportional to a common reference without further renormalization.

Embodiment 108. A method for performing mass spectrometry, comprising: (a) providing a first biological sample comprising a first set of peptides and a first solvent, wherein the first set of peptides comprises proteolytically cleaved derivatives of a first set of proteins adsorbed on a first surface; (b) determining a first amount of the first set of peptides in the first biological sample; (c) drying the first biological sample to remove at least a portion of the first solvent; (d) reconstituting the first biological sample with a first buffer based at least in part on the first amount, such that the first biological sample comprises about a predetermined concentration of peptides; (e) injecting the first biological sample into a mass spectrometer to generate a first set of peptide intensities; (f) providing a second biological sample comprising a second set of peptides and a second solvent, wherein the second set of peptides comprises proteolytically cleaved derivatives of a second set of proteins adsorbed on a second surface; (g) determining a second amount of the second set of peptides in the second biological sample; (h) drying the second biological sample to remove at least a portion of the second solvent; (i) reconstituting the second biological sample with a second buffer based at least in part on the second amount, such that the second biological sample comprises about the predetermined concentration of peptides; (j) injecting the second biological sample into a mass spectrometer to generate a second set of peptide intensities; and (k) generating a dataset comprising the first set of peptide intensities and the second set of peptide intensities, wherein a bias arising from differences in input concentration of peptides into the mass spectrometer is normalized between the first set of peptide intensities and the second set of peptide intensities, such that the first set of peptide intensities and the second set of peptide intensities are proportional to a common reference without further renormalization.

Embodiment 109. A computer program product comprising a computer-readable medium having computer-executable code encoded therein, the computer-executable code adapted to be executed to implement any one of the methods of embodiments 1-108.

Embodiment 110. A non-transitory computer-readable storage media encoded with a computer program including instructions executable by one or more processors to implement any one of the methods of embodiments 1-108.

Embodiment 111. A computer-implemented system comprising: a digital processing device comprising: at least one processor, an operating system configured to perform executable instructions, a memory, and a computer program including instructions executable by the digital processing device to perform any one of the methods of embodiments 1-108.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-18. (canceled)

19. A method for performing mass spectrometry, comprising:
(a) providing a biological sample comprising one or more peptides and a solvent, wherein the one or more peptides comprise proteolytically cleaved derivatives of proteins on a surface;
(b) determining an amount of the one or more peptides in the biological sample;
(c) drying the biological sample to remove at least a portion of the solvent;
(d) reconstituting the biological sample with a second solvent, based at least in part on the amount of the one or more peptides, such that the biological sample comprises a predetermined concentration of the one or more peptides; and
(e) assaying the biological sample.

20.-21. (canceled)

22. The method of claim 19, wherein the one or more peptides comprise a plurality of peptides, and the assaying comprises determining a relative amount between at least two peptides in the plurality of peptides.

23. The method of claim 19, wherein the surface comprises a particle surface.

24. The method of claim 23, wherein the particle surface is a nanoparticle surface or a microparticle surface.

25. The method of claim 23, wherein the particle surface comprises pores.

26. The method of claim 19, wherein the proteins are bound on the surface via adsorption.

27. The method of claim 23, wherein the proteins form a corona on the particle surface.

28. The method of claim 19, wherein the proteins are bound on the surface via non-specific binding.

29. The method of claim 19, wherein the proteins are bound on the surface via specific binding.

30. The method of claim 19, wherein the predetermined concentration is based at least in part on one or more physicochemical properties of the surface.

31. The method of claim 30, wherein the one or more physicochemical properties comprise: sample to surface ratio, incubation time, pH, salt concentration, ionic strength, solvent composition, solvent dielectric constant, crowding agent concentration, temperature, sample composition, surfactant concentration, concentration of enzymes, activity of enzymes, chemical reactions, concentrations of small molecules, surface chemistry, or any combination thereof.

32. The method of claim 19, wherein the determining comprises contacting the biological sample with a reagent configured to output a signal, wherein a strength of the signal is correlated with the amount of the one or more peptides in the biological sample.

33. The method of claim 32, wherein the reagent comprises a fluorescing reagent and the signal comprises a fluorescent signal.

34. The method of claim 19, further comprising, prior to (a), proteolytically cleaving the proteins to generate the one or more peptides.

35. The method of claim 34, further comprising, prior to proteolytically cleaving, contacting the proteins with the surface.

36. The method of claim 19, wherein the assaying is selected from the group consisting of mass spectrometry, protein sequencing, binding each protein in the proteins to a pair of antibodies, binding a protein in the proteins to an aptamer, and an immunoassay.

37. The method of claim 36, wherein the mass spectrometry comprises liquid-chromatography tandem mass spectrometry (LC-MS/MS).

38. The method of claim 36, wherein the pair of antibodies comprises complementary single-stranded nucleic acid sequences attached thereto, such that when the pair of antibodies bind to the molecule, the complementary nucleic acids hybridize to form a double stranded nucleic acid.

39. The method of claim 38, wherein the double stranded nucleic acid is configured to form a binding complex with a polymerase and a plurality of nucleotides, nucleosides, nucleotide analogs, and/or nucleoside analogs to perform an amplification reaction to produce a detectable signal.

40. The method of claim 19, further comprising performing (a)-(e) for a second plurality of peptides in serial or in parallel.
